(19) United States
(12) Patent Application Publication
(10) Pub. No.: US 2026/0027215 A1
(43) Pub. Date: Jan. 29, 2026

(54) COMPOSITIONS FOR TRANSPORT OF THERAPEUTIC CARGOS USING BINDERS TARGETING CA-IV

(71) Applicants: Receptive Bio, Inc., Thousand Oaks, CA (US); California Institute of Technology, Pasadena, CA (US)

(72) Inventors: Viviana Gradinaru, Pasadena, CA (US); Xiaozhe Ding, Thousand Oaks, CA (US); Tsui-Fen Chou, 91125, CA (US); Yanping Qiu, Pasadena, CA (US)

(21) Appl. No.: 19/280,935

(22) Filed: Jul. 25, 2025

Related U.S. Application Data
(60) Provisional application No. 63/675,999, filed on Jul. 26, 2024.

(51) Int. Cl. A61K 47/55

(52) U.S. Cl. CPC A61K 47/55

(57) ABSTRACT

The present invention provides small molecules that act as binders for the BBB-crossing through the receptor carbonic anhydrase IV (CA-IV).

### FIELD OF THE INVENTION

The invention relates to methods and shuttles for crossing the blood brain barrier.

### BACKGROUND

The blood brain barrier (BBB) presents a fundamental bottleneck to the development of effective research tools and therapeutics for the central nervous system (CNS). This structure, comprising mainly of brain endothelial cells, requires large molecules to be delivered via invasive intracranial injections, technically challenging focused ultrasound, or receptor-mediated transcytosis. The rational design of BBB-crossing large molecules has long been hampered by the imperfect understanding of the mechanisms involved in transcytosis, with only a handful of targets, such as the transferrin receptor, validated for research and therapies.

Thus, the identification of BBB-crossing targets, mechanisms, molecules and methods is needed to improve the efficiencies of research tools and therapies for CNS.

### SUMMARY

The present invention provides conjugates and their methods of use comprising molecules that act as binders for the BBB-crossing through the receptor carbonic anhydrase IV (CA-IV). Advantageously, the present invention benefits from the surprising discovery that known bioavailable small molecules, including previously approved FDA drugs, may act as CA-IV binders. Unexpectedly, these CA-IV binding small molecules were not known to act as BBB transcytosis shuttles when conjugated to a therapeutic payload prior to the instant invention.

Accordingly, aspects of the invention provide the present invention conjugates that comprise a carbonic anhydrase IV (CA-IV) binder and a therapeutic cargo conjugated to the CA-IV binder. The invention relies on the surprising discovery that these small molecule binders of CA-IV may be utilized in conjugates for delivering therapeutic cargo across the BBB.

In aspects of the invention, the binder or CA-IV binder is a small molecule drug that was previously approved by the FDA. For example, the small molecule may be selected from the group consisting of foretinib, tanespimycin, nilotinib, tozasertib, ivacaftor, linsitinib, epirubicin HCl, zosuquidar 3HCl, ziprasidone HCl, acetylcysteine, zafirlukast, elvitegravir, meclizine 2HCl, candesartan cilexetil, diclazuril, flunarizine 2HCl, evacetrapib, estradiol valerate, etravirine, montelukast sodium, nelfinavir mesylate, mefloquine HCl, pimozide, zolpidem, and hydroxyprogesterone caproate, or a derivative thereof. Unexpectedly, before the present invention, none of the above small molecules were known to bind to CA-IV, and were not developed to be utilized in conjugates for delivering therapeutic payload across BBB.

The therapeutic cargo may be conjugated to the binder via a linker. The therapeutic cargo may be covalently conjugated to the binder, for example via bioconjugation.

The therapeutic cargo may be a biological molecule. For example, the biological molecule is selected from the group consisting of a nucleic acid (for example, RNA, siRNA, DNA, or an ASO), a protein (for example, an enzyme), a peptide, an antibody, a nanobody, a lipid, a polysaccharide, and a combination thereof.

The therapeutic cargo may be a non-biological molecule, for example a small molecule. The therapeutic cargo may be a protein binder, for example chimeric small molecule therapeutic. For example, the therapeutic cargo may be a proteolysis targeting chimera (PROTAC).

Advantageously, conjugates of the invention are characterized by delivery of the therapeutic cargo across the blood brain barrier (BBB). The therapeutic cargo may be a therapeutic cargo for the treatment of a disorder affecting the central nervous system.

In certain aspects of the invention, the CA-IV binder binds to the human CA-IV protein. Accordingly, the conjugates provided herein are utilized to deliver the therapeutic cargo across BBB in humans.

Accordingly, when provided to a cell expressing CA-IV as a surface protein, binding of the small molecule binder to the CA-IV protein mediates transcytosis of the therapeutic cargo across the BBB.

Aspects of the invention further provide methods of delivering therapeutic cargo across the BBB of a subject. Methods of the invention comprise providing to a subject a conjugate comprising a carbonic anhydrase IV (CA-IV) binder and a therapeutic cargo conjugated to the binder. The binder may be discovered by a screening method comprising the steps of adding to a buffer CA-IV and a plurality of small molecules and measuring binding of the plurality of small molecules to CA-IV. For example, the adding step may comprise adding CA-IV and each of the small molecules from the plurality of small molecules into separate compartments, for example separate wells in a multiwell plate or separate droplets in a microfluidic assay. CA-IV may be added to the buffer as cells and the cells separated into separate compartments together with a small molecule from the plurality of small molecules. As a consequence, each compartment acts as a reaction vessel for CA-IV and a single small molecule.

Advantageously, the plurality of small molecules may be small molecule drugs that have not shown binding to CA-IV. For example, the binder discovered may be selected from the group consisting of foretinib, tanespimycin, nilotinib, tozasertib, ivacaftor, linsitinib, epirubicin HCl, zosuquidar 3HCl, ziprasidone HCl, acetylcysteine, zafirlukast, elvitegravir, meclizine 2HCl, candesartan cilexetil, diclazuril, flunarizine 2HCl, evacetrapib, estradiol valerate, etravirine, montelukast sodium, nelfinavir mesylate, mefloquine HCl, pimozide, zolpidem, and hydroxyprogesterone caproate, or a derivative thereof.

### BRIEF DESCRIPTION OF THE DRAWINGS

FIG. 1 is a workflow of a screening and validation process with FDA-approved drugs for identifying CA-IV binders in a library of the FDA-approved drugs.

FIG. 2-18 are graphs of plate-by-plate screening results from the screening with FDA-approved drugs.

FIG. 19-24 are graphs of representative dose-response curves for select hits from the initial screen for FDA-approved drugs.

### DETAILED DESCRIPTION

The present invention provides small molecules that act as binders for the BBB-crossing through the receptor carbonic anhydrase IV (CA-IV). The invention further provides conjugates comprising said binders for delivering therapeutic cargo across BBB.

### Receptors for Enhanced Blood-Brain Barrier Crossing

Blood-brain barrier (BBB) has emerged as a complex, dynamic, adaptable interface that controls the exchange of substances between the central nervous system (CNS) and the blood, to prevent the uncontrolled leakage of substances from the blood into the brain. The cells that make up the structure of the BBB include mostly brain endothelial cells, which constantly communicate with the other cells of the CNS (e.g., astrocytes, microglia, neurons, mast cells and pericytes, as well as circulating immune cells), adapting their behaviors to serve the needs of the CNS, responding to pathological conditions, and in some cases participating in the onset, maintenance or progression of disease. The complexity of BBB functions explains much of the difficulty in developing drugs that can cross the BBB. Utilizing receptors on the BBB interface can offer a method of crossing BBB.

The present invention provides binders for receptors on the BBB interface and methods of using the same to enhance BBB crossing and CNS potency, such as increasing the permeability of the BBB and delivering a therapeutic agent across the BBB to a nervous system, specifically carbonic anhydrase IV. Without being bound by any theory, the novel target receptors disclosed herein may facilitate enhanced BBB receptor-mediated transcytosis across various species, including mammals such as human.

In some embodiments, a method of increasing permeability of the BBB comprises providing a binder capable of binding to a BBB crossing receptor (e.g., carbonic anhydrase IV), thereby increasing permeability of the BBB (e.g., through transcytosis). In some embodiments, at least one activity of the BBB-crossing receptor (e.g., carbonic anhydrase IV) can be reduced through binding to a small molecule. Accordingly, in some embodiments, a method of increasing permeability of the BBB comprises reducing the activity of carbonic anhydrase IV, thereby increasing permeability of the BBB. In some embodiments, a binder binds to one or more of the zinc binding site (e.g., a catalytic pocket) and substrate binding site of the carbonic anhydrase IV. The carbonic anhydrase IV can be a vertebrate carbonic anhydrase IV including non-human primates and humans. In some embodiments, the carbonic anhydrase IV is a mouse carbonic anhydrase IV (Car4), a human carbonic anhydrase IV (CA4), or a variant or a homolog thereof.

### Carbonic Anhydrase IV

The present invention provides binderss for the BBB-crossing suing the receptor carbonic anhydrase IV, capable of facilitating the delivery of a pharmaceutical agent across the BBB (CA-IV binders). Carbonic anhydrase IV is an isozyme that belongs to the carbonic anhydrase family, a family of zinc metalloenzymes, which catalyzes the reversible reaction of hydration of CO₂ (H₂O+CO₂−HCO₃—+H+), allowing the enzyme to regulate intra- and extra-cellular concentrations of CO₂, H+, and HCO₃—. The carbonic anhydrases participate in a variety of biological processes, including respiration, calcification, acid-base balance, bone resorption, and the formation of aqueous humor, cerebrospinal fluid, saliva, and gastric acid. The carbonic anhydrases show extensive diversity in tissue distribution and in their subcellular localization. There are at least seven genetically distinct isozymes of mammalian carbonic anhydrase, designated I-VII, each of which catalyzes the reversible hydration of carbon dioxide by a zinc-hydroxide mechanism. Physiological functions that are regulated by carbonic anhydrase comprise, for example, removal of HCO₃— in lung by respiration, reutilization of HCO₃— in kidney, production of aqueous humor in eyes, cerebrospinal fluids in brain, gastric juice production in stomach, pancreatic juice, and bone resorption by osteoclasts. Carbonic anhydrase family members also play important roles in metabolic processes that include ureagenesis, gluconeogenesis, and lipogenesis.

Different from other carbonic anhydrases that are either soluble or attached to the plasma membrane by a membrane-spanning domain, carbonic anhydrase IV is a glycosylphosphatidyl-inositol-anchored membrane isozyme. Carbonic anhydrase IV is broadly conserved across vertebrates and has similar CNS expression profiles in humans, with a recent single cell analysis of human brain vasculature confirming CA4's expression in the human BBB. Carbonic anhydrase IV has been shown to regulate pH, which is associated with neural discharge and can influence neuronal function through ion-gated channels.

In some embodiments, the carbonic anhydrase IV disclosed herein is a human carbonic anhydrase IV (CA4). CA4 is known to localize on the luminal surface of brain endothelial cells throughout the cortex and cerebellum where it enzymatically modulates carbon dioxide-bicarbonate balance. Human CA4 has been previously characterized as a 35-kDa protein with a “high activity” in CO2 hydration and a higher activity than other isozymes in catalyzing the dehydration of HCO3-. In general, human CA4 contains an 18-amino acid signal sequence at the N-terminal of the protein for endoplasmic reticulum (ER) translocation and a 260-amino acid “CA domain” containing active site amino acid residues that shows 30-36% homology with cytoplasmic CAs. At the C-terminal, an additional 27 amino acid residues containing the hydrophobic sequences of 21 amino acids sufficient to span the membrane are preceded by the 6-amino acid signal sequence for GPI-anchoring. The amino acid residue, Ser 266, was identified as the site for the attachment of the GPI anchor. The removal of C-terminal hydrophobic domain found in the CA4 precursor has important impact on GPI-anchoring, cell surface expression, and realization of the enzyme activity. Based on amino acid sequences deduced from the nucleotide sequence, human CA4 contains no classical consensus sites (Asn-Xxx-Ser/Thr) for N-glycosylation. Human CA4 also contains no oligosaccharide chains, while other mammalian carbonic anhydrase IV (e.g. mouse carbonic anhydrases IV (Car4)) are glycoproteins with one to several oligosaccharide side chains.

In some embodiments, the carbonic anhydrase IV disclosed herein is a mouse carbonic anhydrase IV (Car4). Car4 has recently been found to be among the mouse proteins most strongly positively correlated with plasma-protein uptake in the brain (slightly stronger than the often-targeted transferrin receptor). This property is useful for identifying receptors for enhanced BBB crossing. Car4 is also expressed in the GI tract, kidney, and lung, as well as taste receptor cells where it allows the sensing of carbonation. Mouse Car4 and human CA4 are highly homologous, containing the same amino acids at positions crucial for enzyme activity (e.g., histidine residue 64 (His 64)), with several differences including, for example, that mouse Car4 is an N-linked glycoprotein and the CO2 hydration rate catalyzed by mouse Car4 is much lower than human CA4. Without being bound by any theory, the lower enzyme activity of mouse Car4 may be associated with the replacement of Gly 63 in human CA4 with Gln 63, among several other amino acid replacements. Another difference between mouse Car4 and human CA4 is the Val-131-Asp-136 segment (130's segment) that forms an α-helix in mouse Car4 and an extended loop in human CA4.

In some embodiments, a carbonic anhydrase IV (Car4) disclosed herein as a receptor for enhancing BBB crossing can be any carbonic anhydrase IV, such as a mouse Car4, a human CA4, or a homology or a variant thereof. Carbonic anhydrase IV homologs and/or variants can be derived from a vertebrate species including, but not limited to, mouse, rat, human, bovine, rabbit, monkey, pig, horse, rainbow trout, chimpanzee, squirrel, chicken, goat, and sheep. Carbonic anhydrase IV homologs from various species can be found in public databases identifiable to a person skilled in the art, including for example UniProt, NCBI, and Swiss-Prot.

In some embodiments, a small molecule can interact with a carbonic anhydrase IV disclosed herein (e.g., mouse Car4, human CA4 or a homology or a variant thereof), thereby increasing permeability of the BBB (e.g., through transcytosis). In some embodiments, the increase in the permeability of the BBB is achieved by altering (e.g., increasing or decreasing) the carbonic anhydrase IV activity, such as reducing its activity.

In some embodiments, the alteration of carbonic anhydrase IV activity is achieved by a the binder interacting to one or more active sites of the carbonic anhydrase IV including the zinc binding site and the hydrophobic substrate binding pocket. For example, the binder can interact with the zinc binding site, the hydrophobic substrate binding pocket, or both.

The zinc binding site in carbonic anhydrase IV has a conserved structure dominated by a β-sheet super-structure with a metal binding site formed by at least three His residues. Without being bound by any theory, it is believed that the zinc binding site is on one face of the β-sheet at the bottom of a 15-Å-deep, conical active site cleft in which zinc is liganded by three His residues and hydroxide ion with tetrahedral geometry. The hydrophobic substrate binding pocket is adjacent to zinc-bound hydroxide, formed in large part by bulky residues such as Val at its base and Val, Trp and Leu at its neck. This pocket is highly conserved among all active isozymes on the basis of phylogenetic comparisons. Without being bound by any theory, it is believed that the hydrophobic pocket has a minimum width and depth for efficient catalysis, and linear free energy relationships indicate that the volume of the amino acid residue at the base of the pocket and the hydrophobicity of residues at the neck of the pocket are critical for activity. Both the zinc binding site and the hydrophobic substrate binding pocket are highly conserved among carbonic anhydrase isozymes.

### Select Small Molecule Binders

By the present invention, for the first time, it was discovered that small molecules, including drugs approved by the FDA for different indications, also bind to CA-IV. The invention further beneficially recognized that as a result of the binding of these small molecules to CA-IV, these molecules may be utilized as shuttles for BBB-crossing using the receptor carbonic anhydrase IV, capable of facilitating the delivery of a pharmaceutical agent across the BBB.

For example, by the present invention, it was discovered that the following small molecule drugs may act as CA-IV binders: foretinib, tanespimycin, nilotinib, tozasertib, ivacaftor, linsitinib, epirubicin HCl, zosuquidar 3HCl, ziprasidone HCl, acetylcysteine, zafirlukast, elvitegravir, meclizine 2HCl, candesartan cilexetil, diclazuril, flunarizine 2HCl, evacetrapib, estradiol valerate, etravirine, montelukast sodium, nelfinavir mesylate, mefloquine HCl, pimozide, zolpidem, and hydroxyprogesterone caproate. Each of the drugs, by the present invention, was shown to exhibit CA-IV binding activity.

The present invention also provides methods of identifying small molecules, including approved FDA drugs, as CA-IV binders.

Foretinib is an orally bioavailable small molecule—chemical formula C₃₄H₃₄F₂N₄O; 1-N′-[3-fluoro-4-[6-methoxy 7-(3-morpholin-4-ylpropoxy)quinolin-4-yl]oxyphenyl]-1-N-(4-fluorophenyl)cyclopropane-1,1-dicarboxamide -having the structure:

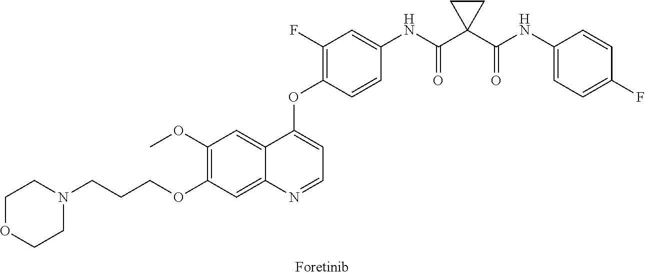 [SMILES: COc1cc2c(Oc3ccc(NC(=O)C4(C(=O)Nc5ccc(F)cc5)CC4)cc3F)ccnc2cc1OCCCN1CCOCC1]

Foretinib (GSK1363089) has previously demonstrated potential antineoplastic activity. Prior to the instant invention, foretinib has shown binding to and selective inhibition of hepatocyte growth factor (HGF) receptor c-MET and vascular endothelial growth factor receptor 2 (VEGFR2), which may result in the inhibition of tumor angiogenesis, tumor cell proliferation and metastasis. Foretinib was not known as a CA-IV binder or a shuttle for crossing the BBB relying on CA-IV activity. It was unexpectedly discovered that foretinib is a CA-IV binder. The invention beneficially recognizes that this property of foretinib may be utilized in developing conjugates for crossing the BBB for delivery of therapeutic cargo.

Tanespimycin (17-AAG) is a 19-membered macrocyle that is geldanamycin in which the methoxy substituent attached to the benzoquinone moiety has been replaced by an allylamino group —C₃₁H₄₃N₃O₈; [(4E,6Z,8S,9S,10E,12S,13R,14S,16R)-13-hydroxy-8,14-dimethoxy-4,10,12,16-tetramethyl-3,20,22-trioxo-19-(prop-2-enylamino)-2-azabicyclo[16.3.1]docosa-1(21),4,6,10,18-pentaen-9-yl]carbamate—having the structure:

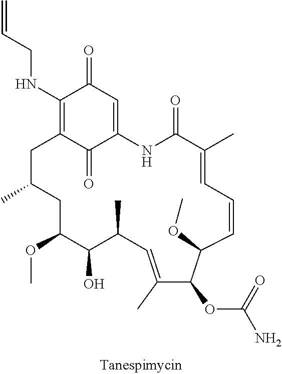 [SMILES: C=CCNC1=C2C[C@@H](C)C[C@H](OC)[C@H](O)[C@@H](C)/C=C(\C)[C@H](OC(N)=O)[C@@H](OC)/C=C\C=C(/C)C(=O)NC(=CC1=O)C2=O]

Prior to the instant invention, tanespimycin has been shown to be a potent inhibitor of heat shock protein 90 (Hsp90). A less toxic analogue than geldanamycin, it induces apoptosis and displays antitumour effects. It has a role as an antineoplastic agent, a Hsp90 inhibitor and an apoptosis inducer. It is a secondary amino compound, an ansamycin, a carbamate ester, an organic heterobicyclic compound and a member of 1,4-benzoquinones. It is functionally related to a geldanamycin. Tanespimycin was not known as a CA-IV binder or a shuttle for crossing the BBB relying on CA-IV activity. It was unexpectedly discovered that tanespimycin is a CA-IV binder. The invention beneficially recognizes that this property of tanespimycin may be utilized in developing conjugates for crossing the BBB for delivery of therapeutic cargo.

Nilotinib (AMN-107) is a member of (trifluoromethyl)benzenes —C₂₈H₂₂F3N₇O; 4-methyl-N-[3-(4-methylimidazol-1-yl)-5-(trifluoromethyl)phenyl]-3-[(4-pyridin-3-ylpyrimidin-2-yl)amino]benzamide—having the structure:

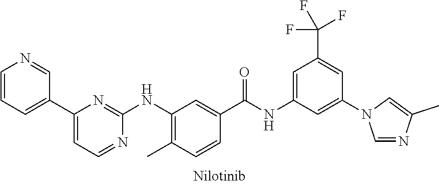 [SMILES: Cc1cn(-c2cc(NC(=O)c3ccc(C)c(Nc4nccc(-c5cccnc5)n4)c3)cc(C(F)(F)F)c2)cn1]

Nilotinib has a role as an antineoplastic agent, a tyrosine kinase inhibitor and an anticoronaviral agent. Nilotinib has been shown to be a Kinase Inhibitor. Prior to the instant invention, nilotinib was shown as a Bcr-Abl Tyrosine Kinase Inhibitor, Cytochrome P450 2C8 Inhibitor, Cytochrome P450 2D6 Inhibitor, Cytochrome P450 2B6 Inducer, Cytochrome P450 2C8 Inducer, UGT1A1 Inhibitor, and P-Glycoprotein Inhibitor. Nilotinib was not known as a CA-IV binder or a shuttle for crossing the BBB relying on CA-IV activity. It was unexpectedly discovered that nilotinib is a CA-IV binder. The invention beneficially recognizes that this property of nilotinib may be utilized in developing conjugates for crossing the BBB for delivery of therapeutic cargo.

Tozasertib (VX-680; MK-0457) is an aurora kinase inhibitor —C₂₃H₂₈N₈OS; N-[4-[[4-(4-methyl-1-piperazinyl)-6-[(5-methyl-1H-pyrazol-3-yl)amino]-2-pyrimidinyl]thio]phenyl]cyclopropanecarboxamide—having the structure:

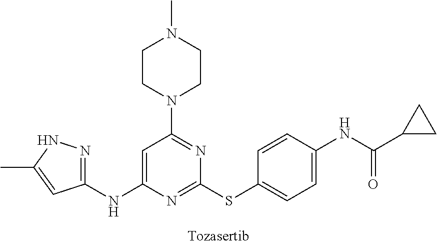 [SMILES: Cc1cc(Nc2cc(N3CCN(C)CC3)nc(Sc3ccc(NC(=O)C4CC4)cc3)n2)n[nH]1]

Prior to instant invention, tozasertib was shown to bind to and inhibit Aurora kinases (AKs), thereby inducing apoptosis in tumor cells in which AKs are overexpressed. AKs, a family of serine-threonine kinases, are essential for mitotic progression, spindle formation, centrosome maturation, chromosomal segregation, and cytokinesis. Tozasertib was not known as a CA-IV binder or a shuttle for crossing the BBB relying on CA-IV activity. It was unexpectedly discovered that tozasertib is a CA-IV binder. The invention beneficially recognizes that this property of tozasertib may be utilized in developing conjugates for crossing the BBB for delivery of therapeutic cargo.

Ivacaftor (VX-770) is an aromatic amide obtained by formal condensation of the carboxy group of 4-oxo-1,4-dihydroquinoline-3-carboxylic acid with the amino group of 5-amino-2,4-di-tert-butylphenol. Ivacaftor is described by the molecular formula C₂₄H₂₈N₂O₃ and IUPAC name N-(2,4-ditert-butyl-5-hydroxyphenyl)-4-oxo-1H-quinoline-3-carboxamide, with the structure:

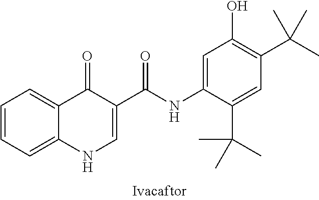 [SMILES: CC(C)(C)c1cc(C(C)(C)C)c(NC(=O)c2c[nH]c3ccccc3c2=O)cc1O]

Ivacaftor has been shown to have a role as a CFTR potentiator and an orphan drug for cystic fibrosis. Ivacaftor is a quinolone, a member of phenols, an aromatic amide and a monocarboxylic acid amide. Prior to the instant invention, ivacaftor was shown to be a Chloride Channel Activation Potentiator, Cytochrome P450 2C9 Inhibitor, P-Glycoprotein Inhibitor, and Cytochrome P450 3A Inhibitor. Ivacaftor was not known as a CA-IV binder or a shuttle for crossing the BBB relying on CA-IV activity. It was unexpectedly discovered that ivacaftor is a CA-IV binder. The invention beneficially recognizes that this property of ivacaftor may be utilized in developing conjugates for crossing the BBB for delivery of therapeutic cargo.

Linsitinib (OSI-906) is an orally bioavailable small molecule inhibitor of the insulin-like growth factor 1 receptor (IGF-1R) with potential antineoplastic activity. Linsitinib is described by the molecular formula C₂₆H₂₃N₅O and IUPAC name 3-[8-amino-1-(2-phenylquinolin-7-yl)imidazo[1,5-a]pyrazin-3-yl]-1-methylcyclobutan-1-ol, with the structure:

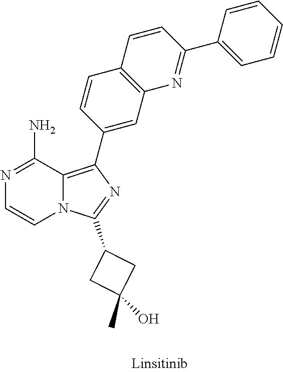 [SMILES: C[C@]1(O)C[C@@H](c2nc(-c3ccc4ccc(-c5ccccc5)nc4c3)c3c(N)nccn32)C1]

Prior to the instant invention, linsitinib was shown to selectively inhibit IGF-1R, which may result in the inhibition of tumor cell proliferation and the induction of tumor cell apoptosis. Overexpressed in a variety of human cancers, IGF-1R stimulates cell proliferation, enables oncogenic transformation, and suppresses apoptosis. Linsitinib was not known as a CA-IV binder or a shuttle for crossing the BBB relying on CA-IV activity. It was unexpectedly discovered that linsitinib is a CA-IV binder. The invention beneficially recognizes that this property of linsitinib may be utilized in developing conjugates for crossing the BBB for delivery of therapeutic cargo.

Epirubicin hydrochloride is the hydrochloride salt of the 4′-epi-isomer of the anthracycline antineoplastic antibiotic doxorubicin. Epirubicin Hydrochloride is described by the molecular formula C₂₇H₃₀ClNO₁₁ and IUPAC name (7S,9S)-7-[(2R,4S,5R,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione;hydrochloride. Epirubicin has the structure:

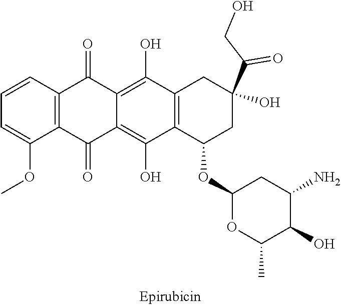 [SMILES: COc1cccc2c1C(=O)c1c(O)c3c(c(O)c1C2=O)C[C@@](O)(C(=O)CO)C[C@@H]3O[C@H]1C[C@H](N)[C@@H](O)[C@H](C)O1]

Prior to the instant invention, epirubicin was shown to intercalate into DNA and inhibit topoisomerase II, thereby inhibiting DNA replication and ultimately, interfering with RNA and protein synthesis. This agent also produces toxic free-radical intermediates and interacts with cell membrane lipids causing lipid peroxidation. Epirubicin was not known as a CA-IV binder or a shuttle for crossing the BBB relying on CA-IV activity. It was unexpectedly discovered that epirubicin is a CA-IV binder. The invention beneficially recognizes that this property of epirubicin may be utilized in developing conjugates for crossing the BBB for delivery of therapeutic cargo.

Zosuquidar trihydrochloride (Ly335979) is a difluorocyclopropyl quinoline. Zosuquidar trihydrochloride is described by the molecular formula C₃₂H₃₄Cl₃F₂N₃O₂ and IUPAC name (2R)-1-[4-[(2S,4R)-3,3-difluoro-11-tetracyclo[10.4.0.0^{2,4}.0^{5,10}]hexadeca-1(16),5,7,9,12,14-hexaenyl]piperazin-1-yl]-3-quinolin-5-yloxypropan-2-ol;trihydrochloride. Zosuquidar has the structure:

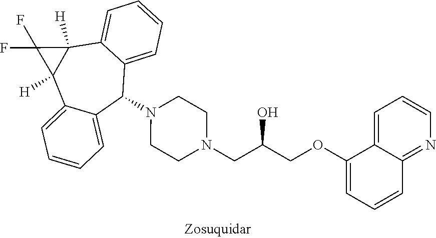 [SMILES: O[C@@H](COc1cccc2ncccc12)CN1CCN([C@@H]2c3ccccc3[C@@H]3[C@H](c4ccccc42)C3(F)F)CC1]

Prior to the instant invention, zosuquidar trihydrochloride was shown to bind with high affinity to P-glycoprotein and inhibit P-glycoprotein-mediated multidrug resistance (MDR). P-glycoprotein, encoded by the MDR-1 gene, is a member of the ATP-binding cassette superfamily of transmembrane transporters and prevents the intracellular accumulation of many natural product-derived cytotoxic agents. (NCI04). Zosuquidar was not known as a CA-IV binder or a shuttle for crossing the BBB relying on CA-IV activity. It was unexpectedly discovered that zosuquidar is a CA-IV binder. The invention beneficially recognizes that this property of zosuquidar may be utilized in developing conjugates for crossing the BBB for delivery of therapeutic cargo.

Ziprasidone hydrocholoride is the hydrochloride salt form of ziprasidone, a benzothiazolylpiperazine derivative and an atypical antipsychotic agent with an antischizophrenic property. Ziprasidone hydrocholoride is described by the molecular formula C₂₁H₂₂Cl₂N₄OS and IUPAC name 5-[2-[4-(1,2-benzothiazol-3-yl)piperazin-1-yl]ethyl]-6-chloro-1,3-dihydroindol-2-one;hydrochloride. Ziprasidone has the structure:

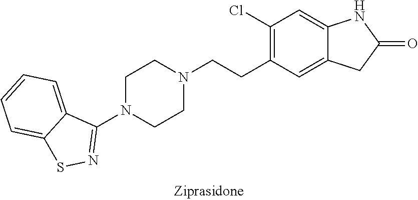 [SMILES: O=C1Cc2cc(CCN3CCN(c4nsc5ccccc45)CC3)c(Cl)cc2N1]

Prior to the instant invention, ziprasidone hydrochloride was shown to function as an antagonist at the dopamine D2 and serotonin 5-HT2A and 5-HT1D receptors, and as an agonist at the 5-HT1A receptor. Ziprasidone hydrochloride also inhibits synaptic reuptake of serotonin and norepinephrine. The mechanism of action by which ziprasidone hydrochloride exerts its antischizophrenic effect is unknown but is potentially mediated through a combination of dopamine D2 and serotonin 5-HT2 antagonism. This agent also has antagonistic activity against histamine H1 and alpha-1-adrenergic receptors. Ziprasidone was not known as a CA-IV binder or a shuttle for crossing the BBB relying on CA-IV activity. It was unexpectedly discovered that ziprasidone is a CA-IV binder. The invention beneficially recognizes that this property of ziprasidone may be utilized in developing conjugates for crossing the BBB for delivery of therapeutic cargo.

Acetylcysteine, also known as N-Acetyl-L-cysteine (NAC) is an N-acetyl-L-amino acid that is the N-acetylated derivative of the natural amino acid L-cysteine. Acetylcysteine is described by the molecular formula C₅H₉NO₃S and IUPAC name (2R)-2-acetamido-3-sulfanylpropanoic acid. Acetylcysteine has the structure:

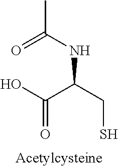

Acetylcysteine has a role as an antiinfective agent, antioxidant, antiviral drug, antidote to paracetamol poisoning, a vulnerary, a mucolytic, a human metabolite, a radical scavenger, a ferroptosis inhibitor, and a geroprotector. Acetylcysteine was granted U.S. Food and Drug Administration (FDA) approval on Sep. 14, 1963, and has also been studied for a wide variety of off-label indications. Acetylcysteine was not known as a CA-IV binder or a shuttle for crossing the BBB relying on CA-IV activity. It was unexpectedly discovered that acetylcysteine is a CA-IV binder. The invention beneficially recognizes that this property of acetylcysteine may be utilized in developing conjugates for crossing the BBB for delivery of therapeutic cargo.

Zafirlukast is an oral leukotriene receptor antagonist (LTRA) for the maintenance treatment of asthma, often used in conjunction with an inhaled steroid and/or long-acting bronchodilator. Zafirlukast is described by the molecular formula C₃₁H₃₃N₃O₆S and IUPAC name cyclopentyl N-[3-[[2-methoxy-4-[(2-methylphenyl)sulfonylcarbamoyl]phenyl]methyl]-1-methylindol-5-yl]carbamate. Zafirlukast has the structure:

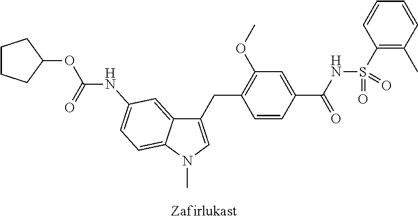 [SMILES: COc1cc(C(=O)NS(=O)(=O)c2ccccc2C)ccc1Cc1cn(C)c2ccc(NC(=O)OC3CCCC3)cc12]

Prior to the instant invention, Zafirlukast was shown to block the action of the cysteinyl leukotrienes on the CysLT1 receptors, thus reducing constriction of the airways, build-up of mucus in the lungs and inflammation of the breathing passages. Zafirlukast was also shown to be a Cytochrome P450 2C9 Inhibitor. Zafirlukast was not known as a CA-IV binder or a shuttle for crossing the BBB relying on CA-IV activity. It was unexpectedly discovered that zafirlukast is a CA-IV binder. The invention beneficially recognizes that this property of zafirlukast may be utilized in developing conjugates for crossing the BBB for delivery of therapeutic cargo.

Elvitegravir (GS-9137; JTK-303) is a quinolinemonocarboxylic acid that is 7-methoxy-4-oxo-1,4-dihydroquinoline-3-carboxylic acid substited at position 1 by a 1-hydroxy-3-methylbutan-2-yl group and at position 6 by a 3-chloro-2-fluorobenzyl group (the S-enantiomer). Elvitegravir is described by the molecular formula C₂₃H₂₃ClFNO₅ and IUPAC name 6-[(3-chloro-2-fluorophenyl)methyl]-1-[(2S)-1-hydroxy-3-methylbutan-2-yl]-7-methoxy-4-oxoquinoline-3-carboxylic acid. Elvitegravir has the structure:

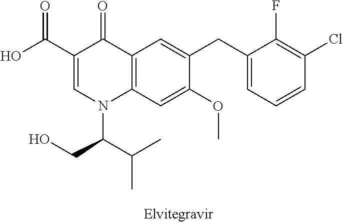 [SMILES: COc1cc2c(cc1Cc1cccc(Cl)c1F)c(=O)c(C(=O)O)cn2[C@H](CO)C(C)C]

Prior to the instant invention, Elvitegravir was shown to be a human immunodeficiency virus type 1 (HIV-1) integrase strand transfer inhibitor (INSTI) used for the treatment of HIV-1 infection in antiretroviral treatment-experienced adults. Elvitegravir was not known as a CA-IV binder or a shuttle for crossing the BBB relying on CA-IV activity. It was unexpectedly discovered that elvitegravir is a CA-IV binder. The invention beneficially recognizes that this property of elivtegravir may be utilized in developing conjugates for crossing the BBB for delivery of therapeutic cargo.

Meclizine dihydrochloride (BONINE) is a histamine H1 antagonist used in the treatment of motion sickness, vertigo, and nausea during pregnancy and radiation sickness. Meclizine dihydrochloride is described by the molecular formula C₂₅H₂₉Cl₃N₂ and IUPAC name 1-[(4-chlorophenyl)-phenylmethyl]-4-[(3-methylphenyl)methyl]piperazine;dihydrochloride. Meclizine has the structure:

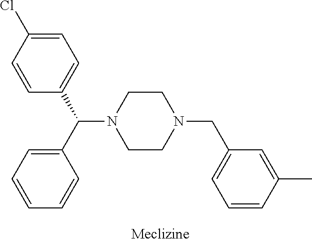 [SMILES: Cc1cccc(CN2CCN([C@H](c3ccccc3)c3ccc(Cl)cc3)CC2)c1]

Prior to the instant invention, meclizine was shown to be an antagonist at H1 receptors and possesses anticholinergic, central nervous system depressant, and local anesthetic effects. Meclizine was patented in 1951 and came into medical use in 1953. Its antiemetic and antivertigo effects are not fully understood, but its central anticholinergic properties are partially responsible. The drug depresses labyrinth excitability and vestibular stimulation, and it may affect the medullary chemoreceptor trigger zone. Meclizine was not known as a CA-IV binder or a shuttle for crossing the BBB relying on CA-IV activity. It was unexpectedly discovered that meclizine is a CA-IV binder. The invention beneficially recognizes that this property of meclizine may be utilized in developing conjugates for crossing the BBB for delivery of therapeutic cargo.

Candesartan cilexetil (ATACAND) is a synthetic, benzimidazole-derived angiotensin II receptor antagonist prodrug with antihypertensive activity. Candesartan cilexetil is described by the molecular formula C₃₃H₃₄N₆O₆ and IUPAC name 1-cyclohexyloxycarbonyloxyethyl 2-ethoxy-3-[[4-[2-(2H-tetrazol-5-yl)phenyl]phenyl]methyl]benzimidazole-4-carboxylate. Candesartan and candesartan cilexetil have the following structures:

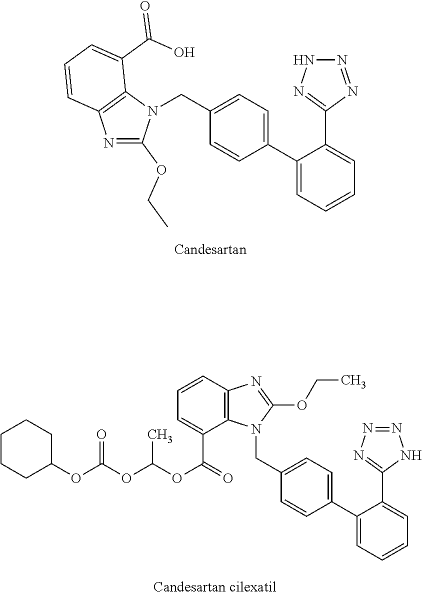 [SMILES: CCOc1nc2cccc(C(=O)O)c2n1Cc1ccc(-c2ccccc2-c2nn[nH]n2)cc1 ; CCOc1nc2cccc(C(=O)OC(C)OC(=O)OC3CCCCC3)c2n1Cc1ccc(-c2ccccc2-c2nnn[nH]2)cc1]

Candesartan is administered orally as the prodrug, candesartan cilexetil, which is rapidly converted to its active metabolite, candesartan, during absorption in the gastrointestinal tract. After hydrolysis of candesartan cilexetil to candesartan during gastrointestinal absorption, candesartan selectively competes with angiotensin II for the binding of the angiotensin II receptor subtype 1 (AT1) in vascular smooth muscle, blocking angiotensin II-mediated vasoconstriction and inducing vasodilatation. In addition, antagonism of ATl in the adrenal gland inhibits angiotensin II-stimulated aldosterone synthesis and secretion by the adrenal cortex; sodium and water excretion increase, followed by a reduction in plasma volume and blood pressure. Candesartan was patented in 1990 and approved for medical use in 1997. Candesartan was not known as a CA-IV binder or a shuttle for crossing the BBB relying on CA-IV activity. It was unexpectedly discovered that candesartan is a CA-IV binder. The invention beneficially recognizes that this property of candesartan may be utilized in developing conjugates for crossing the BBB for delivery of therapeutic cargo.

Diclazuril (CLINACOX, VEXOCAN, PORTAZIL) is a synthetic benzenacetonitrile derivative, belonging to the asymmetric triazines. Its mode of action is not precisely known, however, it interrupts the life cycle of eimerians. Diclazuril is described by the molecular formula C₁₇H₉Cl₃N₄O₂ and IUPAC name 2-(4-chlorophenyl)-2-[2,6-dichloro-4-(3,5-dioxo-1,2,4-triazin-2-yl)phenyl]acetonitrile. Diclazuril has the structure:

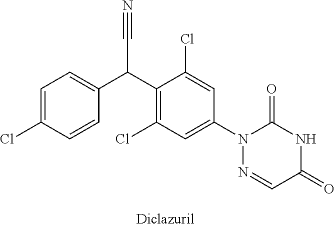 [SMILES: N#CC(c1ccc(Cl)cc1)c1c(Cl)cc(-n2ncc(=O)[nH]c2=O)cc1Cl]

Diclazuril was not known as a CA-IV binder or a shuttle for crossing the BBB relying on CA-IV activity. It was unexpectedly discovered that diclazuril is a CA-IV binder. The invention beneficially recognizes that this property of diclazuril may be utilized in developing conjugates for crossing the BBB for delivery of therapeutic cargo.

Flunarizine dihydrochloride is the chloride salt of flunarizine. Flunarizine dihydrochloride is described by the molecular formula C₂₆H₂₈Cl₂F₂N₂ and IUPAC name 1-[bis(4-fluorophenyl)methyl]-4-[(E)-3-phenylprop-2-enyl]piperazine;dihydrochloride. Flunarizine (sold under the trade name SIBELIUM) has the structure:

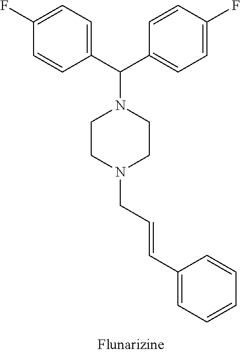 [SMILES: Fc1ccc(C(c2ccc(F)cc2)N2CCN(C/C=C/c3ccccc3)CC2)cc1]

Flunarizine dihydrochloride is a diarylmethane, with flunarizine, prior to the instant invention, being shown to be a selective calcium entry blocker with calmodulin binding properties and histamine H1 blocking activity. It is effective in the prophylaxis of migraine, occlusive peripheral vascular disease, vertigo of central and peripheral origin, and as an adjuvant in the therapy of epilepsy. Flunarizine was not known as a CA-IV binder or a shuttle for crossing the BBB relying on CA-IV activity. It was unexpectedly discovered that flunarizine is a CA-IV binder. The invention beneficially recognizes that this property of flunarizine may be utilized in developing conjugates for crossing the BBB for delivery of therapeutic cargo.

Evacetrapib (LY2484595) is a benzazepine,—chemical formula C₃₁H₃₆F₆N₆O₂; 4-[[(5S)-5-[[3,5-bis(trifluoromethyl)phenyl]methyl-(2-methyltetrazol-5-yl)amino]-7,9-dimethyl-2,3,4,5-tetrahydro-1-benzazepin-1-yl]methyl]cyclohexane-1-carboxylic acid—having the structure:

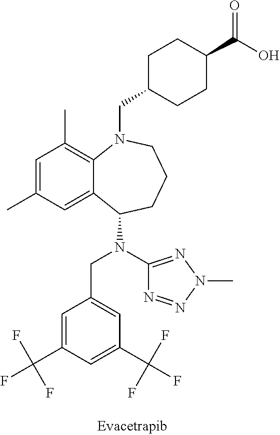 [SMILES: Cc1cc(C)c2c(c1)[C@@H](N(Cc1cc(C(F)(F)F)cc(C(F)(F)F)c1)c1nnn(C)n1)CCCN2C[C@H]1CC[C@H](C(=O)O)CC1]

Prior to the instant invention, evacetrapib was shown to inhibit cholesterylester transfer protein (CETP). However, evacetrapib's evaluation for treatment of high-risk vascular disease was discontinued due to lack of efficacy, due to increased deaths and little identifiable cardiovascular benefit. Evacetrapib was not known as a CA-IV binder or a shuttle for crossing the BBB relying on CA-IV activity. It was unexpectedly discovered that evacetrapib is a CA-IV binder. The invention beneficially recognizes that this property of evacetrapib may be utilized in developing conjugates for crossing the BBB for delivery of therapeutic cargo.

Estradiol valerate (EV or E2V) is a pro-drug ester of the steroid hormone estradiol, that circulates endogenously within the human body. Estradiol valerate is described by the molecular formula C₂₃H₃₂O₃ and IUPAC name [(8R,9S,13S,14S,17S)-3-hydroxy-13-methyl-6,7,8,9,11,12,14,15,16,17-decahydrocyclopenta[a]phenanthren-17-yl]pentanoate. Estradiol valerate has the structure:

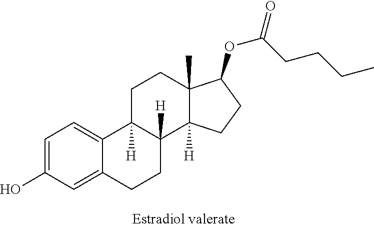

Estradiol valerate is an estrogen and agonist of the estrogen receptor. Estradiol valerate sold for use by mouth under the brand name PROGYNOVA and for use by injection under the brand names DELESTROGEN and PROGYNON DEPOT. Prior to the instant invention, it has been used in hormone therapy for menopausal symptoms and low estrogen levels, hormone therapy for transgender people, and in hormonal birth control. It is also used in the treatment of prostate cancer. Estradiol valerate was first described in 1940 and was introduced for medical use in 1954. Estradiol was not known as a CA-IV binder or a shuttle for crossing the BBB relying on CA-IV activity. It was unexpectedly discovered that estradiol is a CA-IV binder. The invention beneficially recognizes that this property of estradiol may be utilized in developing conjugates for crossing the BBB for delivery of therapeutic cargo.

Etravirine (TMC125; INTELENCE) is an aminopyrimidine that consists of 2,6-diaminopyrimidine bearing a bromo substituent at position 5, a 4-cyano-2,6-dimethylphenoxy substituent at position 4 and having a 4-cyanophenyl substituent attached to the 2-amino group. Etravirine is described by the molecular formula C₂₀H₁₅BrN6O and IUPAC name 4-[6-amino-5-bromo-2-(4-cyanoanilino)pyrimidin-4-yl]oxy-3,5-dimethylbenzonitrile. Etravirine has the structure:

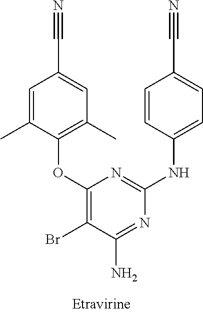 [SMILES: Cc1cc(C#N)cc(C)c1Oc1nc(Nc2ccc(C#N)cc2)nc(N)c1Br]

Prior to the instant invention, etravirine was shown to be an antiretroviral agent, specifically classified as a Non-Nucleoside Reverse Transcriptase Inhibitor (NNRTI). Etraverine is used clinically for the treatment of human immunodeficiency virus type 1 (HIV-1) infection, with NNRTI of HIV-1 binding directly to reverse transcriptase and blocking RNA-dependent and DNA-dependent DNA polymerase activities. Etravirine was not known as a CA-IV binder or a shuttle for crossing the BBB relying on CA-IV activity. It was unexpectedly discovered that etravirine is a CA-IV binder. The invention beneficially recognizes that this property of etravirine may be utilized in developing conjugates for crossing the BBB for delivery of therapeutic cargo.

Montelukast sodium is the orally bioavailable monosodium salt of montelukast, a selective cysteinyl leukotriene receptor antagonist with anti-inflammatory and bronchodilating activities. Montelukast is described by the molecular formula C₃₅H₃₅ClNNaO₃S and IUPAC name sodium;2-[1-[[(1R)-1-[3-[(E)-2-(7-chloroquinolin-2-yl)ethenyl]phenyl]-3-[2-(2-hydroxypropan-2-yl)phenyl]propyl]sulfanylmethyl]cyclopropyl]acetate. Montelukast (sold under the trade name SINGULAIR) has the structure:

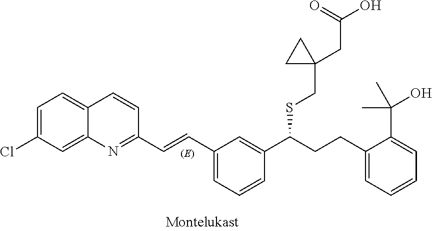 [SMILES: CC(C)(O)c1ccccc1CC[C@@H](SCC1(CC(=O)O)CC1)c1cccc(/C=C/c2ccc3ccc(Cl)cc3n2)c1]

Prior to the instant invention, montelukast was shown to selectively and competitively block the cysteinyl leukotriene 1 (CysLT1) receptor, preventing binding of the inflammatory mediator leukotriene D4 (LTD4) for the treatment of asthma, exercise induced bronchospasm, allergic rhinitis, and urticaria. Montelukast was approved for medical use in the United States in 1998. Montelukast was not known as a CA-IV binder or a shuttle for crossing the BBB relying on CA-IV activity. It was unexpectedly discovered that montelukast is a CA-IV binder. The invention beneficially recognizes that this property of montelukast may be utilized in developing conjugates for crossing the BBB for delivery of therapeutic cargo.

Nelfinavir mesylate is a methanesulfonate (mesylate) salt prepared from equimolar amounts of nelfinavir and methanesulfonic acid. Nelfinavir mesylate is described by the molecular formula C₃₃H₄₉N₃O₇S₂ and IUPAC name (3S,4aS,8aS)—N-tert-butyl-2-[(2R,3R)-2-hydroxy-3-[(3-hydroxy-2-methylbenzoyl)amino]-4-phenylsulfanylbutyl]-3,4,4a,5,6,7,8,8a-octahydro-1H-isoquinoline-3-carboxamide;methanesulfonic acid. Nelfinavir (sold under the trade name VIRACEPT) has the structure:

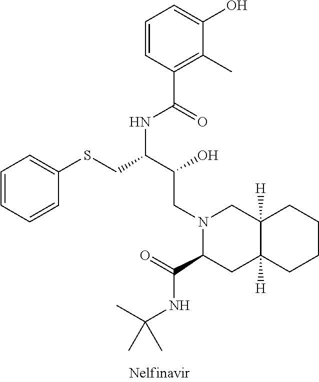 [SMILES: Cc1c(O)cccc1C(=O)N[C@@H](CSc1ccccc1)[C@H](O)CN1C[C@H]2CCCC[C@H]2C[C@H]1C(=O)NC(C)(C)C]

Nelfinavir mesylate is a mesylate salt form of nelfinavir, a synthetic protease inhibitor that selectively binds to and inhibits human immunodeficiency virus (HIV) protease. Nelfinavir has activity against HIV-1 and HIV-2 proteases. Nelafinavir was not known as a CA-IV binder or a shuttle for crossing the BBB relying on CA-IV activity. It was unexpectedly discovered that nelfinavir is a CA-IV binder. The invention beneficially recognizes that this property of nelfinavir may be utilized in developing conjugates for crossing the BBB for delivery of therapeutic cargo.

Mefloquine hydrochloride is the hydrochloride salt form of mefloquine, a piperidinyl quinidine with antimalarial activity. Mefloquine hydrochloride is described by the molecular formula C₁₇H₁₇ClF₆N₂O and IUPAC name (S)-[2,8-bis(trifluoromethyl)quinolin-4-yl]-[(2R)-piperidin-2-yl]methanol;hydrochloride. Mefloquine (sold under the trade name LARIAM) is a chiral molecule with two asymmetric carbon centers, having four different stereoisomers. The drug is currently manufactured and sold as a racemate of the (R,S)- and (S,R)-enantiomers with the structure:

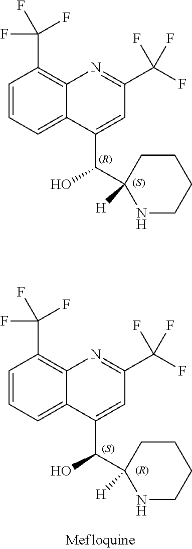

Although the exact mechanism of mefloquine hydrochloride is largely unknown, mefloquine hydrochloride is thought to act as a blood schizonticide and may exert its actions by interacting with the phospholipid bilayer, thereby interfering with the stability of the cell membrane and causing cell lysis. Mefloquine hydrochloride is active against Plasmodium falciparum and Plasmodium vivax. Mefloquine was not known as a CA-IV binder or a shuttle for crossing the BBB relying on CA-IV activity. It was unexpectedly discovered that mefloquine is a CA-IV binder. The invention beneficially recognizes that this property of mefloquine may be utilized in developing conjugates for crossing the BBB for delivery of therapeutic cargo.

Pimozide (ORAP) is a member of the class of benzimidazoles that is 1,3-dihydro-2H-benzimidazol-2-one in which one of the nitrogens is substituted by a piperidin-4-yl group, which in turn is substituted on the nitrogen by a 4,4-bis(p-fluorophenyl)butyl group. Pimozide is described by the molecular formula C₂₈H₂₉F₂N₃O and IUPAC name 3-[1-[4,4-bis(4-fluorophenyl)butyl]piperidin-4-yl]-1H-benzimidazol-2-one. Pimozide has the structure:

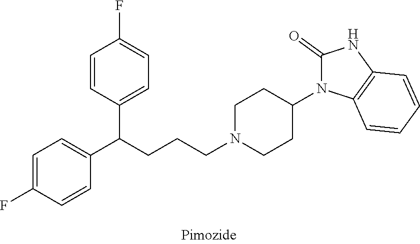 [SMILES: O=c1[nH]c2ccccc2n1C1CCN(CCCC(c2ccc(F)cc2)c2ccc(F)cc2)CC1]

Prior to the instant invention, pimozide was shown to have a role as an Hi-receptor antagonist, a serotonergic antagonist, a first generation antipsychotic, an antidyskinesia agent and a dopaminergic antagonist. Pimozide acts as an antagonist of the D2, D3, and D4 receptors and the 5-HT7 receptor and is also a hERG blocker. In 1985 pimozide was approved by the FDA for marketing as an orphan drug for the treatment of Tourette's syndrome. Pimozide was not known as a CA-IV binder or a shuttle for crossing the BBB relying on CA-IV activity. It was unexpectedly discovered that pimozide is a CA-IV binder. The invention beneficially recognizes that this property of pimozide may be utilized in developing conjugates for crossing the BBB for delivery of therapeutic cargo.

Zolpidem (AMBIEN) is an imidazo[1,2-a]pyridine compound having a 4-tolyl group at the 2-position, an N,N-dimethylcarbamoylmethyl group at the 3-position and a methyl substituent at the 6-position. It has a role as a central nervous system depressant, a GABA agonist and a sedative. Zolpidem is described by the molecular formula C₁₉H₂₁N₃O and IUPAC name N,N-dimethyl-2-[6-methyl-2-(4-methylphenyl)imidazo[1,2-a]pyridin-3-yl]acetamide. Zolpidem has the structure:

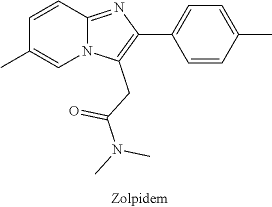

Zolpidem is a ligand of high-affinity positive modulator sites of GABA_{A} receptors, which enhances GABAergic inhibition of neurotransmission in the central nervous system. It selectively binds to α1 subunits of the pentameric ion channel. Zolpidem has about 10-fold lower affinity for the α2- and α3-subunits than for α1, and no appreciable affinity for α5 subunit-containing receptors. Zolpidem was initially approved by the FDA in 1992 and is primarily used for the short-term treatment of sleeping problems. Zolpidem was not known as a CA-IV binder or a shuttle for crossing the BBB relying on CA-IV activity. It was unexpectedly discovered that zolpidem is a CA-IV binder. The invention beneficially recognizes that this property of zolpidem may be utilized in developing conjugates for crossing the BBB for delivery of therapeutic cargo.

Hydroxyprogesterone caproate (DELALUTIN) is a synthetic pregnane corticosteroid hormone, and an ester derivative of 17α-hydroxyprogesterone formed from caproic acid (hexanoic acid). Hydroxyprogesterone caproate is described by the molecular formula C₂₇H₄₀O₄ and IUPAC name [(8R,9S,10R,13S,14S,17R)-17-acetyl-10,13-dimethyl-3-oxo-2,6,7,8,9,11,12,14,15,16-decahydro-1H-cyclopenta[a]phenanthren-17-yl]hexanoate. Hydroxyprogesterone caproate has the structure:

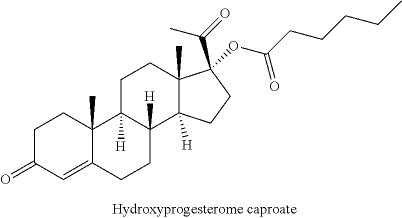

Prior to the instant invention, hydroxyprogesterone caproate was shown to have progestogenic activity, some antimineralocorticoid activity, and limited other hormonal activity. Hydroxyprogesterone caproate was first approved by the FDA in 1956, used to reduce the risk of preterm birth in women pregnant with one baby who have a history of spontaneous preterm birth. The medication was discontinued in the United States in 1999 and subsequently reintroduced in the United States under the brand name MAKENA for the treatment of preterm birth in 2011. The approvals of MAKENA and its generics were withdrawn by the FDA in April 2023. Hydroxyprogesterone was not known as a CA-IV binder or a shuttle for crossing the BBB relying on CA-IV activity. It was unexpectedly discovered that hydroxyprogesterone is a CA-IV binder. The invention beneficially recognizes that this property of hydroxyprogesterone may be utilized in developing conjugates for crossing the BBB for delivery of therapeutic cargo.

### Small Molecule Derivative

Aspects of the invention include derivatives of small molecules, said derivatives capable of facilitating the delivery of a pharmaceutical agent across the BBB. Non-limiting moieties that may be used to produce a derivate are described in detail below. Such a derivative would have biological activity similar to the parent molecule.

For example, derivatives may result from the addition or substitution of an existing group. In certain instances the derivatives may comprise substitution by alkyl or halogen. Derivates may comprise the substitution or addition of one or more of a variety of groups selected from, but not limited to: —OR′, ═P, =NR′, =N—OR′, —NR′R″—SR′, -halogen, —SiR′R″R″, —OC(O)R, —C(O)R, —CO2R —C(O)NR′R″, —OC(O)NR′R″, —NR″C(O)R, —NR′—C(O)NR″R′″, —NR″C(O)OR′, —NR—C(NR′R″)═NR″′, —S(O)R, —S(O)₂R′, —S(O)₂NR′R″, —NRSO2R′, —CN, CF₃, fluorinated C₁-C₄ alkyl, and —NO₂ in a number ranging from zero to (2m′+1), where m′ is the total number of carbon atoms in such groups. R′, R″, R″′ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. Other non-limiting examples of substituents include (C1-C6)alkyl, (C2-C8)alkenyl, (C3-C8)alkynyl, halogen, halo(C1-C6)alkyl, hydroxy, —O(C1-C6)alkyl, halo(C1-C6)alkoxy, (C3-C8)cycloalkyl, (C6-C10)aryl, heterocyclyl, heteroaryl, amino, cyano, nitro, (C1-C6)alkyl-OH, (C1-C6)alkyl-O—(C1-C6)alkyl, (C1-C6)alkyl(C6-C10)aryl, —C(O)(C1-C6)alkyl, —C(O)NR′R″, —S(O)(C1-C6)alkyl, —S(O)NR′R″, —S(O)2(C1-C6)alkyl, —S(O)2NR′R″, —O(C1-C6)alkyl-S(O)(C1-C6)alkyl, —O(C1-C6)alkyl-S(O)NR′R″, —O(C1-C6)alkyl-S(O)2(C1-C6)alkyl, and —O(C1-C6)alkyl-S(O)₂NR′R″.

Unless otherwise stated, the moieties may be optionally substituted, i.e., they may be substituted at one or more positions. The terms substituted, whether preceded by the term “optionally” or not, and substituent, as used herein, refer to the ability to change one or more functional groups for another functional group or groups on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions). Substitutions include, but are not limited to, hydrogen, alkyls, cycloalkyls, halogens, hydroxy groups, and oxo groups.

When the term “independently selected” is used, the substituents being referred to (e.g., R groups, such as groups R1, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein.

The term hydrocarbon, as used herein, refers to any chemical group comprising hydrogen and carbon. The hydrocarbon may be substituted or unsubstituted. As would be known to one skilled in the art, all valences must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, ally 1, vinyl, n-butyl, tert-butyl, ethynyl, cyclohexyl, and the like.

The term “alkyl” by itself or as part of another substituent, means, unless otherwise stated, a straight (i.e., unbranched) or branched chain, acyclic or cyclic saturated hydrocarbon group, or combination thereof, and can include di- and multivalent groups, having the number of carbon atoms designated (e.g., C₁-C₁₀ means one to ten carbons, including 1, 2, 3, 4, 5, 6, 7, 8, 9, and 10 carbons). In particular embodiments, the term “alkyl” refers to C1-20 inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic saturated hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologues and isomers thereof.

“Branched” refers to an alkyl group in which a lower alkyl group, such as methyl, ethyl, or propyl, is attached to a linear alkyl chain. “Lower alkyl” refers to an alkyl group having 1 to about 8 carbon atoms (i.e., a C1-8 alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 15 carbon atoms or heteroatoms, or combinations thereof, consisting of at least one carbon atom and at least one heteroatom, such as O, N, P, Si or S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CHO—CH₃, —Si(CH₃)3, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′.

“Cycloalkyl” refers to a saturated monocyclic or multicyclic ring system of from about 3 to about 15 carbon atoms, e.g., 3, 4, 5, 6, 7, 8, 9, or 10 carbon atoms. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Examples of cycloalkyl include, but are not limited to, cyclopentyl, cyclohexyl, 1-cyclohexenyl, 3-cyeiohexenyl, cycloheptyl, and the like.

The term “cycloalkylalkyl,” as used herein, refers to a cycloalkyl group as defined above, which is attached to the parent molecular moiety through an alkylene moiety, also as defined above, e.g., a C1-20 alkylene moiety. Examples of cycloalkylalkyl groups include cyclopropylmethyl and cyclopentylethyl.

The term “carbocyclyl” refers to a monocyclic or multicyclic ring system of from about 3 to about 15 ring members in which all ring members are carbon atoms. Unless otherwise specified, a carbocyclyl may be saturated, partially saturated (i.e., have one or more double or triple bonds), or aromatic.

The term “heterocyclyl” refers to a monocyclic or multicyclic ring system of from about 3 to about 15 ring members in which at least one ring member is a heteroatom, such as N, O, or S. Unless otherwise specified, a heterocyclyl may be saturated, partially saturated (i.e., have one or more double or triple bonds), or aromatic. Examples of saturated and partially unsaturated non-aromatic heterocyclic groups include, but are not limited to, 3-oxetanyl, 2-oxetanyl, azetidinyl, thietanyl, tetrahydrofuranyl, tetrahydrothiophenyl, pyrrolidinyl, dihydropyranyl, tetrahydropyranyl, thio-dihydropyranyl, thio-tetrahydropyranyl, piperidinyl, piperazinyl, morpholinyl, 1,3-oxazinanyl, 1,3-thiazinanyl, 4,5,6-tetrahydropyrimidinyl, 2,3-dihydrofuranyl, dihydrothienyl, dihydropyridinyl, tetrahydropyridinyl, isoxazolidinyl, pyrazolidinyl, tetrazolyl, imidazolyl, isothiozolyl, triazolyl, azabicyclo-octanyl, diazabicyclo-octanyl, and all alkyl, alkoxy, haloalkyl and haloalkoxy substituted derivatives of any of the aforementioned groups.

The terms “cycloheteroalkyl” and “heterocycloalkyl” refer to a saturated ring system, such as a 3- to 10-member cycloalkyl ring system, that include one or more heteroatoms. The heteroatoms may be the same or different and may be nitrogen (N), oxygen (O), or sulfur (S).

Examples of heterocycloalkyl include, but are not limited to, 1-(1,2,5,6-tetrahydropyridyi), 1-piperidmyl, 2-piperidinyl, 3-piperidinyl, 4-morpholinyl, 3-morpholinyl, tetrahydrofuran-3-yl, tetrahydrofuran-3-yl, tetrahydrothien-2-yl, tetrahydrothien-3-yl, 1-piperazinyl, 2-piperazinyl, and the like.

The cycloheteroalkyl ring can be optionally fused to or otherwise attached to other cycloheteroalkyl rings and/or non-aromatic hydrocarbon rings. Heterocyclic rings include those having from one to three heteroatoms, such as oxygen, sulfur, and nitrogen, in which the nitrogen and sulfur heteroatoms may optionally be oxidized, and the nitrogen heteroatom may optionally be quaternized. Examples include, but are not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

An unsaturated hydrocarbon, carbocyclyl, or heterocyclyl has one or more double bonds or triple bonds. Examples of unsaturated hydrocarbons include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkenyl” as used herein refers to a monovalent group derived from a C₂-C₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂-C₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched, or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (CH₂)3, cyclohexylene (—C₆H₁₀—, —CH═CH—CH═CH—, —CH═CH—CH₂—, —CH₂CH₂CH₂CH₂CH₂—, —CH₂CH₂CH(CH₂CH₂CH₃)CH₂—, —(CH₂)q-N(R)—(CH₂)r-, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—).

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “spirocyclyl” refers to a polycyclic compound in which two rings have a single atom, e.g., carbon, as the only common member of two rings. Thus, a “spirocycloalkyl” refers to a cycloalkyl group with two rings having a single carbon in common, and a “spiroheterocycloalkyl” or “spiroheterocycloalkyl” refers to a cycloheteroalkyl group with two rings having a single carbon or other atom, e.g., nitrogen, in common.

The term “aryl” means, unless otherwise stated, an aromatic hydrocarbon substituent that can be a single ring or multiple rings (such as from 1 to 3 rings), which are fused together or linked covalently.

The term “heteroaryl” refers to and groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyndyl, 4-pyridyl, 2-pyrimidyl, 4-pyrimidyl, 5-benzoihiazolyl, purinyl, 2-benzimidazolyl, 5-indolyl, 1-isoquinolyl, 5-isoquinolyl, 2-quinoxalinyl, 5-quinoxalinyl, 3-qumolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

Where a heteroalkyl, heterocycloalkyl, or heteroaryl includes a specific number of members (e.g., “3 to 7 membered”), the term “member” refers to a carbon atom or heteroatom.

Each of the above terms is meant to include both substituted and unsubstituted forms of the indicated group. In some instances, the groups are explicitly defined as substituted, for example, “substituted aryl.”

When a compound includes more than one R group, for example, each of the R groups is independently selected as are each R′, R″, R″′ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of ordinary skill in the art will understand that the term “alkyl” is meant to include groups including carbon atoms bound to groups other than hydrogen groups, such as haloalkyl (e. g., —CF₃ and —CH₂CF₃) and acyl (e.g., —C(O)CH₃, —C(O)CF₃, —C(O)CH₂OCH₃, and the like).

Two of the substituents on adjacent atoms of aryl or heteroaryl ring may optionally form a ring of the formula -T-C(O)—(CRR′)_{q}-U—, wherein T and U are independently —NR—, —O—, —CRR′- or a single bond, and q is an integer from 0 to 3. Alternatively, two of the substituents on adjacent atoms of aryl or heteroaryl ring may optionally be replaced with a substituent of the formula -A-(CH2)r-B—, wherein A and B are independently —CRR′—, —O—, —NR—, —S—, —S(O)—, —S(O)₂—, —S(O)2NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)s-X′—(C″R′)d-, where s and d are independently integers of from 0 to 3, and X′ is —O—, —NR′—, —S—, —S(O)—, —S(O)₂—, or —S(O)₂NR′—. The substituents R, R′, R″ and R″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocyclic, heterocyclic, or aromatic heterocyclic group as defined herein). As such, the term “acyl” specifically includes aryl acyl groups, such as a 2-(furan-2-yl)acetyl)- and a 2-phenylacetyl group. Specific examples of acyl groups include acetyl and benzoyl. Acyl groups also are intended to include amides, —RC(═O)NR, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁-C₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxy alkyl” as used herein refers to an alkyl-O-alkyl ether, for example, a methoxy ethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described and includes substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplar)′aralkyloxyl group is benzyloxyl, i.e., C₆H₅CH₂—O—. An aralkyloxyl group can optionally be substituted.

“Alkoxycarbonyl” refers to an alkyl-O—C(═O)— group. Exemplary alkoxy carbonyl groups include methoxycarbonyl, ethoxy carbonyl, butyloxycarbonyl, and tert-butyloxycarbonyl.

“Aryloxycarbonyl” refers to an aryl-O—C(═O)— group. Exemplary aryloxy carbonyl groups include phenoxy- and naphthoxy-carbonyl.

“Aralkoxycarbonyl” refers to an aralkyl —O—C(═O)— group. An exemplary aralkoxycarbonyl group is benzyloxycarbonyl.

“Carbamoyl” refers to an amide group of the formula —C(═O)NH₂.

“Alkylcarbamoyl” refers to a R′RN —C(═O) group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term “carbonyldioxyl,” as used herein, refers to a carbonate group of the formula —OC(═O)—OR.

“Acyloxyl” refers to an acyl-O— group wherein acyl is as previously described.

The term “amino” refers to the —NH₂ group and refers to a nitrogen containing group as is known in the art derived from ammonia by the replacement of one or more hydrogen radicals by organic groups. For example, the terms “acyl amino” and “alkylamino” refer to specific N-substituted organic groups with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R″′, wherein R′, R″, and R″′ are each independently selected from the group consisting of alkyl groups. Additionally, R′, R″, and/or R″′ taken together may optionally be —(CH₂)k where k is an integer from 2 to 6. Examples include, but are not limited to, methylamino, dimethylamino, ethylamino, diethylamino, diethylaminocarbonyl, methylethylamino, isopropyl amino, piperidino, trimethylamino, and propylamine.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the COOH group. Such groups also are referred to herein as a “carboxylic acid” moiety.

The term “cyano” refers to the —CN group.

The terms “halo,” “halide,” and “halogen” refer to fluoro, chloro, bromo, and iodo groups.

The term “haloalkyl” refers to an alkyl group substituted with one or more halogens. Additionally, the term “haloalkyl,” includes monohaloalkyl and polyhaloalkyl. For example, the term “halo(C1-4)alkyl” includes, but is not limited to, trifluoromethyl, 2,2,2-trifluoroethyl, 4-chlorobutyl, 3-bromopropyl, and the like.

The terms “halocycloalky” and “cyclohaloalkyl” refer to a cycloalkly group with one or more halogens.

The term “hydroxyl” refers to the —OH group.

The term “hydroxy alkyl” refers to an alkyl group substituted with an —OH group.

The term “mercapto” refers to the —SH group.

The term “oxo” refers to an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R′.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisomeric forms that may be defined, m terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure, i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium, and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by 13C- or 14C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example, tritium (3H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts, and particularly as pharmaceutically acceptable salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates, and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or m a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow′ the compounds to be converted into either base or acid addition salts.

The neutral forms of the compounds may be regenerated by contacting the salt with a base or acid and isolating the parent compound in the conventional manner.

The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds that are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P.G.M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups.

For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

### Linkers:

The therapeutic cargo may be conjugated to the binder via a linker. The therapeutic cargo may be covalently conjugated to the binder, for example via bioconjugation.

In certain embodiments, the binder may be covalently bonded to a linker. The linker may also be connected to the therapeutic payload.

In certain embodiments, linker may be a 1-8 carbon linker. In certain embodiments, the linker is —(CH₂)ₙ—. In certain embodiments, n is 2. In certain embodiments, n is 3. In certain embodiments, n is 4. In certain embodiments, n is 3. In certain embodiments, n is 5. In certain embodiments, n is 3. In certain embodiments, n is 6. In certain embodiments, the linker is polyethylene glycol (PEG) or a derivative thereof. In certain embodiments, the linker is -(PEG)ₘ-. In certain embodiments, m is 1. In certain embodiments, m is 2. In certain embodiments, the PEG is PEG1, PEG2, PEG3, PEG4, and/or PEG8.

In certain embodiments, the linker is connected to the binder or payload by a reactive moiety. In certain preferred embodiments, the linker is connected to the binder by a reactive moiety. In certain preferred embodiments, the reactive moiety is N-hydroxy-succinimide ester (NHS). In certain preferred embodiments, the reactive moiety is azide (N₃).

### Payload Delivery Across the BBB

Disclosed herein include methods and delivery systems for delivering a payload (e.g., a therapeutic agent) to a nervous system. The method comprises providing a small molecule capable of interacting with a carbonic anhydrase IV or a derivative thereof. The small molecule can be part of a delivery system and the delivery system can comprise a payload to be delivered to a nervous system. The method can further comprise administering the delivery system to the subject.

In some embodiments, the delivery system comprises nanoparticles, nanotubes, nanowires, dendrimers, liposomes, ethosomes and aquasomes, polymersomes and niosomes, foams, hydrogels, cubosomes, quantum dots, exosomes, macrophages, and combinations thereof. In some embodiments, the delivery system comprises a nanoparticle selected from lipid-based nanoparticles, polymeric nanoparticles, inorganic nanoparticles, surfactant-based emulsions, nanowires, silica nanoparticles, virus-like particles, peptide or protein-based particles, lipid-polymer particles, nanolipoprotein particles, and combinations thereof.

For example, the payload may include an antimicrobial agent, a therapeutic agent, a prodrug, a peptide, a protein, an enzyme, a lipid, a biological response modifier, a pharmaceutical agent, a lymphokine, a heterologous antibody or fragment thereof, a detectable label, a polyethylene glycol (PEG) molecule, or a combination of two or more of the agents.

The payload can include a neuroactive polypeptide, for example, a neurotrophic factors, endocrine factors, growth factors, paracrine factors, hypothalamic release factors, neurotransmitter polypeptides, polypeptide agonists for a receptor expressed by a CNS cell, polypeptides involved in lysosomal storage disease or any combination thereof. In another example, the payload can include an IL-1 receptor antagonist (IL-1Ra), dalargin, an interferon-β, Glial-derived neurotrophic factor (GDNF), tumor necrosis factor receptor (TNFR), nerve growth factor (NGF), brain derived neurotrophic factor (BDNF), neurotrophin-4/5, neurotrophin (NT)-3, a neurturin, neuregulin, a netrin, ciliary neurotrophic factor (CNTF), stem cell factor (SCF), a semaphorin, hepatocyte growth factor (HGF), epidermal growth factor (EGF), transforming growth factor (TGF)-cx, TGF-B, vascular endothelial growth factor (VEGF), platelet-derived growth factor (PDGF), heregulin, artemin, persephin, interleukins, granulocyte-colony stimulating factor (CSF), granulocyte-macrophage-CSF, cardiotrophin-1, hedgehogs, leukemia inhibitory factor (LIF), midkine, pleiotrophin, erythropoietin (EPO), bone morphogenetic proteins (BMPs), netrins, saposins, any fragment thereof, or any combination thereof.

Aspects of the invention also provide for delivery of the conjugate to a subject in order to transport a therapeutic agent across the BBB. In aspects of the invention, delivery of the therapeutic payload may be for the treatment of a disease, disorder, or injury of the CNS. In aspects of the invention, the therapeutic agent may be released from the conjugate following entry into the CNS. In certain aspects, the disease, disorder, or injury of the CNS can be, without limitation, multiple sclerosis (MS), amylotrophic lateral sclerosis (ALS), Huntington's disease, Alzheimer's disease, Parkinson's disease, spinal cord injury, traumatic brain injury, stroke, neuropathic pain, neurodegeneration, neuroinflammation, progressive multifocal leukoencephalopathy (PML), encephalomyelitis (EPL), central pontine myelolysis (CPM), adrenoleukodystrophy, Alexander's disease, Pelizaeus Merzbacher disease (PMZ), Globoid cell Leucodystrophy (Krabbe's disease), Wallerian Degeneration, optic neuritis, transverse myelitis, post radiation injury, neurologic complications of chemotherapy, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, Bell's palsy, primary tumors, secondary metastases, or any combination thereof.

### EXPERIMENTAL EXAMPLES

### Screen of FDA-Approved Small Molecule Drugs as CA-IV Binders

A high-throughput screening was conducted using the affinity screening platform sold under the trade name NanoTemper DIANTHUS to identify effective small molecule binders from a library of approximately 1400 FDA-approved drugs. This platform measures inter-molecular interactions based on changes in fluorescence normalized ratio (Fnorm).

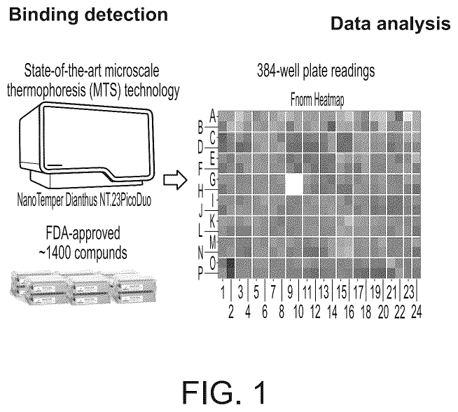

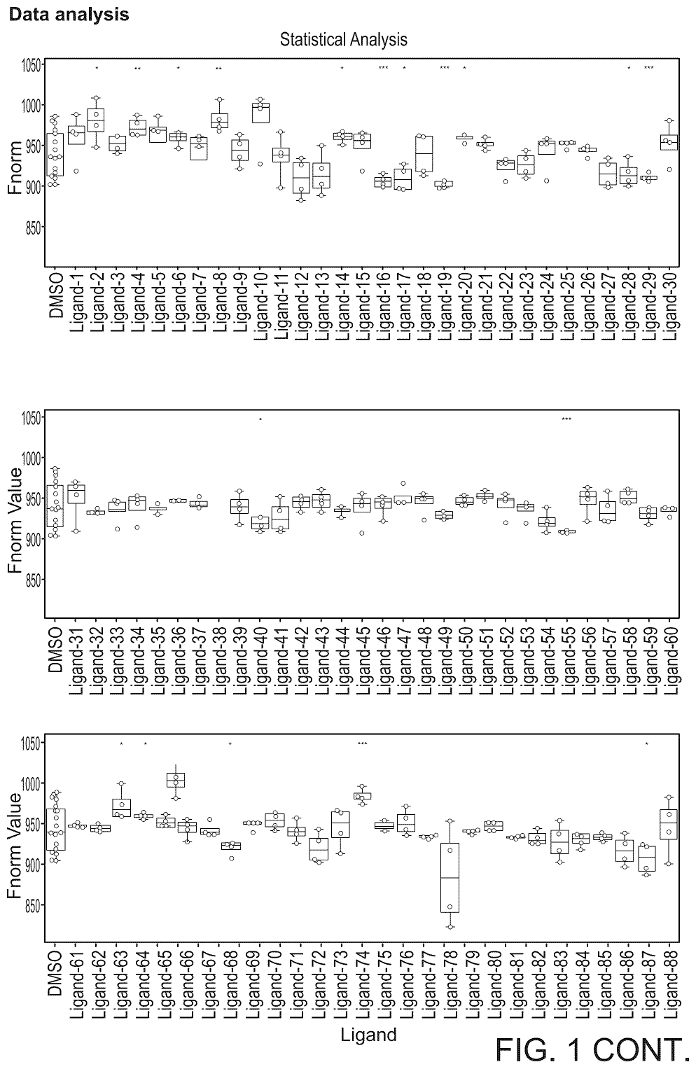

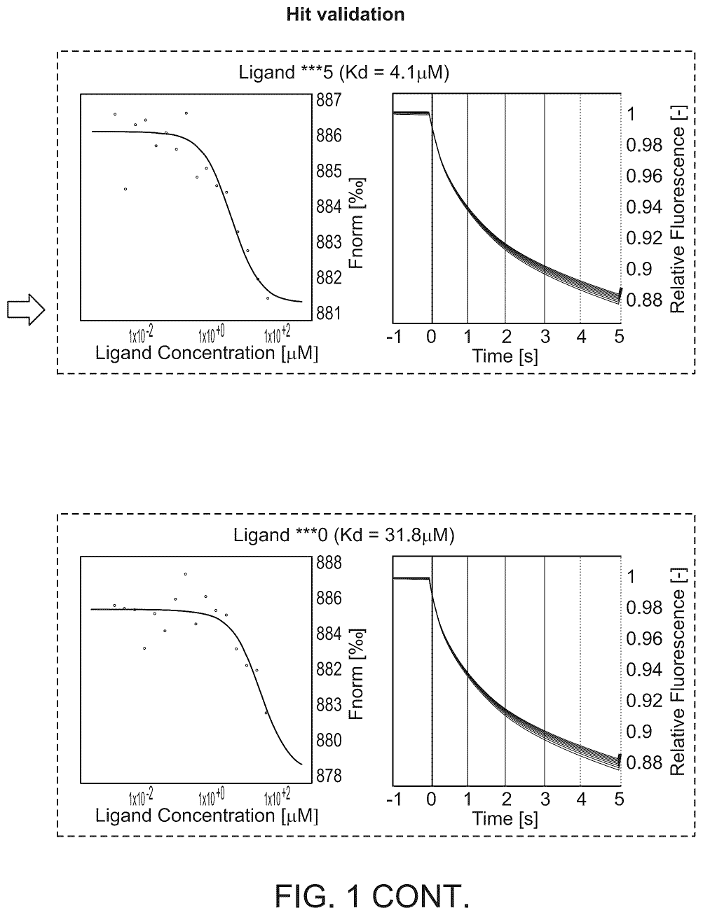

FIG. 1 is a workflow of the screening and validation process for FDA-approved drugs. Microscale thermophoresis technology was used for binding detection on 384-well plate reads.

### Method of Screening

Receptor Preparation: CA-IV was diluted to 50 nM in an assay buffer that matched with the buffer the small molecules drugs were in, composed of phosphate-buffered saline pH 7.4 with 0.5% DMSO (for drugs stored in DMSO) or phosphate-buffered saline pH7.4 alone (for drugs stored in water).

Candidate Drug Preparation: An FDA-approved drug library composed of greater than 1400 pre-dissolved compounds (Selleck, Catlog. L1300) was screened. Candidate drugs, stored as 10 mM stocks in DMSO or water, were diluted in phosphate-buffered saline, pH 7.4 to 50 μM.

Plate Preparation: Receptor proteins (10 μL/well) and drugs (10 μL/well) were loaded into 384-well plates in quadruplicates. The plates were shaken at room temperature for a duration of 2-5 hours to reach equilibrium before reading with the affinity screening platform.

Library Screening: The drug library was screened using the affinity screening platform.

Data Collection and Statistical Analysis: Fnorm readings were recorded. Hits were identified for FDA-approved drugs that showed statistical significant change in Fnorm by the screen. Hits were identified based on Student's t-test and absolute effect size comparing the drug-treated group and vehicle control group, ensuring that the identified binders caused significant change in Fnonm.

Candidates with statistical significance in the top 100 are shown in the table below:

| Rank | Plate | Ligand | Ligand name |
|---|---|---|---|
| 1 | L1300- | Ligand- | Flunarizine 2HCl |
| | 06 | 74 | |
| 2 | L1300- | Ligand- | Acetylcysteine |
| | 04 | 46 | |
| 3 | L1300- | Ligand- | Dexamethasone |
| | 02 | 40 | (DHAP) |
| 4 | L1300- | Ligand- | Ketoconazole |
| | 02 | 31 | |
| 5 | L1300- | Ligand- | Nialamide |
| | 13 | 57 | |
| 6 | L1300- | Ligand- | Ramelteon |
| | 02 | 26 | |
| 7 | L1300- | Ligand- | Drospirenone |
| | 02 | 87 | |
| 8 | L1300- | Ligand- | Escitalopram Oxalate |
| | 11 | 69 | |
| 9 | L1300- | Ligand- | Avanafil |
| | 10 | 32 | |
| 10 | L1300- | Ligand- | Ispinesib (SB- |
| | 03 | 81 | 715992) |
| 11 | L1300- | Ligand- | Trifluoperazine 2HCl |
| | 10 | 19 | |
| 12 | L1300- | Ligand- | Amphotericin B |
| | 04 | 80 | |
| 13 | L1300- | Ligand- | Cinchophen |
| | 12 | 14 | |
| 14 | L1300- | Ligand- | Lansoprazole |
| | 02 | 42 | |
| 15 | L1300- | Ligand- | Repaglinide |
| | 03 | 14 | |
| 16 | L1300- | Ligand- | Abemaciclib |
| | 15 | 81 | (LY2835219) |
| 17 | L1300- | Ligand- | Levofloxacin hydrate |
| | 14 | 76 | |
| 18 | L1300- | Ligand- | Tolperisone HCl |
| | 12 | 15 | |
| 19 | L1300- | Ligand- | Loratadine |
| | 02 | 75 | |
| 20 | L1300- | Ligand- | Mepivacaine HCl |
| | 10 | 16 | |
| 21 | L1300- | Ligand- | Zafirlukast |
| | 04 | 47 | |
| 22 | L1300- | Ligand- | Uridine |
| | 06 | 63 | |
| 23 | L1300- | Ligand- | Floxuridine |
| | 02 | 61 | |
| 24 | L1300- | Ligand- | Metaproterenol |
| | 13 | 2 | Sulfate |
| 25 | L1300- | Ligand- | Selinexor (KPT-330) |
| | 15 | 60 | |
| 26 | L1300- | Ligand- | Parecoxib |
| | 14 | 69 | |
| 27 | L1300- | Ligand- | Tiagabine |
| | 14 | 71 | |
| 28 | L1300- | Ligand- | Tivantinib (ARQ |
| | 09 | 37 | 197) |
| 29 | L1300- | Ligand- | Dextromethorphan |
| | 14 | 77 | (hydrobromide |
| | | | hydrate) |
| 30 | L1300- | Ligand- | Noscapine HCl |
| | 13 | 4 | |
| 31 | L1300- | Ligand- | Moxalactam |
| | 13 | 35 | Disodium |
| 32 | L1300- | Ligand- | Sulfapyridine |
| | 04 | 67 | |
| 33 | L1300- | Ligand- | Daptomycin |
| | 16 | 40 | |
| 34 | L1300- | Ligand- | Barasertib |
| | 01 | 62 | (AZD1152-HQPA) |
| 35 | L1300- | Ligand- | Dovitinib (TKI-258, |
| | 01 | 46 | CHIR-258) |
| 36 | L1300- | Ligand- | Silodosin |
| | 04 | 34 | |
| 37 | L1300- | Ligand- | Sulfameter |
| | 04 | 78 | |
| 38 | L1300- | Ligand- | Amorolfine HCl |
| | 04 | 29 | |
| 39 | L1300- | Ligand- | Carbidopa |
| | 05 | 55 | |
| 40 | L1300- | Ligand- | Zinc Pyrithione |
| | 11 | 70 | |
| 41 | L1300- | Ligand- | Ciclesonide |
| | 14 | 68 | |
| 42 | L1300- | Ligand- | Sodium 4- |
| | 13 | 72 | aminohippurate |
| | | | Hydrate |
| 43 | L1300- | Ligand- | Proadifen HCl |
| | 13 | 16 | |
| 44 | L1300- | Ligand- | Diiodohydroxyquinoline |
| | 14 | 25 | |
| 45 | L1300- | Ligand- | Doripenem Hydrate |
| | 02 | 65 | |
| 46 | L1300- | Ligand- | Mepiroxol |
| | 13 | 67 | |
| 47 | L1300- | Ligand- | Docetaxel |
| | 01 | 73 | |
| 48 | L1300- | Ligand- | Esomeprazole |
| | 05 | 46 | Magnesium |
| 49 | L1300- | Ligand- | Rivaroxaban |
| | 09 | 51 | |
| 50 | L1300- | Ligand- | Cyclizine 2HCl |
| | 11 | 86 | |
| 51 | L1300- | Ligand- | Nitazoxanide |
| | 04 | 3 | |
| 52 | L1300- | Ligand- | Acetaminophen |
| | 04 | 58 | |
| 53 | L1300- | Ligand- | Aceclidine HCl |
| | 13 | 81 | |
| 54 | L1300- | Ligand- | Benzocaine |
| | 12 | 16 | |
| 55 | L1300- | Ligand- | Erythromycin |
| | 04 | 69 | |
| 56 | L1300- | Ligand- | Milnacipran HCl |
| | 10 | 26 | |
| 57 | L1300- | Ligand- | Gliquidone |
| | 10 | 81 | |
| 58 | L1300- | Ligand- | Sertaconazole nitrate |
| | 10 | 38 | |
| 59 | L1300- | Ligand- | Prednisone |
| | 04 | 35 | |
| 60 | L1300- | Ligand- | Clomifene citrate |
| | 08 | 53 | |
| 61 | L1300- | Ligand- | Fedratinib; |
| | 09 | 26 | TG101348 |
| | | | (SAR302503) |
| 62 | L1300- | Ligand- | Resorcinol |
| | 14 | 38 | |
| 63 | L1300- | Ligand- | Escin |
| | 15 | 24 | |
| 64 | L1300- | Ligand- | Polymyxin B |
| | 16 | 73 | sulphate |
| 65 | L1300- | Ligand- | Sunitinib Malate |
| | 01 | 4 | |
| 66 | L1300- | Ligand- | Oxeladin Citrate |
| | 13 | 39 | |
| 67 | L1300- | Ligand- | Fexofenadine HCl |
| | 10 | 41 | |
| 68 | L1300- | Ligand- | Pantoprazole sodium |
| | 13 | 86 | |
| 69 | L1300- | Ligand- | Aminocaproic acid |
| | 04 | 83 | |
| 70 | L1300- | Ligand- | Prilocaine |
| | 04 | 2 | |
| 71 | L1300- | Ligand- | Metolazone |
| | 04 | 12 | |
| 72 | L1300- | Ligand- | Ribociclib (LEE011) |
| | 15 | 18 | |
| 73 | L1300- | Ligand- | Telbivudine |
| | 04 | 38 | |
| 74 | L1300- | Ligand- | Florfenicol |
| | 12 | 26 | |
| 75 | L1300- | Ligand- | Trimethoprim |
| | 10 | 36 | |
| 76 | L1300- | Ligand- | Allopurinol |
| | 04 | 36 | |
| 77 | L1300- | Ligand- | Isoetharine Mesylate |
| | 13 | 56 | |
| 78 | L1300- | Ligand- | Procyclidine HCl |
| | 13 | 3 | |
| 79 | L1300- | Ligand- | Nifenazone |
| | 13 | 28 | |
| 80 | L1300- | Ligand- | Aminophylline |
| | 04 | 18 | |
| 81 | L1300- | Ligand- | Sodium Nitrite |
| | 11 | 59 | |
| 82 | L1300- | Ligand- | Omipalisib; |
| | 09 | 14 | GSK2126458 |
| | | | (GSK458) |
| 83 | L1300- | Ligand- | Rizatriptan Benzoate |
| | 03 | 77 | |
| 84 | L1300- | Ligand- | Riluzole |
| | 04 | 45 | |
| 85 | L1300- | Ligand- | Ropinirole HCl |
| | 10 | 51 | |
| 86 | L1300- | Ligand- | Articaine HCl |
| | 10 | 70 | |
| 87 | L1300- | Ligand- | Tylosin tartrate |
| | 10 | 49 | |
| 88 | L1300- | Ligand- | Mezlocillin Sodium |
| | 12 | 24 | |
| 89 | L1300- | Ligand- | Theophylline |
| | 04 | 24 | |
| 90 | L1300- | Ligand- | Oxybenzone |
| | 15 | 35 | |
| 91 | L1300- | Ligand- | Cinepazide maleate |
| | 09 | 10 | |
| 92 | L1300- | Ligand- | Ipratropium Bromide |
| | 04 | 84 | |
| 93 | L1300- | Ligand- | Sildenafil Mesylate |
| | 15 | 45 | |
| 94 | L1300- | Ligand- | Ceritinib (LDK378) |
| | 15 | 26 | |
| 95 | L1300- | Ligand- | Risperidone |
| | 04 | 56 | |
| 96 | L1300- | Ligand- | Benztropine mesylate |
| | 10 | 60 | |
| 97 | L1300- | Ligand- | Aceglutamide |
| | 13 | 73 | |
| 98 | L1300- | Ligand- | Alendronate |
| | 04 | 57 | |
| 99 | L1300- | Ligand- | Fenofibric acid |
| | 13 | 9 | |
| 100 | L1300- | Ligand- | Entrectinib (RXDX- |
| | 15 | 55 | 101) |

The complete list of the screened compounds is provided in the table below:

| Plate | Ligand | Ligand name |
|---|---|---|
| L1300-01 | Ligand-62 | Barasertib (AZD1152-HQPA) |
| L1300-01 | Ligand-46 | Dovitinib (TKI-258, CHIR-258) |
| L1300-01 | Ligand-73 | Docetaxel |
| L1300-01 | Ligand-4 | Sunitinib Malate |
| L1300-01 | Ligand-35 | Cediranib (AZD2171) |
| L1300-01 | Ligand-18 | Alisertib (MLN8237) |
| L1300-01 | Ligand-45 | Saracatinib (AZD0530) |
| L1300-01 | Ligand-49 | Belinostat (PXD101) |
| L1300-01 | Ligand-5 | Masitinib (AB1010) |
| L1300-01 | Ligand-8 | Roscovitine (Seliciclib, CYC202) |
| L1300-01 | Ligand-39 | Foretinib (GSK1363089) |
| L1300-01 | Ligand-78 | Nintedanib (BIBF1120) |
| L1300-01 | Ligand-41 | Ganetespib (STA-9090) |
| L1300-01 | Ligand-27 | Crizotinib (PF-02341066) |
| L1300-01 | Ligand-47 | Nilotinib (AMN-107) |
| L1300-01 | Ligand-14 | Lapatinib (GW-572016) Ditosylate |
| L1300-01 | Ligand-7 | YM155 (Sepantronium Bromide) |
| L1300-01 | Ligand-24 | Bosutinib (SKI-606) |
| L1300-01 | Ligand-82 | OSI-906 (Linsitinib) |
| L1300-01 | Ligand-29 | Malotilate |
| L1300-01 | Ligand-34 | Axitinib |
| L1300-01 | Ligand-70 | Enzastaurin (LY317615) |
| L1300-01 | Ligand-61 | Cabozantinib (XL184, BMS-907351) |
| L1300-01 | Ligand-64 | Raltitrexed |
| L1300-01 | Ligand-50 | Triciribine |
| L1300-01 | Ligand-69 | Rapamycin (Sirolimus) |
| L1300-01 | Ligand-56 | FG-4592 |
| L1300-01 | Ligand-51 | Ivacaftor (VX-770) |
| L1300-01 | Ligand-31 | Aprepitant |
| L1300-01 | Ligand-48 | VX-680 (Tozasertib, MK-0457) |
| L1300-01 | Ligand-1 | ABT-263 (Navitoclax) |
| L1300-01 | Ligand-40 | 17-AAG (Tanespimycin) |
| L1300-01 | Ligand-63 | Lenvatinib (E7080) |
| L1300-01 | Ligand-59 | Entinostat (MS-275) |
| L1300-01 | Ligand-6 | Quisinostat (JNJ-26481585) |
| L1300-01 | Ligand-15 | Temsirolimus (CCI-779, NSC683864) |
| L1300-01 | Ligand-16 | Pictilisib (GDC-0941) |
| L1300-01 | Ligand-86 | CUDC-101 |
| L1300-01 | Ligand-84 | Paclitaxel |
| L1300-01 | Ligand-57 | Dasatinib |
| L1300-01 | Ligand-60 | Iniparib (BSI-201) |
| L1300-01 | Ligand-76 | Dimesna |
| L1300-01 | Ligand-83 | Mocetinostat (MGCD0103) |
| L1300-01 | Ligand-28 | Vatalanib (PTK787) 2HCl |
| L1300-01 | Ligand-38 | Vismodegib (GDC-0449) |
| L1300-01 | Ligand-71 | PCI-24781 (Abexinostat) |
| L1300-01 | Ligand-3 | Imatinib Mesylate (STI571) |
| L1300-01 | Ligand-9 | Ritonavir |
| L1300-01 | Ligand-53 | Fulvestrant |
| L1300-01 | Ligand-66 | Methotrexate |
| L1300-01 | Ligand-25 | Lenalidomide (CC-5013) |
| L1300-01 | Ligand-23 | Veliparib (ABT-888) |
| L1300-01 | Ligand-20 | Anastrozole |
| L1300-01 | Ligand-77 | Imiquimod |
| L1300-01 | Ligand-44 | Doxorubicin (Adriamycin) |
| L1300-01 | Ligand-87 | Dutasteride |
| L1300-01 | Ligand-88 | Bendamustine HCl |
| L1300-01 | Ligand-11 | Melatonin |
| L1300-01 | Ligand-2 | Afatinib (BIBW2992) |
| L1300-01 | Ligand-32 | Finasteride |
| L1300-01 | Ligand-10 | TAK-700 (Orteronel) |
| L1300-01 | Ligand-81 | Olaparib (AZD2281, Ku-0059436) |
| L1300-01 | Ligand-75 | Thalidomide |
| L1300-01 | Ligand-85 | Regorafenib (BAY 73-4506) |
| L1300-01 | Ligand-67 | Selumetinib (AZD6244) |
| L1300-01 | Ligand-12 | Linifanib (ABT-869) |
| L1300-01 | Ligand-37 | Vorinostat (SAHA, MK0683) |
| L1300-01 | Ligand-52 | Onalespib (AT13387) |
| L1300-01 | Ligand-13 | Bortezomib (PS-341) |
| L1300-01 | Ligand-72 | Everolimus (RAD001) |
| L1300-01 | Ligand-65 | Decitabine |
| L1300-01 | Ligand-36 | Panobinostat (LBH589) |
| L1300-01 | Ligand-80 | Sorafenib Tosylate |
| L1300-01 | Ligand-54 | Cladribine |
| L1300-01 | Ligand-55 | Fluorouracil (5-Fluoracil, 5-FU) |
| L1300-01 | Ligand-42 | Bicalutamide |
| L1300-01 | Ligand-21 | Exemestane |
| L1300-01 | Ligand-33 | Tivozanib (AV-951) |
| L1300-01 | Ligand-19 | Capecitabine |
| L1300-01 | Ligand-68 | Erlotinib HCl (OSI-744) |
| L1300-01 | Ligand-58 | Pazopanib HCl |
| L1300-01 | Ligand-79 | Gefitinib (ZD1839) |
| L1300-01 | Ligand-74 | Valproic acid sodium salt (Sodium |
| | | valproate) |
| L1300-01 | Ligand-17 | Rucaparib (AG-014699, PF-01367338) |
| L1300-01 | Ligand-30 | CEP-18770 (Delanzomib) |
| L1300-01 | Ligand-43 | Irinotecan |
| L1300-01 | Ligand-26 | Vandetanib (ZD6474) |
| L1300-01 | Ligand-22 | Bisoprolol fumarate |
| L1300-02 | Ligand-40 | Dexamethasone (DHAP) |
| L1300-02 | Ligand-31 | Ketoconazole |
| L1300-02 | Ligand-26 | Ramelteon |
| L1300-02 | Ligand-87 | Drospirenone |
| L1300-02 | Ligand-42 | Lansoprazole |
| L1300-02 | Ligand-75 | Loratadine |
| L1300-02 | Ligand-61 | Floxuridine |
| L130002 | Ligand-65 | Doripenem Hydrate |
| L1300-02 | Ligand-88 | Omeprazole |
| L1300-02 | Ligand-50 | Cilostazol |
| L1300-02 | Ligand-69 | Nepafenac |
| L1300-02 | Ligand-80 | Rufinamide |
| L1300-02 | Ligand-14 | Amuvatinib (MP-470) |
| L1300-02 | Ligand-48 | Celecoxib |
| L1300-02 | Ligand-27 | Amisulpride |
| L1300-02 | Ligand-20 | Ivermectin |
| L1300-02 | Ligand-72 | Tegafur (FT-207) (NSC148958) |
| L1300-02 | Ligand-56 | Epirubicin HCl |
| L1300-02 | Ligand-9 | Glimepiride |
| L1300-02 | Ligand-30 | Fluconazole |
| L1300-02 | Ligand-13 | Fludarabine Phosphate |
| L1300-02 | Ligand-76 | Gestodene |
| L1300-02 | Ligand-16 | Altretamine |
| L1300-02 | Ligand-19 | Felbamate |
| L1300-02 | Ligand-64 | Lidocaine |
| L1300-02 | Ligand-71 | Benazepril HCl |
| L1300-02 | Ligand-29 | Streptozotocin (STZ) |
| L1300-02 | Ligand-32 | Amonafide |
| L1300-02 | Ligand-79 | Vincristine |
| L1300-02 | Ligand-70 | Vemurafenib (PLX4032, RG7204) |
| L1300-02 | Ligand-25 | Leflunomide |
| L1300-02 | Ligand-3 | Agomelatine |
| L1300-02 | Ligand-24 | Flavopiridol (Alvocidib) |
| L1300-02 | Ligand-11 | Ruxolitinib (INCB018424) |
| L1300-02 | Ligand-18 | Mercaptopurine (6-MP) |
| L1300-02 | Ligand-57 | Temozolomide |
| L1300-02 | Ligand-85 | Genistein |
| L1300-02 | Ligand-39 | Cilnidipine |
| L1300-02 | Ligand-74 | Gatifloxacin |
| L1300-02 | Ligand-68 | Tamoxifen |
| L1300-02 | Ligand-23 | Clofarabine |
| L1300-02 | Ligand-49 | Artemisinin |
| L1300-02 | Ligand-41 | Flumazenil |
| L1300-02 | Ligand-58 | Entecavir Hydrate |
| L1300-02 | Ligand-28 | Ceterizine DiHCl |
| L1300-02 | Ligand-8 | Etomidate |
| L1300-02 | Ligand-60 | Asenapine |
| L1300-02 | Ligand-46 | Letrozole |
| L1300-02 | Ligand-22 | Isotretinoin |
| L1300-02 | Ligand-81 | Acarbose |
| L1300-02 | Ligand-66 | Mosapride Citrate |
| L1300-02 | Ligand-47 | Dienogest |
| L1300-02 | Ligand-17 | Carmofur |
| L1300-02 | Ligand-38 | Aniracetam |
| L1300-02 | Ligand-77 | Nafamostat Mesylate |
| L1300-02 | Ligand-44 | Meropenem |
| L1300-02 | Ligand-73 | Ellagic acid |
| L1300-02 | Ligand-86 | Losartan Potassium (DuP 753) |
| L1300-02 | Ligand-53 | Levetiracetam |
| L1300-02 | Ligand-1 | Nelarabine |
| L1300-02 | Ligand-12 | Bleomycin Sulfate |
| L1300-02 | Ligand-10 | Rigosertib (ON-01910) |
| L1300-02 | Ligand-43 | Acetretin |
| L1300-02 | Ligand-7 | Megestrol Acetate |
| L1300-02 | Ligand-67 | Oxaliplatin |
| L1300-02 | Ligand-4 | Posaconazole |
| L1300-02 | Ligand-15 | Prasugrel |
| L1300-02 | Ligand-33 | Lopinavir |
| L1300-02 | Ligand-78 | Etoposide |
| L1300-02 | Ligand-62 | Edaravone |
| L1300-02 | Ligand-34 | Dacarbazine |
| L1300-02 | Ligand-21 | Epothilone B (EPO906, Patupilone) |
| L1300-02 | Ligand-55 | Mianserin HCl |
| L1300-02 | Ligand-59 | Avagacestat (BMS-708163) |
| L1300-02 | Ligand-51 | Doxazosin Mesylate |
| L1300-02 | Ligand-54 | Bafetinib (INNO-406) |
| L1300-02 | Ligand-82 | Budesonide |
| L1300-02 | Ligand-2 | Raloxifene HCl |
| L1300-02 | Ligand-5 | Adapalene |
| L1300-02 | Ligand-45 | Dexrazoxane HCl (ICRF-187, ADR-529) |
| L1300-02 | Ligand-37 | Cinacalcet HCl |
| L1300-02 | Ligand-35 | Topotecan HCl |
| L1300-02 | Ligand-52 | Fluoxetine HCl |
| L1300-02 | Ligand-36 | Enzalutamide (MDV3100) |
| L1300-02 | Ligand-6 | Bumetanide |
| L1300-02 | Ligand-84 | Etodolac |
| L1300-02 | Ligand-83 | Ifosfamide |
| L1300-02 | Ligand-63 | Fluvoxamine maleate |
| L1300-03 | Ligand-81 | Ispinesib (SB-715992) |
| L1300-03 | Ligand-14 | Repaglinide |
| L1300-03 | Ligand-77 | Rizatriptan Benzoate |
| L1300-03 | Ligand-86 | Pimobendan |
| L1300-03 | Ligand-39 | VX-222 (VCH-222, Lomibuvir) |
| L1300-03 | Ligand-40 | Betamethasone |
| L1300-03 | Ligand-7 | Ponatinib (AP24534) |
| L1300-03 | Ligand-58 | Tamsulosin |
| L1300-03 | Ligand-87 | Apixaban |
| L1300-03 | Ligand-9 | Quizartinib (AC220) |
| L1300-03 | Ligand-50 | Zosuquidar (LY335979) 3HCl |
| L1300-03 | Ligand-65 | Sulfasalazine |
| L1300-03 | Ligand-69 | Tianeptine sodium |
| L1300-03 | Ligand-13 | Tigecycline |
| L1300-03 | Ligand-59 | Ziprasidone HCl |
| L1300-03 | Ligand-88 | Pyridostigmine Bromide |
| L1300-03 | Ligand-79 | Prazosin HCl |
| L1300-03 | Ligand-85 | SAR245409 (XL765) |
| L1300-03 | Ligand-60 | Moxifloxacin HCl |
| L1300-03 | Ligand-78 | Tenofovir Disoproxil Fumarate |
| L1300-03 | Ligand-73 | Aztreonam |
| L1300-03 | Ligand-5 | Cilomilast |
| L1300-03 | Ligand-51 | Mycophenolate Mofetil |
| L1300-03 | Ligand-41 | Tadalafil |
| L1300-03 | Ligand-62 | Dyphylline |
| L1300-03 | Ligand-76 | Candesartan |
| L1300-03 | Ligand-52 | Cyclosporine |
| L1300-03 | Ligand-32 | Tazarotene |
| L1300-03 | Ligand-70 | Zonisamide |
| L1300-03 | Ligand-80 | Tizanidine HCl |
| L1300-03 | Ligand-45 | Resveratrol |
| L1300-03 | Ligand-48 | Zileuton |
| L1300-03 | Ligand-20 | Telaprevir (VX-950) |
| L1300-03 | Ligand-46 | Linezolid |
| L1300-03 | Ligand-56 | Rocuronium Bromide |
| L1300-03 | Ligand-36 | Sildenafil Citrate |
| L1300-03 | Ligand-16 | Zibotentan (ZD4054) |
| L1300-03 | Ligand-26 | Venlafaxine |
| L1300-03 | Ligand-57 | Alfuzosin HCl |
| L1300-03 | Ligand-74 | Natamycin |
| L1300-03 | Ligand-68 | Clopidogrel |
| L1300-03 | Ligand-72 | Iloperidone |
| L1300-03 | Ligand-67 | Stavudine (d4T) |
| L1300-03 | Ligand-2 | Tenofovir |
| L1300-03 | Ligand-10 | VX-809 (Lumacaftor) |
| L1300-03 | Ligand-75 | Nebivolol |
| L1300-03 | Ligand-47 | Sumatriptan Succinate |
| L1300-03 | Ligand-53 | Febuxostat |
| L1300-03 | Ligand-42 | EX 527 (Selisistat) |
| L1300-03 | Ligand-49 | Marbofloxacin |
| L1300-03 | Ligand-19 | Norfloxacin |
| L1300-03 | Ligand-22 | Reserpine |
| L1300-03 | Ligand-33 | Furosemide |
| L1300-03 | Ligand-27 | Atazanavir Sulfate |
| L1300-03 | Ligand-61 | Daclatasvir (BMS-790052) |
| L1300-03 | Ligand-82 | Alfacalcidol |
| L1300-03 | Ligand-63 | Pracinostat (SB939) |
| L1300-03 | Ligand-64 | Dapagliflozin |
| L1300-03 | Ligand-66 | Clotrimazole |
| L1300-03 | Ligand-24 | Trilostane |
| L1300-03 | Ligand-43 | Fasudil (HA-1077) HCl |
| L1300-03 | Ligand-35 | Vecuronium Bromide |
| L1300-03 | Ligand-18 | Fludarabine |
| L1300-03 | Ligand-8 | Alprostadil |
| L1300-03 | Ligand-30 | Lactulose |
| L1300-03 | Ligand-4 | Topiramate |
| L1300-03 | Ligand-28 | Pimasertib (AS-703026) |
| L1300-03 | Ligand-83 | Naratriptan |
| L1300-03 | Ligand-31 | Saxagliptin |
| L1300-03 | Ligand-54 | BIRB 796 (Doramapimod) |
| L1300-03 | Ligand-1 | Ondansetron HCl |
| L1300-03 | Ligand-25 | Rolipram |
| L1300-03 | Ligand-71 | Doxercalciferol |
| L1300-03 | Ligand-23 | Pelitinib (EKB-569) |
| L1300-03 | Ligand-6 | TSU-68 (SU6668, Orantinib) |
| L1300-03 | Ligand-44 | Olmesartan Medoxomil |
| L1300-03 | Ligand-37 | Voriconazole |
| L1300-03 | Ligand-55 | Cefdinir |
| L1300-03 | Ligand-38 | Ofloxacin |
| L1300-03 | Ligand-84 | Irbesartan |
| L1300-03 | Ligand-12 | Oxcarbazepine |
| L1300-03 | Ligand-29 | Pralatrexate |
| L1300-03 | Ligand-21 | Pomalidomide |
| L1300-03 | Ligand-34 | Pizotifen Malate |
| L1300-03 | Ligand-15 | Tranilast |
| L1300-03 | Ligand-3 | Ranolazine 2HCl |
| L1300-03 | Ligand-17 | Safinamide Mesylate |
| L1300-03 | Ligand-11 | Semagacestat (LY450139) |
| L1300-04 | Ligand-46 | Acetylcysteine |
| L1300-04 | Ligand-80 | Amphotericin B |
| L1300-04 | Ligand-47 | Zafirlukast |
| L1300-04 | Ligand-67 | Sulfapyridine |
| L1300-04 | Ligand-34 | Silodosin |
| L1300-04 | Ligand-78 | Sulfameter |
| L1300-04 | Ligand-29 | Amorolfine HCl |
| L1300-04 | Ligand-3 | Nitazoxanide |
| L1300-04 | Ligand-58 | Acetaminophen |
| L1300-04 | Ligand-69 | Erythromycin |
| L1300-04 | Ligand-35 | Prednisone |
| L1300-04 | Ligand-83 | Aminocaproic acid |
| L1300-04 | Ligand-2 | Prilocaine |
| L1300-04 | Ligand-12 | Metolazone |
| L1300-04 | Ligand-38 | Telbivudine |
| L1300-04 | Ligand-36 | Allopurinol |
| L1300-04 | Ligand-18 | Aminophylline |
| L1300-04 | Ligand-45 | Riluzole |
| L1300-04 | Ligand-24 | Theophylline |
| L1300-04 | Ligand-84 | Ipratropium Bromide |
| L1300-04 | Ligand-56 | Risperidone |
| L1300-04 | Ligand-57 | Alendronate |
| L1300-04 | Ligand-5 | Ketorolac |
| L1300-04 | Ligand-61 | Trichlormethiazide |
| L1300-04 | Ligand-4 | Ibuprofen |
| L1300-04 | Ligand-10 | Hydrochlorothiazide |
| L1300-04 | Ligand-11 | Adefovir Dipivoxil |
| L1300-04 | Ligand-9 | Torsemide |
| L1300-04 | Ligand-70 | Nitrofural |
| L1300-04 | Ligand-68 | Ethinyl Estradiol |
| L1300-04 | Ligand-17 | Dofetilide |
| L1300-04 | Ligand-23 | Cefoperazone |
| L1300-04 | Ligand-16 | Adenosine |
| L1300-04 | Ligand-72 | Loteprednol etabonate |
| L1300-04 | Ligand-13 | Darunavir Ethanolate |
| L1300-04 | Ligand-71 | Phenylbutazone |
| L1300-04 | Ligand-1 | Methimazole |
| L1300-04 | Ligand-59 | Ursodiol |
| L1300-04 | Ligand-50 | Flucytosine |
| L1300-04 | Ligand-27 | Zolmitriptan |
| L1300-04 | Ligand-85 | Hydrocortisone |
| L1300-04 | Ligand-6 | Enalaprilat Dihydrate |
| L1300-04 | Ligand-79 | Naproxen |
| L1300-04 | Ligand-8 | Sulfanilamide |
| L1300-04 | Ligand-20 | Desonide |
| L1300-04 | Ligand-81 | Ketoprofen |
| L1300-04 | Ligand-14 | Triamcinolone Acetonide |
| L1300-04 | Ligand-21 | Estradiol |
| L1300-04 | Ligand-75 | Lamivudine |
| L1300-04 | Ligand-49 | Monobenzone |
| L1300-04 | Ligand-48 | Methyldopa |
| L1300-04 | Ligand-37 | Chlorothiazide |
| L1300-04 | Ligand-7 | Aminoglutethimide |
| L1300-04 | Ligand-15 | Amprenavir |
| L1300-04 | Ligand-62 | Disulfiram |
| L1300-04 | Ligand-28 | Isradipine |
| L1300-04 | Ligand-51 | Flurbiprofen |
| L1300-04 | Ligand-87 | Fomepizole |
| L1300-04 | Ligand-26 | Albendazole |
| L1300-04 | Ligand-74 | Carbamazepine |
| L1300-04 | Ligand-30 | Meprednisone |
| L1300-04 | Ligand-66 | Terbinafine |
| L1300-04 | Ligand-86 | Eplerenone |
| L1300-04 | Ligand-40 | Chloramphenicol |
| L1300-04 | Ligand-22 | Zalcitabine |
| L1300-04 | Ligand-43 | Piroxicam |
| L1300-04 | Ligand-33 | Azathioprine |
| L1300-04 | Ligand-44 | Indomethacin |
| L1300-04 | Ligand-31 | Didanosine |
| L1300-04 | Ligand-25 | Orlistat |
| L1300-04 | Ligand-63 | Busulfan |
| L1300-04 | Ligand-52 | Praziquantel |
| L1300-04 | Ligand-54 | Gemcitabine |
| L1300-04 | Ligand-41 | Betamethasone valerate (Betnovate) |
| L1300-04 | Ligand-32 | Deferasirox |
| L1300-04 | Ligand-73 | Mesalamine |
| L1300-04 | Ligand-42 | Divalproex Sodium |
| L1300-04 | Ligand-53 | Emtricitabine |
| L1300-04 | Ligand-60 | Tretinoin |
| L1300-04 | Ligand-77 | Levonorgestrel |
| L1300-04 | Ligand-55 | Paliperidone |
| L1300-04 | Ligand-76 | Glyburide |
| L1300-04 | Ligand-65 | Glipizide |
| L1300-04 | Ligand-39 | Estrone |
| L1300-04 | Ligand-19 | Betamethasone Dipropionate |
| L1300-04 | Ligand-64 | Progesterone |
| L1300-04 | Ligand-82 | Ezetimibe |
| L1300-04 | Ligand-88 | Gemfibrozil |
| L1300-05 | Ligand-55 | Carbidopa |
| L1300-05 | Ligand-46 | Esomeprazole Magnesium |
| L1300-05 | Ligand-60 | Simvastatin |
| L1300-05 | Ligand-30 | Butoconazole nitrate |
| L1300-05 | Ligand-84 | Oxfendazole |
| L1300-05 | Ligand-83 | Amiloride HCl |
| L1300-05 | Ligand-16 | Teniposide |
| L1300-05 | Ligand-9 | Chenodeoxycholic Acid |
| L1300-05 | Ligand-62 | Proparacaine HCl |
| L1300-05 | Ligand-42 | Curcumin |
| L1300-05 | Ligand-64 | Pefloxacin Mesylate |
| L1300-05 | Ligand-34 | Meloxicam |
| L1300-05 | Ligand-11 | Sparfloxacin |
| L1300-05 | Ligand-76 | Protionamide |
| L1300-05 | Ligand-24 | Rifabutin |
| L1300-05 | Ligand-8 | Carvedilol |
| L1300-05 | Ligand-49 | Rifaximin |
| L1300-05 | Ligand-75 | Metoprolol Tartrate |
| L1300-05 | Ligand-45 | Mesna |
| L1300-05 | Ligand-73 | Pranlukast |
| L1300-05 | Ligand-80 | Rifampin |
| L1300-05 | Ligand-87 | Idoxuridine |
| L1300-05 | Ligand-10 | Diltiazem HCl |
| L1300-05 | Ligand-85 | Lomustine |
| L1300-05 | Ligand-77 | Dipyridamole |
| L1300-05 | Ligand-43 | Valaciclovir HCl |
| L1300-05 | Ligand-81 | Azacitidine |
| L1300-05 | Ligand-15 | Sulfadiazine |
| L1300-05 | Ligand-7 | Amlodipine besylate (Norvasc) |
| L1300-05 | Ligand-35 | Nevirapine |
| L1300-05 | Ligand-54 | Ganciclovir |
| L1300-05 | Ligand-48 | Toremifene Citrate |
| L1300-05 | Ligand-33 | Deflazacort |
| L1300-05 | Ligand-88 | Hydroxyurea |
| L1300-05 | Ligand-78 | Telmisartan |
| L1300-05 | Ligand-29 | Fenoprofen Calcium |
| L1300-05 | Ligand-69 | Quetiapine Fumarate |
| L1300-05 | Ligand-1 | Indapamide |
| L1300-05 | Ligand-40 | Erdosteine |
| L1300-05 | Ligand-82 | Fenofibrate |
| L1300-05 | Ligand-61 | Aciclovir |
| L1300-05 | Ligand-22 | Felodipine |
| L1300-05 | Ligand-31 | Clemastine Fumarate |
| L1300-05 | Ligand-71 | Ramipril |
| L1300-05 | Ligand-25 | Pitavastatin Calcium |
| L1300-05 | Ligand-63 | Flubendazole |
| L1300-05 | Ligand-27 | Stanozolol |
| L1300-05 | Ligand-51 | Betaxolol hydrochloride (Betoptic) |
| L1300-05 | Ligand-65 | Roxatidine Acetate HCl |
| L1300-05 | Ligand-53 | Bifonazole |
| L1300-05 | Ligand-72 | Nifedipine |
| L1300-05 | Ligand-50 | Acipimox |
| L1300-05 | Ligand-20 | Cimetidine |
| L1300-05 | Ligand-28 | Acadesine |
| L1300-05 | Ligand-21 | Diphenhydramine HCl |
| L1300-05 | Ligand-70 | Trifluridine |
| L1300-05 | Ligand-47 | Suprofen |
| L1300-05 | Ligand-36 | Rifapentine |
| L1300-05 | Ligand-68 | Nimodipine |
| L1300-05 | Ligand-56 | Methocarbamol |
| L1300-05 | Ligand-4 | Cefditoren Pivoxil |
| L1300-05 | Ligand-79 | Nisoldipine |
| L1300-05 | Ligand-67 | Prednisolone |
| L1300-05 | Ligand-19 | Atracurium Besylate |
| L1300-05 | Ligand-2 | Thiabendazole |
| L1300-05 | Ligand-3 | Oxybutynin |
| L1300-05 | Ligand-41 | Azithromycin |
| L1300-05 | Ligand-12 | Mitotane |
| L1300-05 | Ligand-14 | Enoxacin |
| L1300-05 | Ligand-52 | Albendazole Oxide |
| L1300-05 | Ligand-44 | Nizatidine |
| L1300-05 | Ligand-58 | Pyrazinamide |
| L1300-05 | Ligand-18 | Chlorpheniramine Maleate |
| L1300-05 | Ligand-17 | Ranitidine |
| L1300-05 | Ligand-74 | Chloroxine |
| L1300-05 | Ligand-5 | Vidarabine |
| L1300-05 | Ligand-37 | Oxytetracycline (Terramycin) |
| L1300-05 | Ligand-6 | Ranolazine |
| L1300-05 | Ligand-13 | Guaifenesin |
| L1300-05 | Ligand-26 | Chlorprothixene |
| L1300-05 | Ligand-32 | Dapoxetine HCl |
| L1300-05 | Ligand-39 | Acetylcholine Chloride |
| L1300-05 | Ligand-59 | Ethionamide |
| L1300-05 | Ligand-86 | Diethylstilbestrol |
| L1300-05 | Ligand-66 | Valsartan |
| L1300-05 | Ligand-57 | Nicotinic Acid |
| L1300-05 | Ligand-38 | Tetrabenazine (Xenazine) |
| L1300-05 | Ligand-23 | Methylprednisolone |
| L1300-06 | Ligand-74 | Flunarizine 2HCl |
| L1300-06 | Ligand-63 | Uridine |
| L1300-06 | Ligand-51 | Sulindac |
| L1300-06 | Ligand-14 | Phenindione |
| L1300-06 | Ligand-59 | Sulbactam |
| L1300-06 | Ligand-10 | Memantine HCl |
| L1300-06 | Ligand-68 | Sulfisoxazole |
| L1300-06 | Ligand-9 | Rebamipide |
| L1300-06 | Ligand-24 | Tioconazole |
| L1300-06 | Ligand-83 | Lacidipine |
| L1300-06 | Ligand-69 | Isoniazid |
| L1300-06 | Ligand-42 | Candesartan Cilexetil |
| L1300-06 | Ligand-26 | Methoxsalen |
| L1300-06 | Ligand-31 | Aspartame |
| L1300-06 | Ligand-52 | Diclazuril |
| L1300-06 | Ligand-70 | Tolfenamic Acid |
| L1300-06 | Ligand-88 | Balofloxacin |
| L1300-06 | Ligand-61 | Propylthiouracil |
| L1300-06 | Ligand-40 | Pyrimethamine |
| L1300-06 | Ligand-21 | Cyproheptadine HCl |
| L1300-06 | Ligand-22 | Cyclophosphamide Monohydrate |
| L1300-06 | Ligand-27 | Primidone |
| L1300-06 | Ligand-67 | Amlodipine |
| L1300-06 | Ligand-17 | Methscopolamine |
| L1300-06 | Ligand-7 | Elvitegravir (GS-9137, JTK-303) |
| L1300-06 | Ligand-75 | Dyclonine HCl |
| L1300-06 | Ligand-15 | Menadione |
| L1300-06 | Ligand-11 | Gimeracil |
| L1300-06 | Ligand-39 | Meclizine 2HCl |
| L1300-06 | Ligand-3 | Haloperidol |
| L1300-06 | Ligand-64 | Nimesulide |
| L1300-06 | Ligand-13 | Fluvastatin Sodium |
| L1300-06 | Ligand-33 | Tolnaftate |
| L1300-06 | Ligand-80 | Levofloxacin |
| L1300-06 | Ligand-30 | Ketotifen Fumarate |
| L1300-06 | Ligand-77 | Tiopronin |
| L1300-06 | Ligand-54 | Lornoxicam |
| L1300-06 | Ligand-71 | Meglumine |
| L1300-06 | Ligand-85 | Fenticonazole Nitrate |
| L1300-06 | Ligand-2 | Flutamide |
| L1300-06 | Ligand-16 | Rimantadine |
| L1300-06 | Ligand-65 | Captopril |
| L1300-06 | Ligand-57 | Sulfamethoxazole |
| L1300-06 | Ligand-49 | Nicorandil |
| L1300-06 | Ligand-4 | Enalapril Maleate |
| L1300-06 | Ligand-8 | Formoterol Hemifumarate |
| L1300-06 | Ligand-84 | Benidipine HCl |
| L1300-06 | Ligand-32 | Doxifluridine |
| L1300-06 | Ligand-66 | Lovastatin |
| L1300-06 | Ligand-76 | Oxytetracycline Dihydrate |
| L1300-06 | Ligand-28 | Amiodarone HCl |
| L1300-06 | Ligand-19 | Chlormezanone |
| L1300-06 | Ligand-41 | Urapidil HCl |
| L1300-06 | Ligand-48 | Sulfamethizole |
| L1300-06 | Ligand-35 | Tropicamide |
| L1300-06 | Ligand-25 | Alibendol |
| L1300-06 | Ligand-38 | Nefiracetam |
| L1300-06 | Ligand-47 | Triamcinolone |
| L1300-06 | Ligand-18 | Maraviroc |
| L1300-06 | Ligand-73 | Mirtazapine |
| L1300-06 | Ligand-37 | Miconazole Nitrate |
| L1300-06 | Ligand-53 | Phentolamine Mesylate |
| L1300-06 | Ligand-23 | Nicotinamide (Vitamin B3) |
| L1300-06 | Ligand-6 | Sarafloxacin HCl |
| L1300-06 | Ligand-20 | Epalrestat |
| L1300-06 | Ligand-50 | Mometasone furoate |
| L1300-06 | Ligand-86 | Cyproterone Acetate |
| L1300-06 | Ligand-36 | Irsogladine |
| L1300-06 | Ligand-1 | Potassium Iodide |
| L1300-06 | Ligand-60 | Tamoxifen Citrate |
| L1300-06 | Ligand-45 | Diclofenac Sodium |
| L1300-06 | Ligand-87 | Orphenadrine Citrate |
| L1300-06 | Ligand-78 | Metronidazole |
| L1300-06 | Ligand-62 | Pramipexole dihydrochloride monohydrate |
| L1300-06 | Ligand-12 | Tropisetron |
| L1300-06 | Ligand-5 | Sulphadimethoxine |
| L1300-06 | Ligand-79 | Crystal Violet |
| L1300-06 | Ligand-55 | Bromhexine HCl |
| L1300-06 | Ligand-29 | Raltegravir (MK-0518) |
| L1300-06 | Ligand-58 | Nystatin (Fungicidin) |
| L1300-06 | Ligand-34 | Vitamin B12 |
| L1300-06 | Ligand-82 | Aripiprazole |
| L1300-06 | Ligand-72 | Fluticasone propionate |
| L1300-06 | Ligand-46 | Pregnenolone |
| L1300-06 | Ligand-56 | Avobenzone |
| L1300-06 | Ligand-81 | Pranoprofen |
| L1300-06 | Ligand-43 | Pioglitazone HCl |
| L1300-06 | Ligand-44 | Terazosin HCl |
| L1300-07 | Ligand-17 | S-(+)-Rolipram |
| L1300-07 | Ligand-24 | Cilazapril Monohydrate |
| L1300-07 | Ligand-16 | Conivaptan HCl |
| L1300-07 | Ligand-11 | Tolbutamide |
| L1300-07 | Ligand-35 | Adiphenine HCl |
| L1300-07 | Ligand-29 | Tebipenem Pivoxil |
| L1300-07 | Ligand-3 | Dexmedetomidine HCl (Precedex) |
| L1300-07 | Ligand-14 | Betaxolol |
| L1300-07 | Ligand-61 | Neratinib (HKI-272) |
| L1300-07 | Ligand-13 | Clevidipine Butyrate |
| L1300-07 | Ligand-22 | Levosimendan |
| L1300-07 | Ligand-52 | Irinotecan HCl Trihydrate |
| L1300-07 | Ligand-20 | Esomeprazole Sodium |
| L1300-07 | Ligand-72 | LDE225 (NVP-LDE225, Erismodegib) |
| L1300-07 | Ligand-34 | Argatroban |
| L1300-07 | Ligand-19 | R788 (Fostamatinib) Disodium |
| L1300-07 | Ligand-30 | Formestane |
| L1300-07 | Ligand-8 | Aliskiren Hemifumarate |
| L1300-07 | Ligand-33 | Amantadine HCl |
| L1300-07 | Ligand-57 | Trimebutine |
| L1300-07 | Ligand-77 | Bethanechol chloride |
| L1300-07 | Ligand-49 | Licofelone |
| L1300-07 | Ligand-28 | Bazedoxifene HCl |
| L1300-07 | Ligand-85 | Eltrombopag Olamine |
| L1300-07 | Ligand-38 | Probucol |
| L1300-07 | Ligand-56 | Rosiglitazone HCl |
| L1300-07 | Ligand-4 | Rasagiline Mesylate |
| L1300-07 | Ligand-82 | Mestranol |
| L1300-07 | Ligand-67 | Atorvastatin Calcium |
| L1300-07 | Ligand-27 | Ibutilide Fumarate |
| L1300-07 | Ligand-65 | D-Mannitol |
| L1300-07 | Ligand-36 | Almotriptan Malate |
| L1300-07 | Ligand-25 | Detomidine HCl |
| L1300-07 | Ligand-83 | Sitafloxacin Hydrate |
| L1300-07 | Ligand-45 | Mecarbinate |
| L1300-07 | Ligand-60 | Dextrose |
| L1300-07 | Ligand-10 | Nalidixic acid |
| L1300-07 | Ligand-7 | Dabigatran Etexilate |
| L1300-07 | Ligand-58 | Bexarotene |
| L1300-07 | Ligand-23 | Ozagrel HCl |
| L1300-07 | Ligand-70 | Lapatinib |
| L1300-07 | Ligand-88 | Chlorpromazine HCl |
| L1300-07 | Ligand-32 | Piperine |
| L1300-07 | Ligand-59 | Vinpocetine |
| L1300-07 | Ligand-78 | Famotidine |
| L1300-07 | Ligand-18 | Taladegib (LY2940680) |
| L1300-07 | Ligand-46 | Duloxetine HCl |
| L1300-07 | Ligand-42 | Fesoterodine Fumarate |
| L1300-07 | Ligand-55 | Benserazide HCl |
| L1300-07 | Ligand-80 | Gabexate Mesylate |
| L1300-07 | Ligand-9 | Galunisertib (LY2157299) |
| L1300-07 | Ligand-73 | Abemaciclib (LY2784544) |
| L1300-07 | Ligand-81 | Cisatracurium Besylate |
| L1300-07 | Ligand-76 | L-carnitine |
| L1300-07 | Ligand-2 | Moexipril HCl |
| L1300-07 | Ligand-53 | CX-4945 (Silmitasertib) |
| L1300-07 | Ligand-41 | Mubritinib (TAK 165) |
| L1300-07 | Ligand-68 | Ivabradine HCl |
| L1300-07 | Ligand-47 | Ambrisentan |
| L1300-07 | Ligand-79 | Rivastigmine Tartrate |
| L1300-07 | Ligand-1 | Lafutidine |
| L1300-07 | Ligand-69 | Temocapril HCl |
| L1300-07 | Ligand-6 | Naftopidil |
| L1300-07 | Ligand-71 | Xylose |
| L1300-07 | Ligand-39 | Atropine |
| L1300-07 | Ligand-86 | DL-Carnitine HCl |
| L1300-07 | Ligand-12 | Moxonidine |
| L1300-07 | Ligand-51 | Rosuvastatin Calcium |
| L1300-07 | Ligand-37 | Flunixin Meglumin |
| L1300-07 | Ligand-64 | Artemether |
| L1300-07 | Ligand-43 | Rutin |
| L1300-07 | Ligand-5 | Dronedarone HCl |
| L1300-07 | Ligand-84 | MLN2238 |
| L1300-07 | Ligand-21 | Orotic acid (6-Carboxyuracil) |
| L1300-07 | Ligand-63 | Apatinib |
| L1300-07 | Ligand-44 | Amfebutamone HCl |
| L1300-07 | Ligand-40 | Bazedoxifene Acetate |
| L1300-07 | Ligand-26 | Levosulpiride |
| L1300-07 | Ligand-74 | CAL-101 (Idelalisib, GS-1101) |
| L1300-07 | Ligand-54 | Silibinin |
| L1300-07 | Ligand-31 | Volasertib (BI 6727) |
| L1300-07 | Ligand-75 | Cyclosporin A |
| L1300-07 | Ligand-15 | Naltrexone HCl |
| L1300-07 | Ligand-87 | Sorbitol |
| L1300-07 | Ligand-66 | Bupivacaine HCl |
| L1300-07 | Ligand-48 | Imidapril HCl |
| L1300-07 | Ligand-50 | Roflumilast |
| L1300-07 | Ligand-62 | Telotristat Etiprate (LX 1606 |
| | | Hippurate) |
| L1300-08 | Ligand-53 | Clomifene citrate |
| L1300-08 | Ligand-29 | Econazole nitrate |
| L1300-08 | Ligand-72 | Ciclopirox |
| L1300-08 | Ligand-18 | Isoconazole nitrate |
| L1300-08 | Ligand-39 | L-Adrenaline |
| L1300-08 | Ligand-75 | Amoxicillin Sodium |
| L1300-08 | Ligand-69 | Olanzapine |
| L1300-08 | Ligand-28 | Epinephrine Bitartrate |
| L1300-08 | Ligand-86 | Isoprenaline HCl |
| L1300-08 | Ligand-58 | Novobiocin Sodium |
| L1300-08 | Ligand-16 | Scopolamine HBr |
| L1300-08 | Ligand-27 | Sotalol |
| L1300-08 | Ligand-14 | Moroxydine HCl |
| L1300-08 | Ligand-26 | Pancuronium dibromide |
| L1300-08 | Ligand-15 | Ozagrel |
| L1300-08 | Ligand-13 | Imatinib (STI571) |
| L1300-08 | Ligand-54 | Tetracycline HCl |
| L1300-08 | Ligand-8 | Ceftiofur HCl |
| L1300-08 | Ligand-37 | Phenoxybenzamine HCl |
| L1300-08 | Ligand-32 | Prednisolone Acetate |
| L1300-08 | Ligand-30 | Trospium chloride |
| L1300-08 | Ligand-88 | Pioglitazone |
| L1300-08 | Ligand-84 | Phenformin HCl |
| L1300-08 | Ligand-33 | Thiamphenicol |
| L1300-08 | Ligand-2 | Gallamine Triethiodide |
| L1300-08 | Ligand-11 | Quinapril HCl |
| L1300-08 | Ligand-36 | Nateglinide |
| L1300-08 | Ligand-21 | Phenylephrine HCl |
| L1300-08 | Ligand-24 | Itraconazole |
| L1300-08 | Ligand-25 | Mycophenolic acid |
| L1300-08 | Ligand-22 | Trazodone HCl |
| L1300-08 | Ligand-38 | Spectinomycin HCl |
| L1300-08 | Ligand-31 | Cortisone acetate |
| L1300-08 | Ligand-19 | Tiotropium Bromide hydrate |
| L1300-08 | Ligand-17 | Naphazoline HCl |
| L1300-08 | Ligand-10 | Medroxyprogesterone acetate |
| L1300-08 | Ligand-60 | Tenoxicam |
| L1300-08 | Ligand-5 | Roxithromycin |
| L1300-08 | Ligand-42 | Amiloride hydrochloride dihydrate |
| L1300-08 | Ligand-7 | Ritodrine HCl |
| L1300-08 | Ligand-6 | Maprotiline HCl |
| L1300-08 | Ligand-34 | Pramipexole |
| L1300-08 | Ligand-78 | Fluocinolone Acetonide |
| L1300-08 | Ligand-52 | Sulbactam sodium |
| L1300-08 | Ligand-68 | Manidipine 2HCl |
| L1300-08 | Ligand-12 | Clonidine HCl |
| L1300-08 | Ligand-46 | Loperamide HCl |
| L1300-08 | Ligand-73 | Clomipramine HCl |
| L1300-08 | Ligand-35 | Lincomycin HCl |
| L1300-08 | Ligand-4 | Oxymetazoline hydrochloride |
| L1300-08 | Ligand-57 | Manidipine |
| L1300-08 | Ligand-40 | Miconazole |
| L1300-08 | Ligand-41 | Tolterodine tartrate |
| L1300-08 | Ligand-48 | Propafenone HCl |
| L1300-08 | Ligand-51 | Secnidazole |
| L1300-08 | Ligand-56 | Estriol |
| L1300-08 | Ligand-43 | Tetracaine HCl |
| L1300-08 | Ligand-44 | Clobetasol propionate |
| L1300-08 | Ligand-1 | Clindamycin HCl |
| L1300-08 | Ligand-50 | Phenytoin sodium |
| L1300-08 | Ligand-20 | Terbinafine HCl |
| L1300-08 | Ligand-63 | Azelastine HCl |
| L1300-08 | Ligand-66 | Dimethyl Fumarate |
| L1300-08 | Ligand-67 | Famciclovir |
| L1300-08 | Ligand-55 | Brompheniramine hydrogen maleate |
| L1300-08 | Ligand-45 | Domperidone |
| L1300-08 | Ligand-76 | Phenacetin |
| L1300-08 | Ligand-87 | Zidovudine |
| L1300-08 | Ligand-81 | Rosiglitazone maleate |
| L1300-08 | Ligand-70 | Ribavirin |
| L1300-08 | Ligand-64 | Cloxacillin Sodium |
| L1300-08 | Ligand-65 | Xylometazoline HCl |
| L1300-08 | Ligand-59 | Racecadotril |
| L1300-08 | Ligand-23 | Clozapine |
| L1300-08 | Ligand-61 | Phenytoin |
| L1300-08 | Ligand-77 | Miglitol |
| L1300-08 | Ligand-82 | Xylazine HCl |
| L1300-08 | Ligand-85 | Clarithromycin |
| L1300-08 | Ligand-47 | Nitrendipine |
| L1300-08 | Ligand-9 | Rosiglitazone |
| L1300-08 | Ligand-71 | Vardenafil HCl Trihydrate |
| L1300-08 | Ligand-49 | Sulfadoxine |
| L1300-08 | Ligand-79 | Milrinone |
| L1300-08 | Ligand-74 | 5-Aminolevulinic acid HCl |
| L1300-08 | Ligand-80 | Olopatadine HCl |
| L1300-08 | Ligand-62 | Acetanilide |
| L1300-08 | Ligand-3 | Mitoxantrone HCl |
| L1300-08 | Ligand-83 | Dopamine HCl |
| L1300-09 | Ligand-37 | Tivantinib (ARQ 197) |
| L1300-09 | Ligand-51 | Rivaroxaban |
| L1300-09 | Ligand-26 | TG101348 (SAR302503) |
| L1300-09 | Ligand-14 | GSK2126458 (GSK458) |
| L1300-09 | Ligand-10 | Cinepazide maleate |
| L1300-09 | Ligand-39 | Alogliptin |
| L1300-09 | Ligand-49 | Lonafarnib |
| L1300-09 | Ligand-38 | Sofosbuvir (PSI-7977, GS-7977) |
| L1300-09 | Ligand-8 | Amoxicillin |
| L1300-09 | Ligand-88 | Chlorhexidine HCl |
| L1300-09 | Ligand-50 | Camostat Mesilate |
| L1300-09 | Ligand-79 | Noradrenaline bitartrate monohydrate |
| L1300-09 | Ligand-66 | Ambroxol HCl |
| L1300-09 | Ligand-44 | Diclofenac Potassium |
| L1300-09 | Ligand-78 | Tioxolone |
| L1300-09 | Ligand-18 | Evacetrapib (LY2484595) |
| L1300-09 | Ligand-46 | Lonidamine |
| L1300-09 | Ligand-30 | Niflumic acid |
| L1300-09 | Ligand-11 | Enrofloxacin |
| L1300-09 | Ligand-83 | S-Ruxolitinib (INCB018424) |
| L1300-09 | Ligand-21 | Azilsartan |
| L1300-09 | Ligand-75 | Fosaprepitant dimeglumine salt |
| L1300-09 | Ligand-81 | Dinaciclib (SCH727965) |
| L1300-09 | Ligand-41 | Ciclopirox ethanolamine |
| L1300-09 | Ligand-67 | Acemetacin |
| L1300-09 | Ligand-56 | Gliclazide |
| L1300-09 | Ligand-74 | Bufexamac |
| L1300-09 | Ligand-82 | BYL719 |
| L1300-09 | Ligand-13 | Mifepristone |
| L1300-09 | Ligand-85 | Lamotrigine |
| L1300-09 | Ligand-40 | Clevudine |
| L1300-09 | Ligand-12 | Pramiracetam |
| L1300-09 | Ligand-72 | Cobicistat (GS-9350) |
| L1300-09 | Ligand-55 | Diclofenac Diethylamine |
| L1300-09 | Ligand-34 | Oseltamivir Phosphate |
| L1300-09 | Ligand-3 | Fostamatinib (R788) |
| L1300-09 | Ligand-68 | Arecoline |
| L1300-09 | Ligand-43 | Bosentan Hydrate |
| L1300-09 | Ligand-22 | Medetomidine HCl |
| L1300-09 | Ligand-28 | Carfilzomib (PR-171) |
| L1300-09 | Ligand-9 | Linagliptin |
| L1300-09 | Ligand-86 | Rofecoxib |
| L1300-09 | Ligand-60 | Galeterone |
| L1300-09 | Ligand-45 | L-Thyroxine |
| L1300-09 | Ligand-71 | Dabrafenib (GSK2118436) |
| L1300-09 | Ligand-80 | Nilvadipine |
| L1300-09 | Ligand-19 | Aspirin |
| L1300-09 | Ligand-35 | Fluocinonide |
| L1300-09 | Ligand-17 | Clindamycin |
| L1300-09 | Ligand-33 | Epinephrine HCl |
| L1300-09 | Ligand-87 | Azilsartan Medoxomil |
| L1300-09 | Ligand-32 | Otilonium Bromide |
| L1300-09 | Ligand-16 | Tofacitinib (CP-690550, Tasocitinib) |
| L1300-09 | Ligand-6 | Tideglusib |
| L1300-09 | Ligand-15 | Crenolanib (CP-868596) |
| L1300-09 | Ligand-31 | Vildagliptin (LAF-237) |
| L1300-09 | Ligand-42 | Daunorubicin HCl |
| L1300-09 | Ligand-27 | Sotrastaurin |
| L1300-09 | Ligand-54 | Rupatadine Fumarate |
| L1300-09 | Ligand-5 | Dovitinib (TKI-258) Dilactic Acid |
| L1300-09 | Ligand-76 | Alverine Citrate |
| L1300-09 | Ligand-70 | Canagliflozin |
| L1300-09 | Ligand-2 | Idebenone |
| L1300-09 | Ligand-62 | Paroxetine HCl |
| L1300-09 | Ligand-53 | Pravastatin sodium |
| L1300-09 | Ligand-57 | Clorsulon |
| L1300-09 | Ligand-59 | TH-302 |
| L1300-09 | Ligand-24 | Buflomedil HCl |
| L1300-09 | Ligand-65 | Azelnidipine |
| L1300-09 | Ligand-47 | Trametinib (GSK1120212) |
| L1300-09 | Ligand-52 | Rimonabant |
| L1300-09 | Ligand-58 | Ibrutinib (PCI-32765) |
| L1300-09 | Ligand-48 | Varlitinib |
| L1300-09 | Ligand-7 | Pirfenidone |
| L1300-09 | Ligand-4 | Dacomitinib (PF299804, PF299) |
| L1300-09 | Ligand-29 | Carbazochrome sodium sulfonate (AC-17) |
| L1300-09 | Ligand-64 | Bepotastine Besilate |
| L1300-09 | Ligand-73 | Zaltoprofen |
| L1300-09 | Ligand-77 | Naloxone HCl |
| L1300-09 | Ligand-25 | Ciprofibrate |
| L1300-09 | Ligand-36 | Dolutegravir (GSK1349572) |
| L1300-09 | Ligand-20 | Bindarit |
| L1300-09 | Ligand-1 | Tolvaptan |
| L1300-09 | Ligand-61 | Prucalopride |
| L1300-09 | Ligand-63 | Cabazitaxel |
| L1300-09 | Ligand-69 | Resminostat |
| L1300-09 | Ligand-23 | Clindamycin palmitate HCl |
| L1300-09 | Ligand-84 | Pazopanib |
| L1300-10 | Ligand-32 | Avanafil |
| L1300-10 | Ligand-19 | Trifluoperazine 2HCl |
| L1300-10 | Ligand-16 | Mepivacaine HCl |
| L1300-10 | Ligand-26 | Milnacipran HCl |
| L1300-10 | Ligand-81 | Gliquidone |
| L1300-10 | Ligand-38 | Sertaconazole nitrate |
| L1300-10 | Ligand-41 | Fexofenadine HCl |
| L1300-10 | Ligand-36 | Trimethoprim |
| L1300-10 | Ligand-51 | Ropinirole HCl |
| L1300-10 | Ligand-70 | Articaine HCl |
| L1300-10 | Ligand-49 | Tylosin tartrate |
| L1300-10 | Ligand-60 | Benztropine mesylate |
| L1300-10 | Ligand-47 | Biotin (Vitamin B7) |
| L1300-10 | Ligand-72 | Eletriptan HBr |
| L1300-10 | Ligand-53 | Mirabegron |
| L1300-10 | Ligand-61 | Carbenicillin disodium |
| L1300-10 | Ligand-15 | Methenamine |
| L1300-10 | Ligand-75 | Ampiroxicam |
| L1300-10 | Ligand-68 | Vitamin C |
| L1300-10 | Ligand-35 | Nadifloxacin |
| L1300-10 | Ligand-24 | Moguisteine |
| L1300-10 | Ligand-57 | Pyridoxine HCl |
| L1300-10 | Ligand-40 | (+,−)-Octopamine HCl |
| L1300-10 | Ligand-69 | Sulfamethazine |
| L1300-10 | Ligand-73 | Azacyclonol |
| L1300-10 | Ligand-86 | Desloratadine |
| L1300-10 | Ligand-37 | Darifenacin HBr |
| L1300-10 | Ligand-14 | Ornidazole |
| L1300-10 | Ligand-62 | Azlocillin sodium salt |
| L1300-10 | Ligand-28 | Atomoxetine HCl |
| L1300-10 | Ligand-64 | Acebutolol HCl |
| L1300-10 | Ligand-65 | Probenecid |
| L1300-10 | Ligand-30 | Meptazinol HCl |
| L1300-10 | Ligand-29 | Azatadine dimaleate |
| L1300-10 | Ligand-79 | Sulfathiazole |
| L1300-10 | Ligand-46 | Pidotimod |
| L1300-10 | Ligand-42 | Pentamidine |
| L1300-10 | Ligand-59 | Estradiol valerate |
| L1300-10 | Ligand-43 | Sodium Picosulfate |
| L1300-10 | Ligand-17 | Antipyrine |
| L1300-10 | Ligand-20 | Ethambutol HCl |
| L1300-10 | Ligand-25 | Dexamethasone Acetate |
| L1300-10 | Ligand-77 | Dibucaine HCl |
| L1300-10 | Ligand-74 | Cabozantinib malate (XL184) |
| L1300-10 | Ligand-55 | Vitamin D2 |
| L1300-10 | Ligand-58 | Sulfamerazine |
| L1300-10 | Ligand-12 | Caspofungin Acetate |
| L1300-10 | Ligand-39 | Betahistine 2HCl |
| L1300-10 | Ligand-18 | Adrenalone HCl |
| L1300-10 | Ligand-78 | Etravirine (TMC125) |
| L1300-10 | Ligand-52 | Moclobemide (Ro111163) |
| L1300-10 | Ligand-87 | Homatropine Methylbromide |
| L1300-10 | Ligand-67 | Atovaquone |
| L1300-10 | Ligand-9 | Lithocholic acid |
| L1300-10 | Ligand-88 | Methazolamide |
| L1300-10 | Ligand-21 | Allylthiourea |
| L1300-10 | Ligand-10 | Hyoscyamine |
| L1300-10 | Ligand-76 | Procaine HCl |
| L1300-10 | Ligand-66 | Doxapram HCl |
| L1300-10 | Ligand-63 | Pergolide mesylate |
| L1300-10 | Ligand-80 | Sodium salicylate |
| L1300-10 | Ligand-5 | Butenafine HCl |
| L1300-10 | Ligand-23 | Dexmedetomidine |
| L1300-10 | Ligand-83 | Flumequine |
| L1300-10 | Ligand-82 | Ampicillin sodium |
| L1300-10 | Ligand-44 | Diphemanil Methylsulfate |
| L1300-10 | Ligand-54 | Tolcapone |
| L1300-10 | Ligand-50 | Brinzolamide |
| L1300-10 | Ligand-27 | Ethynodiol diacetate |
| L1300-10 | Ligand-11 | Homatropine Bromide |
| L1300-10 | Ligand-85 | Sitagliptin phosphate monohydrate |
| L1300-10 | Ligand-33 | Aclidinium Bromide |
| L1300-10 | Ligand-3 | Oxybutynin chloride |
| L1300-10 | Ligand-8 | Triflusal |
| L1300-10 | Ligand-34 | Foscarnet Sodium |
| L1300-10 | Ligand-56 | Beclomethasone dipropionate |
| L1300-10 | Ligand-45 | Tazobactam |
| L1300-10 | Ligand-4 | Methylthiouracil |
| L1300-10 | Ligand-2 | Ulipristal |
| L1300-10 | Ligand-22 | Hydroxyzine 2HCl |
| L1300-10 | Ligand-1 | Piracetam |
| L1300-10 | Ligand-6 | Anagrelide HCl |
| L1300-10 | Ligand-48 | Entacapone |
| L1300-10 | Ligand-7 | Amitriptyline HCl |
| L1300-10 | Ligand-84 | Reboxetine mesylate |
| L1300-10 | Ligand-71 | Altrenogest |
| L1300-10 | Ligand-31 | Doxycycline HCl |
| L1300-10 | Ligand-13 | Indacaterol Maleate |
| L1300-11 | Ligand-69 | Escitalopram Oxalate |
| L1300-11 | Ligand-70 | Zinc Pyrithione |
| L1300-11 | Ligand-86 | Cyclizine 2HCl |
| L1300-11 | Ligand-59 | Sodium Nitrite |
| L1300-11 | Ligand-80 | Guanabenz Acetate |
| L1300-11 | Ligand-64 | Carprofen |
| L1300-11 | Ligand-48 | Sodium 4-Aminosalicylate |
| L1300-11 | Ligand-51 | Eprosartan Mesylate |
| L1300-11 | Ligand-58 | Vitamin D3 |
| L1300-11 | Ligand-25 | Erythromycin Ethylsuccinate |
| L1300-11 | Ligand-53 | Clorprenaline HCl |
| L1300-11 | Ligand-27 | Ticagrelor |
| L1300-11 | Ligand-40 | Voglibose |
| L1300-11 | Ligand-54 | Ampicillin Trihydrate |
| L1300-11 | Ligand-81 | Propranolol HCl |
| L1300-11 | Ligand-65 | Amfenac Sodium Monohydrate |
| L1300-11 | Ligand-75 | Dropropizine |
| L1300-11 | Ligand-79 | Methyclothiazide |
| L1300-11 | Ligand-67 | Estradiol Cypionate |
| L1300-11 | Ligand-68 | Retapamulin |
| L1300-11 | Ligand-41 | Desvenlafaxine |
| L1300-11 | Ligand-77 | Doxofylline |
| L1300-11 | Ligand-84 | Clofazimine |
| L1300-11 | Ligand-12 | Olsalazine Sodium |
| L1300-11 | Ligand-61 | Difluprednate |
| L1300-11 | Ligand-72 | Droperidol |
| L1300-11 | Ligand-28 | Halobetasol Propionate |
| L1300-11 | Ligand-30 | Desvenlafaxine Succinate |
| L1300-11 | Ligand-62 | Closantel Sodium |
| L1300-11 | Ligand-42 | Troxipide |
| L1300-11 | Ligand-6 | Loxapine Succinate |
| L1300-11 | Ligand-63 | Histamine 2HCl |
| L1300-11 | Ligand-73 | Closantel |
| L1300-11 | Ligand-31 | Sodium Phenylbutyrate |
| L1300-11 | Ligand-47 | Ronidazole |
| L1300-11 | Ligand-76 | Penfluridol |
| L1300-11 | Ligand-45 | Toltrazuril |
| L1300-11 | Ligand-39 | Fenspiride HCl |
| L1300-11 | Ligand-88 | Benzydamine HCl |
| L1300-11 | Ligand-7 | Halcinonide |
| L1300-11 | Ligand-74 | Sulconazole Nitrate |
| L1300-11 | Ligand-29 | Esmolol HCl |
| L1300-11 | Ligand-5 | Mequinol |
| L1300-11 | Ligand-60 | Spiramycin |
| L1300-11 | Ligand-71 | Lomerizine HCl |
| L1300-11 | Ligand-83 | Dydrogesterone |
| L1300-11 | Ligand-10 | Dinitolmide |
| L1300-11 | Ligand-13 | Valdecoxib |
| L1300-11 | Ligand-82 | Levobetaxolol HCl |
| L1300-11 | Ligand-78 | Bisacodyl |
| L1300-11 | Ligand-52 | Triclabendazole |
| L1300-11 | Ligand-49 | Sulfacetamide Sodium |
| L1300-11 | Ligand-22 | Chlortetracycline HCl |
| L1300-11 | Ligand-9 | Timolol Maleate |
| L1300-11 | Ligand-56 | Pheniramine Maleate |
| L1300-11 | Ligand-38 | Triamterene |
| L1300-11 | Ligand-34 | Tetrahydrozoline HCl |
| L1300-11 | Ligand-33 | Bezafibrate |
| L1300-11 | Ligand-46 | Sertraline HCl |
| L1300-11 | Ligand-8 | Estradiol Benzoate |
| L1300-11 | Ligand-3 | Ropivacaine HCl |
| L1300-11 | Ligand-44 | Penicillin G Sodium |
| L1300-11 | Ligand-16 | Mefenamic Acid |
| L1300-11 | Ligand-55 | Benzoic Acid |
| L1300-11 | Ligand-1 | Norethindrone |
| L1300-11 | Ligand-37 | Decamethonium Bromide |
| L1300-11 | Ligand-4 | Tinidazole |
| L1300-11 | Ligand-17 | Flumethasone |
| L1300-11 | Ligand-11 | Chlorzoxazone |
| L1300-11 | Ligand-87 | Ethamsylate |
| L1300-11 | Ligand-24 | Valganciclovir HCl |
| L1300-11 | Ligand-57 | Spironolactone |
| L1300-11 | Ligand-36 | Levobupivacaine HCl |
| L1300-11 | Ligand-43 | Azithromycin Dihydrate |
| L1300-11 | Ligand-18 | Dexlansoprazole |
| L1300-11 | Ligand-35 | Nabumetone |
| L1300-11 | Ligand-2 | Carbimazole |
| L1300-11 | Ligand-23 | Nafcillin Sodium |
| L1300-11 | Ligand-20 | Tolazoline HCl |
| L1300-11 | Ligand-19 | Dicloxacillin Sodium |
| L1300-11 | Ligand-32 | Bacitracin |
| L1300-11 | Ligand-50 | Pramoxine HCl |
| L1300-11 | Ligand-26 | Griseofulvin |
| L1300-11 | Ligand-66 | Benzethonium Chloride |
| L1300-11 | Ligand-85 | Tilmicosin |
| L1300-11 | Ligand-15 | Guanidine HCl |
| L1300-11 | Ligand-21 | Clopidol |
| L1300-11 | Ligand-14 | Sodium Nitroprusside |
| L1300-12 | Ligand-14 | Cinchophen |
| L1300-12 | Ligand-15 | Tolperisone HCl |
| L1300-12 | Ligand-16 | Benzocaine |
| L1300-12 | Ligand-26 | Florfenicol |
| L1300-12 | Ligand-24 | Mezlocillin Sodium |
| L1300-12 | Ligand-9 | Diacerein |
| L1300-12 | Ligand-68 | Tiratricol |
| L1300-12 | Ligand-29 | Phenazopyridine HCl |
| L1300-12 | Ligand-30 | Sulfamethoxypyridazine |
| L1300-12 | Ligand-25 | Betamipron |
| L1300-12 | Ligand-4 | Antazoline HCl |
| L1300-12 | Ligand-33 | Procainamide HCl |
| L1300-12 | Ligand-51 | Cetrimonium Bromide (CTAB) |
| L1300-12 | Ligand-62 | Deoxycorticosterone acetate |
| L1300-12 | Ligand-59 | Cysteamine HCl |
| L1300-12 | Ligand-3 | Cyclandelate |
| L1300-12 | Ligand-47 | Broxyquinoline |
| L1300-12 | Ligand-79 | Domiphen Bromide |
| L1300-12 | Ligand-28 | Piperacillin Sodium |
| L1300-12 | Ligand-37 | Furaltadone HCl |
| L1300-12 | Ligand-11 | Diphenidol HCl |
| L1300-12 | Ligand-8 | Prucalopride Succinat |
| L1300-12 | Ligand-50 | Mexiletine HCl |
| L1300-12 | Ligand-5 | Chlorocresol |
| L1300-12 | Ligand-40 | Doxylamine Succinate |
| L1300-12 | Ligand-7 | Oxaprozin |
| L1300-12 | Ligand-12 | Cyromazine |
| L1300-12 | Ligand-85 | Ebastine |
| L1300-12 | Ligand-86 | Nelfinavir Mesylate |
| L1300-12 | Ligand-75 | Demeclocycline HCl |
| L1300-12 | Ligand-2 | Uracil |
| L1300-12 | Ligand-32 | Anidulafungin (LY303366) |
| L1300-12 | Ligand-84 | Tranylcypromine (2-PCPA) HCl |
| L1300-12 | Ligand-69 | Bemegride |
| L1300-12 | Ligand-77 | (R)-(+)-Atenolol |
| L1300-12 | Ligand-46 | Nifuroxazide |
| L1300-12 | Ligand-10 | Cyclobenzaprine HCl |
| L1300-12 | Ligand-6 | Bosentan |
| L1300-12 | Ligand-22 | Promethazine HCl |
| L1300-12 | Ligand-41 | Epinastine HCl |
| L1300-12 | Ligand-18 | Zoxazolamine |
| L1300-12 | Ligand-76 | Digoxin |
| L1300-12 | Ligand-27 | Montelukast Sodium |
| L1300-12 | Ligand-38 | Dirithromycin |
| L1300-12 | Ligand-49 | Valnemulin HCl |
| L1300-12 | Ligand-13 | Climbazole |
| L1300-12 | Ligand-48 | Isosorbide |
| L1300-12 | Ligand-83 | Oxybuprocaine HCl |
| L1300-12 | Ligand-81 | Chromocarb |
| L1300-12 | Ligand-42 | Oxiracetam |
| L1300-12 | Ligand-1 | Chlorpropamide |
| L1300-12 | Ligand-44 | Meclofenamate Sodium |
| L1300-12 | Ligand-34 | Coumarin |
| L1300-12 | Ligand-36 | Chlorquinaldol |
| L1300-12 | Ligand-31 | Vinorelbine Tartrate |
| L1300-12 | Ligand-60 | Liothyronine Sodium |
| L1300-12 | Ligand-53 | Rotigotine |
| L1300-12 | Ligand-35 | Nicardipine HCl |
| L1300-12 | Ligand-39 | Mevastatin |
| L1300-12 | Ligand-70 | Clofibric Acid |
| L1300-12 | Ligand-64 | Carteolol HCl |
| L1300-12 | Ligand-57 | Penciclovir |
| L1300-12 | Ligand-88 | Anisotropine Methylbromide |
| L1300-12 | Ligand-71 | Amoxapine |
| L1300-12 | Ligand-19 | Bromfenac Sodium |
| L1300-12 | Ligand-72 | Fluorometholone Acetate |
| L1300-12 | Ligand-23 | Teriflunomide |
| L1300-12 | Ligand-20 | Flufenamic acid |
| L1300-12 | Ligand-55 | Salmeterol Xinafoate |
| L1300-12 | Ligand-43 | Micafungin Sodium |
| L1300-12 | Ligand-56 | Cetylpyridinium Chloride |
| L1300-12 | Ligand-65 | MetoclopraMide HCl |
| L1300-12 | Ligand-73 | Serotonin HCl |
| L1300-12 | Ligand-87 | Labetalol HCl |
| L1300-12 | Ligand-61 | Fidaxomicin |
| L1300-12 | Ligand-66 | Mupirocin |
| L1300-12 | Ligand-78 | Trometamol |
| L1300-12 | Ligand-45 | Choline Chloride |
| L1300-12 | Ligand-54 | Chloroambucil |
| L1300-12 | Ligand-63 | Luliconazole |
| L1300-12 | Ligand-74 | Tamibarotene |
| L1300-12 | Ligand-52 | Buspirone HCl |
| L1300-12 | Ligand-82 | Azaperone |
| L1300-12 | Ligand-67 | Sulfaguanidine |
| L1300-12 | Ligand-58 | Ethacridine lactate monohydrate |
| L1300-12 | Ligand-17 | Benzbromarone |
| L1300-12 | Ligand-80 | Aminothiazole |
| L1300-12 | Ligand-21 | Ospemifene |
| L1300-13 | Ligand-57 | Nialamide |
| L1300-13 | Ligand-2 | Metaproterenol Sulfate |
| L1300-13 | Ligand-4 | Noscapine HCl |
| L1300-13 | Ligand-35 | Moxalactam Disodium |
| L1300-13 | Ligand-72 | Sodium 4-aminohippurate Hydrate |
| L1300-13 | Ligand-16 | Proadifen HCl |
| L1300-13 | Ligand-67 | Mepiroxol |
| L1300-13 | Ligand-81 | Aceclidine HCl |
| L1300-13 | Ligand-39 | Oxeladin Citrate |
| L1300-13 | Ligand-86 | Pantoprazole sodium |
| L1300-13 | Ligand-56 | Isoetharine Mesylate |
| L1300-13 | Ligand-3 | Procyclidine HCl |
| L1300-13 | Ligand-28 | Nifenazone |
| L1300-13 | Ligand-73 | Aceglutamide |
| L1300-13 | Ligand-9 | Fenofibric acid |
| L1300-13 | Ligand-83 | Trimipramine Maleate |
| L1300-13 | Ligand-37 | Nicotine Ditartrate |
| L1300-13 | Ligand-84 | Acetylleucine |
| L1300-13 | Ligand-79 | Piromidic Acid |
| L1300-13 | Ligand-69 | Carbachol |
| L1300-13 | Ligand-5 | Imipramine HCl |
| L1300-13 | Ligand-23 | Diphenylpyraline HCl |
| L1300-13 | Ligand-12 | Carbadox |
| L1300-13 | Ligand-10 | Salicylic acid |
| L1300-13 | Ligand-20 | Furazolidone |
| L1300-13 | Ligand-31 | Idramantone |
| L1300-13 | Ligand-60 | Bephenium Hydroxynaphthoate |
| L1300-13 | Ligand-7 | Mefloquine HCl |
| L1300-13 | Ligand-85 | Fenbufen |
| L1300-13 | Ligand-74 | Ethylparaben |
| L1300-13 | Ligand-71 | Brucine |
| L1300-13 | Ligand-68 | Pentoxifylline |
| L1300-13 | Ligand-34 | Disopyramide Phosphate |
| L1300-13 | Ligand-18 | Eltrombopag |
| L1300-13 | Ligand-80 | Glafenine HCl |
| L1300-13 | Ligand-29 | 6-Mercaptopurine (6-MP) Monohydrate |
| L1300-13 | Ligand-46 | Nalmefene HCl |
| L1300-13 | Ligand-8 | (+)-Camphor |
| L1300-13 | Ligand-1 | Benzthiazide |
| L1300-13 | Ligand-87 | Azelaic acid |
| L1300-13 | Ligand-62 | 17-Hydroxyprogesterone |
| L1300-13 | Ligand-41 | Citric acid trilithium salt |
| | | tetrahydrate |
| L1300-13 | Ligand-14 | Ractopamine HCl |
| L1300-13 | Ligand-75 | Nitrofurantoin |
| L1300-13 | Ligand-70 | Mepenzolate Bromide |
| L1300-13 | Ligand-63 | Dehydroacetic acid |
| L1300-13 | Ligand-13 | Metaraminol Bitartrate |
| L1300-13 | Ligand-27 | Pyrilamine Maleate |
| L1300-13 | Ligand-49 | Fosfomycin Tromethamine |
| L1300-13 | Ligand-24 | Metricrane |
| L1300-13 | Ligand-6 | Diperodon HCl |
| L1300-13 | Ligand-47 | Tacrine HCl |
| L1300-13 | Ligand-61 | Procodazole |
| L1300-13 | Ligand-40 | Vinblastine sulfate |
| L1300-13 | Ligand-51 | Acetazolamide |
| L1300-13 | Ligand-48 | Dicyclomine HCl |
| L1300-13 | Ligand-42 | i-Inositol |
| L1300-13 | Ligand-58 | Pimozide |
| L1300-13 | Ligand-11 | Bithionol |
| L1300-13 | Ligand-50 | Pasiniazid |
| L1300-13 | Ligand-55 | Carzenide |
| L1300-13 | Ligand-15 | Phthalylsulfacetamide |
| L1300-13 | Ligand-44 | Carsalam |
| L1300-13 | Ligand-36 | Tolazamide |
| L1300-13 | Ligand-53 | Iopamidol |
| L1300-13 | Ligand-19 | Cefotaxime sodium |
| L1300-13 | Ligand-38 | Difloxacin HCl |
| L1300-13 | Ligand-45 | Ethoxzolamide |
| L1300-13 | Ligand-78 | Mesoridazine Besylate |
| L1300-13 | Ligand-43 | Trimetazidine dihydrochloride |
| L1300-13 | Ligand-82 | Clofoctol |
| L1300-13 | Ligand-54 | Urethane |
| L1300-13 | Ligand-59 | Thioridazine HCl |
| L1300-13 | Ligand-17 | Isoxicam |
| L1300-13 | Ligand-76 | Aminoguanidine (hydrochloride) |
| L1300-13 | Ligand-30 | Chloroxylenol |
| L1300-13 | Ligand-21 | Triclosan |
| L1300-13 | Ligand-26 | Carbenoxolone Sodium |
| L1300-13 | Ligand-88 | Danthron |
| L1300-13 | Ligand-22 | Bronopol |
| L1300-13 | Ligand-65 | Xylitol |
| L1300-13 | Ligand-25 | Terfenadine |
| L1300-13 | Ligand-77 | Cloxiquine |
| L1300-13 | Ligand-66 | Citiolone |
| L1300-13 | Ligand-32 | Trihexyphenidyl hydrochloride |
| L1300-13 | Ligand-33 | Bucetin |
| L1300-13 | Ligand-52 | DEET |
| L1300-13 | Ligand-64 | Methylene Blue |
| L1300-14 | Ligand-76 | Levofloxacin hydrate |
| L1300-14 | Ligand-69 | Parecoxib |
| L1300-14 | Ligand-71 | Tiagibine |
| L1300-14 | Ligand-77 | Dextromethorphan (hydrobromide hydrate) |
| L1300-14 | Ligand-68 | Ciclesonide |
| L1300-14 | Ligand-25 | Diiodohydroxyquinoline |
| L1300-14 | Ligand-38 | Resourcinol |
| L1300-14 | Ligand-28 | Benzyl alcohol |
| L1300-14 | Ligand-50 | Hydroquinone |
| L1300-14 | Ligand-64 | Gallic acid |
| L1300-14 | Ligand-65 | Nandrolone decanoate |
| L1300-14 | Ligand-3 | Succinylsulfathiazole |
| L1300-14 | Ligand-46 | Atazanavir |
| L1300-14 | Ligand-75 | Cefazolin Sodium |
| L1300-14 | Ligand-16 | Benzyl benzoate |
| L1300-14 | Ligand-20 | Lesinurad |
| L1300-14 | Ligand-11 | Lidocaine hydrochloride |
| L1300-14 | Ligand-51 | Chlormadinone acetate |
| L1300-14 | Ligand-10 | Nandrolone |
| L1300-14 | Ligand-30 | 5,5-Dimethyloxazolidine-2,4-dione |
| L1300-14 | Ligand-66 | (+/−)-Sulfinpyrazone |
| L1300-14 | Ligand-13 | Diethylcarbamazine (citrate) |
| L1300-14 | Ligand-21 | Atipamezole |
| L1300-14 | Ligand-31 | Isosorbide Mononitrate |
| L1300-14 | Ligand-52 | Acetohydroxamic acid |
| L1300-14 | Ligand-74 | Butamben |
| L1300-14 | Ligand-41 | Dapson |
| L1300-14 | Ligand-62 | Triacetin |
| L1300-14 | Ligand-2 | Sulfabenzamide |
| L1300-14 | Ligand-26 | Tyloxapol |
| L1300-14 | Ligand-59 | Hydroxyprogesterone caproate |
| L1300-14 | Ligand-61 | Halothane |
| L1300-14 | Ligand-44 | Zolpidem |
| L1300-14 | Ligand-40 | 5-Chloro-8-hydroxy-7-iodoquinoline |
| L1300-14 | Ligand-33 | Etoricoxib |
| L1300-14 | Ligand-42 | Alcaftadine |
| L1300-14 | Ligand-22 | Glycopyrrolate |
| L1300-14 | Ligand-27 | Amodiaquin (dihydrochloride dihydrate) |
| L1300-14 | Ligand-39 | Nitroxoline |
| L1300-14 | Ligand-45 | Sulisobenzone |
| L1300-14 | Ligand-70 | Molsidomine |
| L1300-14 | Ligand-19 | Hexachlorophene |
| L1300-14 | Ligand-8 | Brexpiprazole |
| L1300-14 | Ligand-9 | Atipamezole hydrochloride |
| L1300-14 | Ligand-47 | Etonogestrel |
| L1300-14 | Ligand-17 | Diflunisal |
| L1300-14 | Ligand-53 | Modafinil |
| L1300-14 | Ligand-4 | Lercanidipine (hydrochloride) |
| L1300-14 | Ligand-29 | Mebendazole |
| L1300-14 | Ligand-7 | Prochlorperazine dimaleate salt |
| L1300-14 | Ligand-49 | Fluphenazine (dihydrochloride) |
| L1300-14 | Ligand-73 | Hexylresorcinol |
| L1300-14 | Ligand-63 | Cephalothin |
| L1300-14 | Ligand-58 | Fusidate Sodium |
| L1300-14 | Ligand-37 | DL-Panthenol |
| L1300-14 | Ligand-57 | Sulpiride |
| L1300-14 | Ligand-6 | Itopride hydrochloride |
| L1300-14 | Ligand-35 | Benzocaine hydrochloride |
| L1300-14 | Ligand-54 | Ethosuximide |
| L1300-14 | Ligand-23 | Procaine |
| L1300-14 | Ligand-32 | Tedizolid Phosphate |
| L1300-14 | Ligand-56 | Armodafinil |
| L1300-14 | Ligand-14 | Terpin (hydrate) |
| L1300-14 | Ligand-5 | 2,2-Dihydroxy-4-methoxy benzophenone |
| L1300-14 | Ligand-78 | Chlorotrianisene |
| L1300-14 | Ligand-15 | Docusate Sodium |
| L1300-14 | Ligand-79 | Prasugrel Hydrochloride |
| L1300-14 | Ligand-83 | Gluconolactone |
| L1300-14 | Ligand-34 | Tiagabine hydrochloride |
| L1300-14 | Ligand-55 | Cyproheptadine hydrochloride |
| L1300-14 | Ligand-1 | Dehydrocholic acid |
| L1300-14 | Ligand-85 | Piperazine |
| L1300-14 | Ligand-43 | Sodium sulfadiazine |
| L1300-14 | Ligand-67 | Teneligliptin hydrobromide |
| L1300-14 | Ligand-86 | Butylparaben |
| L1300-14 | Ligand-88 | Folic acid |
| L1300-14 | Ligand-82 | Rebeprazole sodium |
| L1300-14 | Ligand-87 | Cefixime |
| L1300-14 | Ligand-81 | Eslicarbazepine Acetate |
| L1300-14 | Ligand-80 | Cefmenoxime hydrochloride |
| L1300-14 | Ligand-18 | Cefuroxime sodium |
| L1300-15 | Ligand-81 | Abemaciclib (LY2835219) |
| L1300-15 | Ligand-60 | Selinexor (KPT-330) |
| L1300-15 | Ligand-24 | Escin |
| L1300-15 | Ligand-18 | Ribociclib (LEE011) |
| L1300-15 | Ligand-35 | Oxybenzone |
| L1300-15 | Ligand-45 | Sildenafil Mesylate |
| L1300-15 | Ligand-26 | Certinib (LDK378) |
| L1300-15 | Ligand-55 | Entrectinib (RXDX-101) |
| L1300-15 | Ligand-79 | Saxagliptin hydrate |
| L1300-15 | Ligand-34 | Loxoprofen |
| L1300-15 | Ligand-16 | BAF312 (Siponimod) |
| L1300-15 | Ligand-46 | Guanfacine Hydrochloride |
| L1300-15 | Ligand-58 | Birinapant |
| L1300-15 | Ligand-68 | ArbinoxaMine Maleate |
| L1300-15 | Ligand-57 | D panthenol |
| L1300-15 | Ligand-86 | Docetaxel Trihydrate |
| L1300-15 | Ligand-25 | Tacrolimus (FK506) |
| L1300-15 | Ligand-49 | Alvelestat (AZD9668) |
| L1300-15 | Ligand-82 | Suvorexant (MK-4305) |
| L1300-15 | Ligand-78 | Rivastigmine |
| L1300-15 | Ligand-67 | Vitamin E |
| L1300-15 | Ligand-59 | Marimastat (BB-2516) |
| L1300-15 | Ligand-5 | Motolimod (VTX-2337) |
| L1300-15 | Ligand-6 | Edoxaban |
| L1300-15 | Ligand-12 | Terazosin |
| L1300-15 | Ligand-13 | Deoxycholic acid |
| L1300-15 | Ligand-37 | Tzemetostat (EPZ-6438) |
| L1300-15 | Ligand-17 | Rociletinib (CO-1686) (AVL-301) |
| L1300-15 | Ligand-80 | IPI-145 (INK1197) |
| L1300-15 | Ligand-72 | Aloxistatin |
| L1300-15 | Ligand-84 | Filgotinib (GLPG0634) |
| L1300-15 | Ligand-15 | Cilengitide |
| L1300-15 | Ligand-3 | Tofacitinib (CP-690550) Citrate |
| L1300-15 | Ligand-1 | Povidone iodine |
| L1300-15 | Ligand-88 | Empagliflozin (BI10773) |
| L1300-15 | Ligand-70 | Ilomastat (GM6001, Galardin) |
| L1300-15 | Ligand-77 | Otenabant (CP-945598) HCl |
| L1300-15 | Ligand-85 | Sacubitril/Valsartan LCZ696 |
| L1300-15 | Ligand-4 | Tepotinib (EMD1214063) |
| L1300-15 | Ligand-47 | Binimetinib MEK162 (ARRY-162, |
| | | ARRY-438162) |
| L1300-15 | Ligand-48 | Batimastat (BB-94) |
| L1300-15 | Ligand-2 | Perampanel |
| L1300-15 | Ligand-75 | Erlotinib |
| L1300-15 | Ligand-42 | Emricasan |
| L1300-15 | Ligand-38 | Losmapimod (GW856553X) |
| L1300-15 | Ligand-76 | Tenofovir Alafenamide (GS-7340) |
| L1300-15 | Ligand-56 | Efavirenz |
| L1300-15 | Ligand-8 | Lomitapide Mesylate |
| L1300-15 | Ligand-41 | Pilaralisib (XL147) |
| L1300-15 | Ligand-23 | Protirelin |
| L1300-15 | Ligand-7 | Puromycin 2HCl |
| L1300-15 | Ligand-52 | Voxtalisib (XL765, SAR245409) |
| L1300-15 | Ligand-63 | Defactinib (VS-6063, PF-04554878) |
| L1300-15 | Ligand-29 | Uprosertib (GSK2141795) |
| L1300-15 | Ligand-21 | Afatinib (BIBW2992) Dimaleate |
| L1300-15 | Ligand-40 | Trelagliptin |
| L1300-15 | Ligand-87 | Oltipraz |
| L1300-15 | Ligand-53 | Sunitinib |
| L1300-15 | Ligand-54 | Eliglustat Tartrate |
| L1300-15 | Ligand-71 | Vidofludimus |
| L1300-15 | Ligand-62 | Entospletinib (GS-9973) |
| L1300-15 | Ligand-28 | Osimertinib (AZD9291) |
| L1300-15 | Ligand-73 | Ledipasvir (GS5885) |
| L1300-15 | Ligand-33 | Napabucasin |
| L1300-15 | Ligand-66 | Rocilinostat (ACY-1215) |
| L1300-15 | Ligand-50 | Tasisulam |
| L1300-15 | Ligand-43 | Tretazicar (CB1954) |
| L1300-15 | Ligand-30 | Lomitapide |
| L1300-15 | Ligand-39 | Rilpivirine |
| L1300-15 | Ligand-51 | Afuresertib (GSK2110183) |
| L1300-15 | Ligand-36 | Pimecrolimus |
| L1300-15 | Ligand-20 | Dovitinib (TKI258) Lactate |
| L1300-15 | Ligand-11 | Epacadostat (INCB024360) |
| L1300-15 | Ligand-22 | Ozanimod (RPC1063) |
| L1300-15 | Ligand-9 | Salirasib |
| L1300-15 | Ligand-14 | Fingolimod (FTY720) HCl |
| L1300-15 | Ligand-64 | Dasatinib Monohydrate |
| L1300-15 | Ligand-61 | Poziotinib (HM781-36B) |
| L1300-15 | Ligand-31 | Elacridar (GF120918) |
| L1300-15 | Ligand-65 | Ulixertinib (BVD-523, VRT752271) |
| L1300-15 | Ligand-44 | Ripasudil (K-115) |
| L1300-15 | Ligand-10 | Pexmetinib (ARRY-614) |
| L1300-15 | Ligand-32 | Pexidartinib (PLX3397) |
| L1300-15 | Ligand-74 | Obeticholic Acid |
| L1300-15 | Ligand-19 | Cerdulatinib (PRT062070, PRT2070) |
| L1300-15 | Ligand-27 | Idasanutlin (RG-7388) |
| L1300-15 | Ligand-69 | Embelin |
| L1300-15 | Ligand-83 | Sorafenib |
| L1300-16 | Ligand-40 | Daptomycin |
| L1300-16 | Ligand-73 | Polymyxin B sulphate |
| L1300-16 | Ligand-49 | Atosiban Acetate |
| L1300-16 | Ligand-10 | Sodium butyrate |
| L1300-16 | Ligand-2 | Radotinib |
| L1300-16 | Ligand-13 | Riociguat (BAY 63-2521) |
| L1300-16 | Ligand-60 | Oxytocin (Syntocinon) |
| L1300-16 | Ligand-30 | Palbociclib (PD0332991) Isethionate |
| L1300-16 | Ligand-20 | D-glutamine |
| L1300-16 | Ligand-24 | Molidustat (BAY 85-3934) |
| L1300-16 | Ligand-39 | Gemcitabine HCl |
| L1300-16 | Ligand-57 | Oclacitinib |
| L1300-16 | Ligand-71 | Salmon Calcitonin Acetate |
| L1300-16 | Ligand-35 | Empty |
| L1300-16 | Ligand-12 | Cobimetinib (GDC-0973, RG7420) |
| L1300-16 | Ligand-67 | Bardoxolone Methyl |
| L1300-16 | Ligand-76 | Gabapentin |
| L1300-16 | Ligand-88 | Streptomycin sulfate |
| L1300-16 | Ligand-31 | Disodium Cromoglycate |
| L1300-16 | Ligand-9 | Tranexamic Acid |
| L1300-16 | Ligand-62 | Mizoribine |
| L1300-16 | Ligand-34 | Empty |
| L1300-16 | Ligand-65 | Fudosteine |
| L1300-16 | Ligand-50 | Carboplatin |
| L1300-16 | Ligand-38 | Octreotide Acetate |
| L1300-16 | Ligand-18 | Heparin sodium |
| L1300-16 | Ligand-53 | Metformin HCl |
| L1300-16 | Ligand-72 | Gabapentin HCl |
| L1300-16 | Ligand-41 | Cytarabine |
| L1300-16 | Ligand-28 | Pemetrexed |
| L1300-16 | Ligand-1 | Apremilast (CC-10004) |
| L1300-16 | Ligand-82 | GHRP-2 |
| L1300-16 | Ligand-19 | Perindopril Erbumine |
| L1300-16 | Ligand-21 | Taurine |
| L1300-16 | Ligand-83 | Galanthamine HBr |
| L1300-16 | Ligand-6 | Nafarelin Acetate |
| L1300-16 | Ligand-75 | Procarbazine HCl |
| L1300-16 | Ligand-11 | Salbutamol Sulfate |
| L1300-16 | Ligand-29 | Biapenem |
| L1300-16 | Ligand-45 | Pacritinib (SB1518) |
| L1300-16 | Ligand-43 | Lisinopril |
| L1300-16 | Ligand-63 | Nedaplatin |
| L1300-16 | Ligand-61 | Pamidronate Disodium |
| L1300-16 | Ligand-42 | (−)-Tetramisole |
| L1300-16 | Ligand-51 | Dorzolamide HCl |
| L1300-16 | Ligand-77 | Neomycin sulfate |
| L1300-16 | Ligand-64 | Ticlopidine HCl |
| L1300-16 | Ligand-44 | Lomefloxacin HCl |
| L1300-16 | Ligand-85 | Etidronate |
| L1300-16 | Ligand-16 | Eptifibatide Acetate |
| L1300-16 | Ligand-74 | Penicillamine |
| L1300-16 | Ligand-17 | Palbociclib (PD-0332991) HCl |
| L1300-16 | Ligand-66 | Oxacillin sodium monohydrate |
| L1300-16 | Ligand-55 | Hydralazine HCl |
| L1300-16 | Ligand-78 | Sotagliflozin (LX4211) |
| L1300-16 | Ligand-87 | Donepezil HCl |
| L1300-16 | Ligand-54 | Fosinopril Sodium |
| L1300-16 | Ligand-22 | Tobramycin |
| L1300-16 | Ligand-56 | Vorapaxar |
| L1300-16 | Ligand-32 | Clindamycin Phosphate |
| L1300-16 | Ligand-5 | Bivalirudin Trifluoroacetate |
| L1300-16 | Ligand-52 | L-Glutamine |
| L1300-16 | Ligand-8 | Cephalexin |
| L1300-16 | Ligand-84 | LY2228820 |
| L1300-16 | Ligand-46 | Pimavanserin |
| L1300-16 | Ligand-86 | D-Cycloserine |
| L1300-16 | Ligand-68 | Bitopertin |
| L1300-16 | Ligand-7 | Granisetron HCl |
| L1300-16 | Ligand-27 | Lypressin Acetate |
| L1300-16 | Ligand-23 | Venetoclax ABT-199 (GDC-0199) |
| L1300-16 | Ligand-79 | Ponesimod |
| L1300-16 | Ligand-33 | Methacycline HCl |
| L1300-16 | Ligand-3 | Olmutinib (HM61713, BI1482694) |
| L1300-17 | Ligand-40 | Empty |
| L1300-17 | Ligand-67 | Miltefosine |
| L1300-17 | Ligand-7 | Aprotinin |
| L1300-17 | Ligand-14 | Colistin Sulfate |
| L1300-17 | Ligand-84 | Empty |
| L1300-17 | Ligand-50 | L-Ornithine |
| L1300-17 | Ligand-29 | Empty |
| L1300-17 | Ligand-28 | Eflornithine hydrochloride hydrate |
| L1300-17 | Ligand-70 | Proflavine Hemisulfate |
| L1300-17 | Ligand-49 | Hydroxychloroquine Sulfate |
| L1300-17 | Ligand-82 | Antimonyl (potassium tartrate trihydrate) |
| L1300-17 | Ligand-51 | Empty |
| L1300-17 | Ligand-36 | Netilmicin Sulfate |
| L1300-17 | Ligand-12 | Calcium Levofolinate |
| L1300-17 | Ligand-4 | Chloroquine Phosphate |
| L1300-17 | Ligand-18 | Pemetrexed Disodium Hydrate |
| L1300-17 | Ligand-61 | Cefradine |
| L1300-17 | Ligand-2 | Amikacin disulfate |
| L1300-17 | Ligand-35 | Tripelennamine HCl |
| L1300-17 | Ligand-16 | Calcium Gluceptate |
| L1300-17 | Ligand-3 | Flavoxate HCl |
| L1300-17 | Ligand-39 | Glutathione |
| L1300-17 | Ligand-13 | (R)-baclofen |
| L1300-17 | Ligand-83 | Fosbretabulin (Combretastatin A4 |
| | | Phosphate (CA4P)) Disodium |
| L1300-17 | Ligand-60 | Dihydrostreptomycin sulfate |
| L1300-17 | Ligand-25 | Gentamicin Sulfate |
| L1300-17 | Ligand-73 | Empty |
| L1300-17 | Ligand-34 | Zanamivir |
| L1300-17 | Ligand-17 | Pralidoxime (chloride) |
| L1300-17 | Ligand-46 | Ibandronate sodium |
| L1300-17 | Ligand-47 | Hexamethonium Bromide |
| L1300-17 | Ligand-72 | Sildenafil |
| L1300-17 | Ligand-56 | Palonosetron HCl |
| L1300-17 | Ligand-80 | Amprolium HCl |
| L1300-17 | Ligand-38 | Tolmetin Sodium |
| L1300-17 | Ligand-68 | L-Arginine HCl (L-Arg) |
| L1300-17 | Ligand-15 | Ceftriaxone Sodium Trihydrate |
| L1300-17 | Ligand-1 | Vancomycin HCl |
| L1300-17 | Ligand-62 | Empty |
| L1300-17 | Ligand-59 | Capreomycin Sulfate |
| L1300-17 | Ligand-45 | Solifenacin succinate |
| L1300-17 | Ligand-57 | Abacavir sulfate |
| L1300-17 | Ligand-79 | Pemirolast potassium |
| L1300-17 | Ligand-81 | Sodium ascorbate |
| L1300-17 | Ligand-6 | Eprodisate (disodium) |
| L1300-17 | Ligand-23 | Hygromycin B |
| L1300-17 | Ligand-78 | Danofloxacin Mesylate |
| L1300-17 | Ligand-71 | Sisomicin sulfate |
| L1300-17 | Ligand-37 | Paromomycin Sulfate |
| L1300-17 | Ligand-27 | Ceftazidime Pentahydrate |
| L1300-17 | Ligand-48 | Minocycline HCl |
| L1300-17 | Ligand-5 | Amifostine |
| L1300-17 | Ligand-58 | Tetramisole HCl |
| L1300-17 | Ligand-69 | Terbutaline Sulfate |
| L1300-17 | Ligand-24 | Amikacin hydrate |
| L1300-17 | Ligand-26 | Nefopam HCl |

### Method of Hit Validation

Dose-Response Validation: 25 hits were further analyzed using the same method as the screening except at 16 different drug concentrations. Drugs showing Michaelis-Menten-like dose response were recorded, and dissociation constants were determined through curve fitting.

A summary of selected hits identified in the screening along with their measured dissociation constants, if available is shown below:

| Statistical | | | | | | | | |
|---|---|---|---|---|---|---|---|---|
| significance | Δfnorm | | | | Tested | | | |
| in the | in the | | Plate | Well | for dose | S/N > | | |
| screening | screening | Compound name | number | number | response? | 5? | Kd | |
| **** | ++++ | Foretinib | L1300-1 | 39 | Y | N | N/A | |
| | | (GSK1363089) | | | | | | |
| ** | −− | 17-AAG | L1300-1 | 40 | Y | N | N/A | |
| | | (Tanespimycin) | | | | | | |
| **** | −− | Nilotinib (AMN-107) | L1300-1 | 47 | Y | Y | 16.1 | μM |
| ** | −− | VX-680 (Tozasertib, | L1300-1 | 48 | Y | N | N/A | |
| | | MK-0457) | | | | | | |
| ** | ++ | Ivacaftor (VX-770) | L1300-1 | 51 | Y | Y | 18.4 | μM |
| *** | −−− | OSI-906 (Linsitinib) | L1300-1 | 82 | Y | N | N/A | |
| *** | ++ | Epirubicin HCl | L1300-2 | 56 | Y | N | N/A | |
| **** | +++ | Zosuquidar | L1300-3 | 50 | Y | N | N/A | |
| | | (LY335979) 3HCl | | | | | | |
| ** | ++ | Ziprasidone HCl | L1300-3 | 59 | Y | N | N/A | |
| ***** | −− | Acetylcysteine | L1300-4 | 46 | Y | N | N/A | |
| ***** | −− | Zafirlukast | L1300-4 | 47 | Y | Y | 2.52 | μM |
| ** | ++ | Elvitegravir (GS-9137, | L1300-6 | 7 | Y | N | N/A | |
| | | JTK-303) | | | | | | |
| ** | ++ | Meclizine 2HCl | L1300-6 | 39 | N | N/A | N/A | |
| *** | ++ | Candesartan Cilexetil | L1300-6 | 42 | Y | Y | 27.8 | nm |
| ** | +++ | Diclazuril | L1300-6 | 52 | Y | Y | 77.9 | nm |
| ***** | +++ | Flunarizine 2HCl | L1300-6 | 74 | Y | N | N/A | |
| ** | +++++ | Evacetrapib | L1300-9 | 18 | Y | N | N/A | |
| | | (LY2484595) | | | | | | |
| ** | −−− | Estradiol valerate | L1300-10 | 59 | Y | Y | 6.84 | nm |
| ** | ++ | Etravirine (TMC125) | L1300-10 | 78 | Y | Y | 7.88 | μM |
| ***** | ++ | Montelukast Sodium | L1300-12 | 27 | Y | N | N/A | |
| ***** | ++ | Nelfinavir Mesylate | L1300-12 | 86 | Y | N | N/A | |
| ***** | −−− | Mefloquine HCl | L1300-13 | 7 | Y | N | N/A | |
| *** | ++ | Pimozide | L1300-13 | 58 | Y | Y | 29.3 | nm |

| | | | | | | | |
|---|---|---|---|---|---|---|---|
| *** | −−− | Zolpidem | L1300-15 | 41 | Y | N | N/A |
| ***** | −−−− | Hydroxyprogesterone | L1300-15 | 55 | N | N/A | N/A |

caproate

(*: p < 0.05, **: p < 0.01, ***: p < 0.001, ****: p < 0.0001, *****: p < 0.00001) (+: Δfnorm > 20‰, ++: Δfnorm > 40‰, +++: Δfnorm > 60‰, ++++: Δfnorm > 80‰, ++ +++: Δfnorm > 100‰, −: Δfnorm < −20‰, −−: Δfnorm < −40‰, −−−: Δfnorm < −60‰, −−−−: Δfnorm < −80‰, −−−−−: Δfnorm < −100‰)

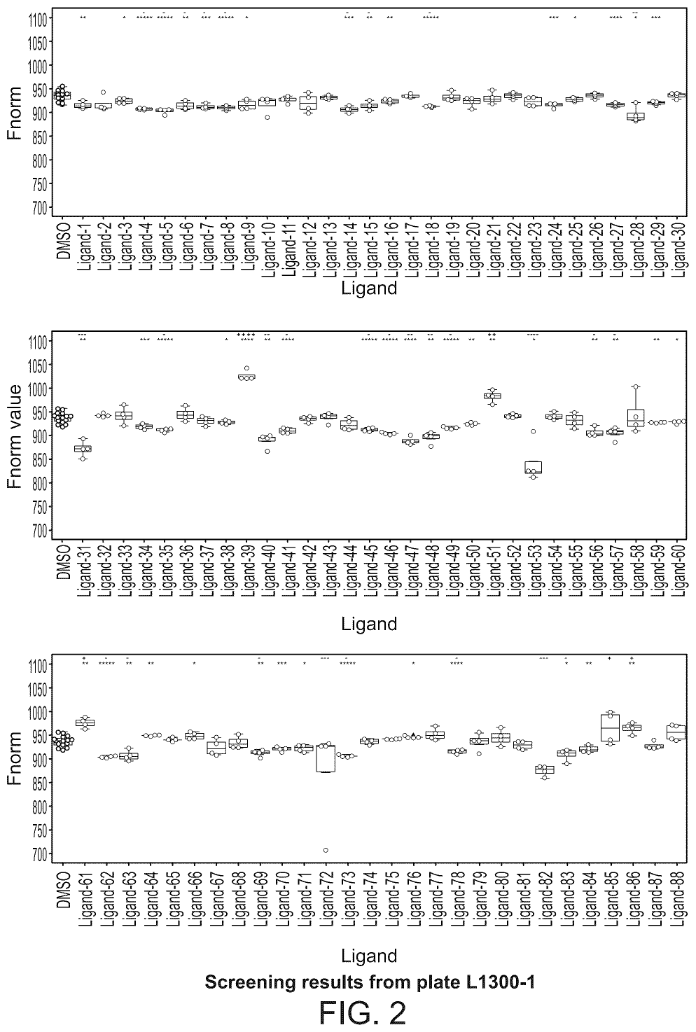

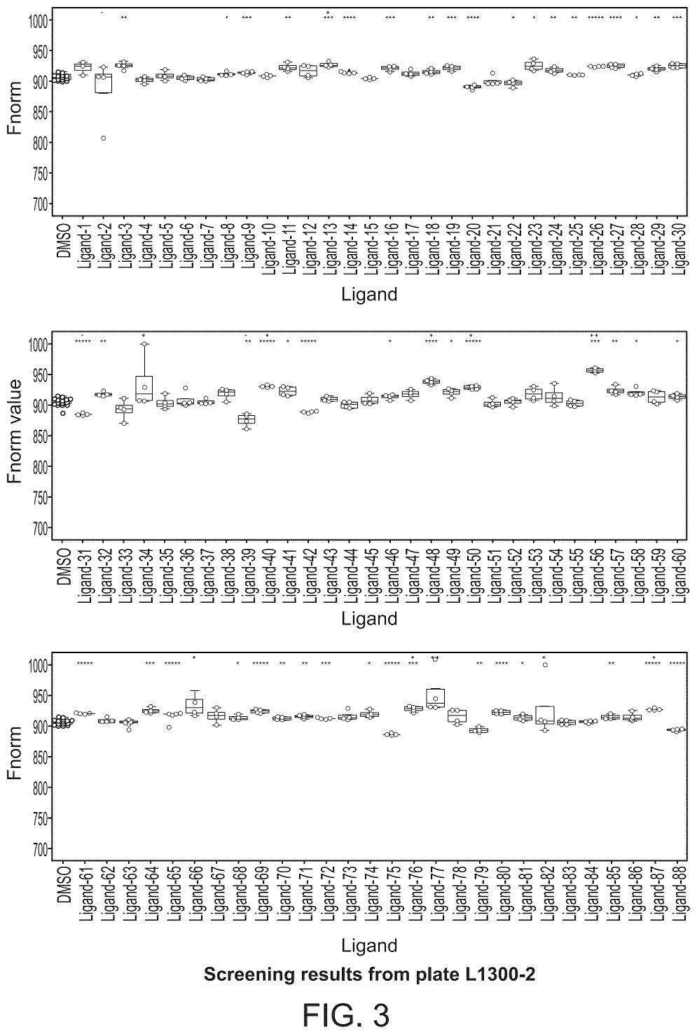

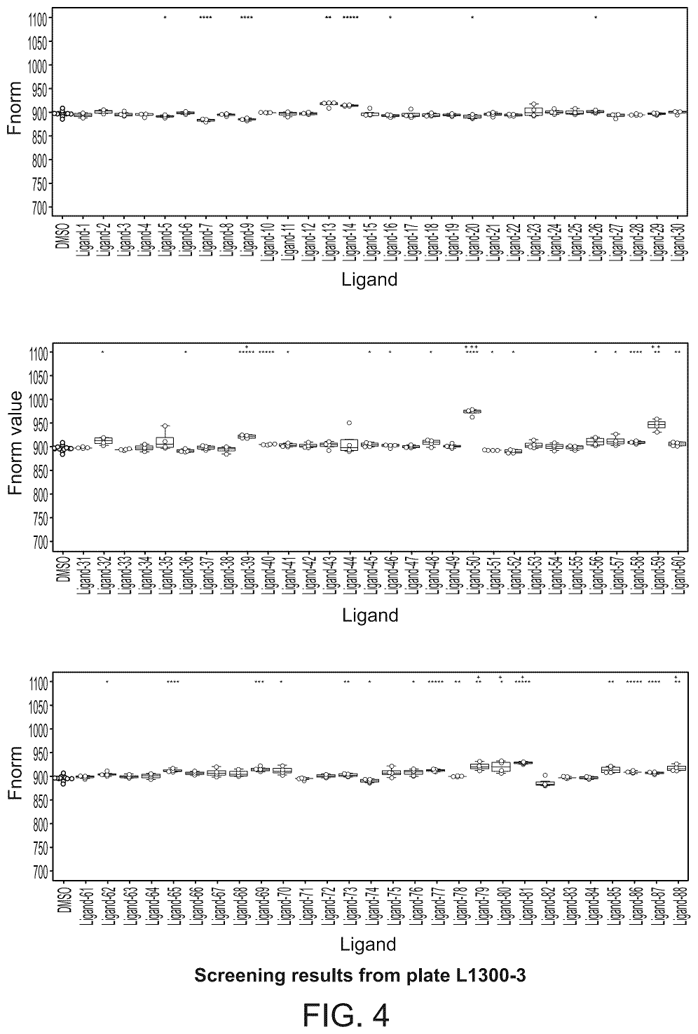

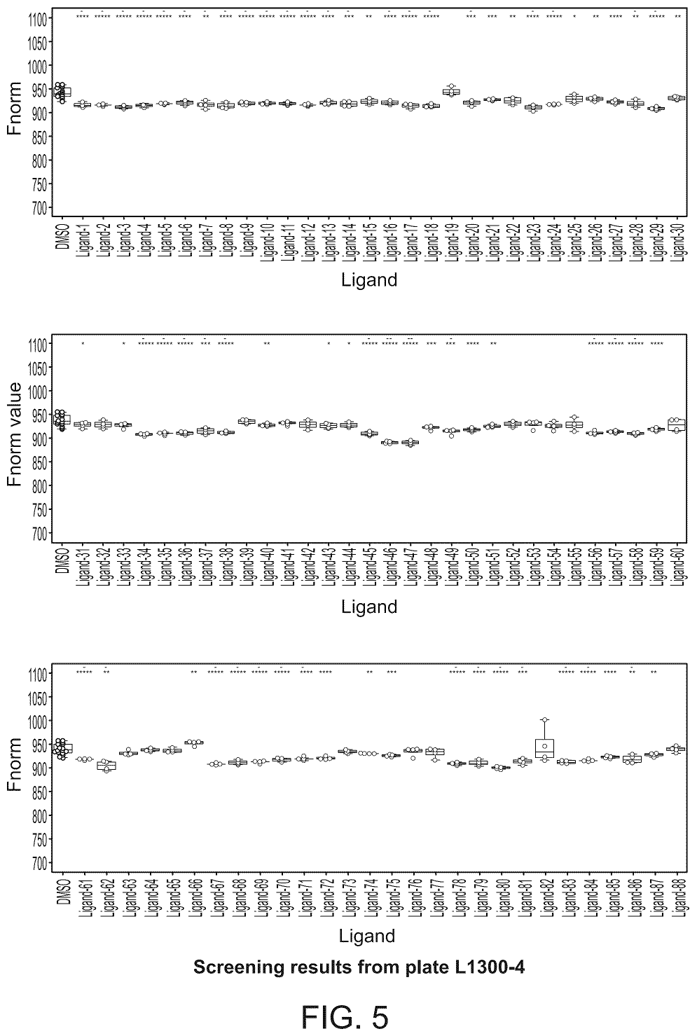

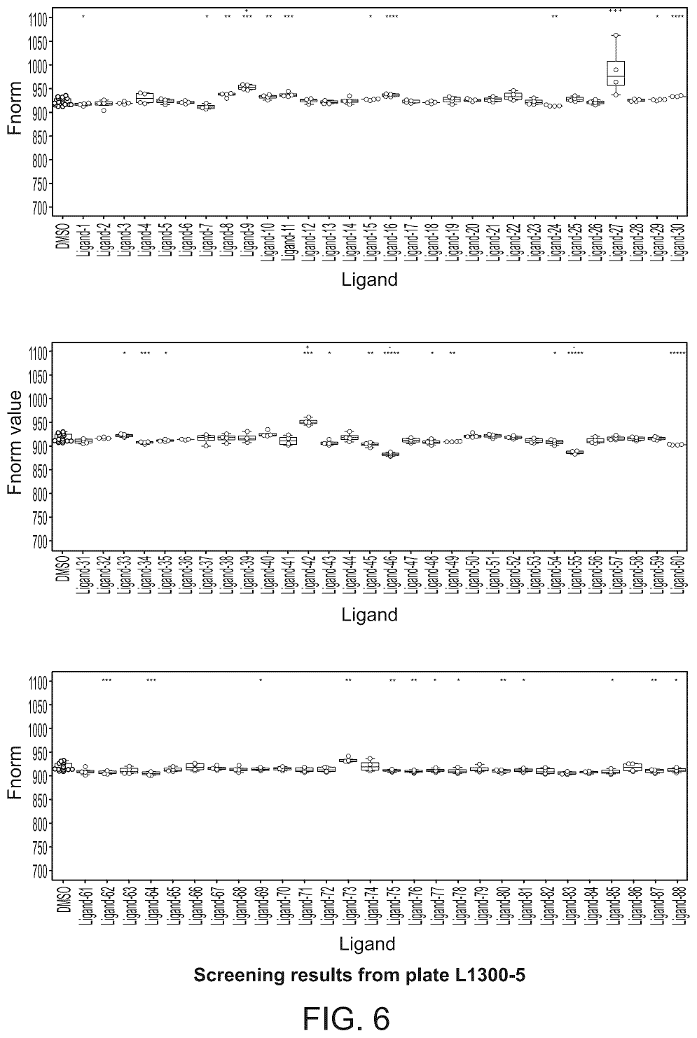

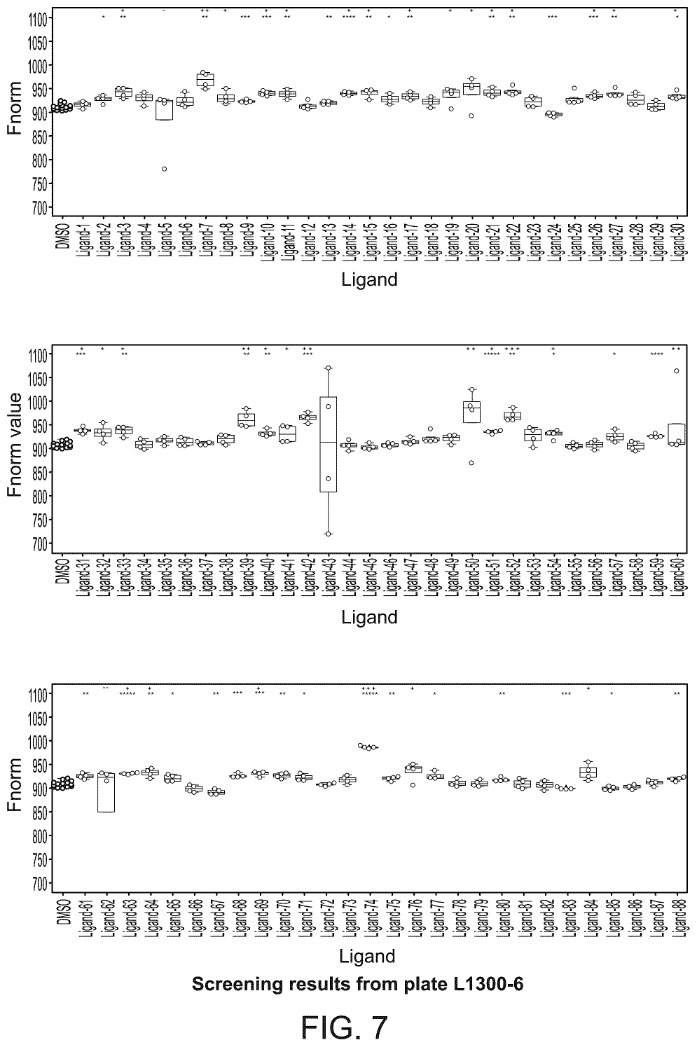

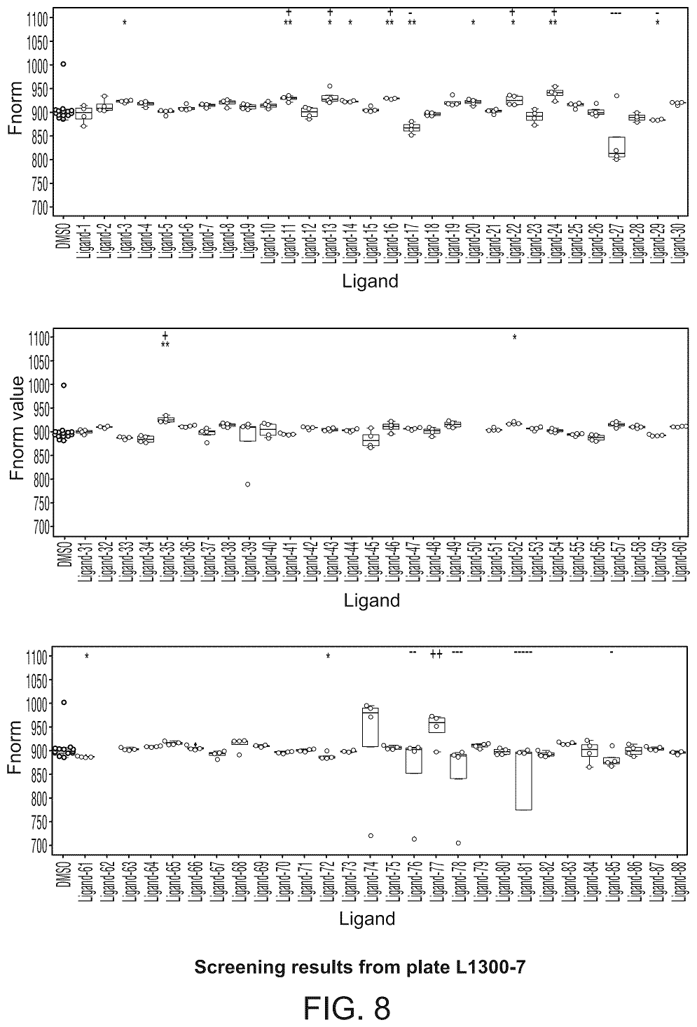

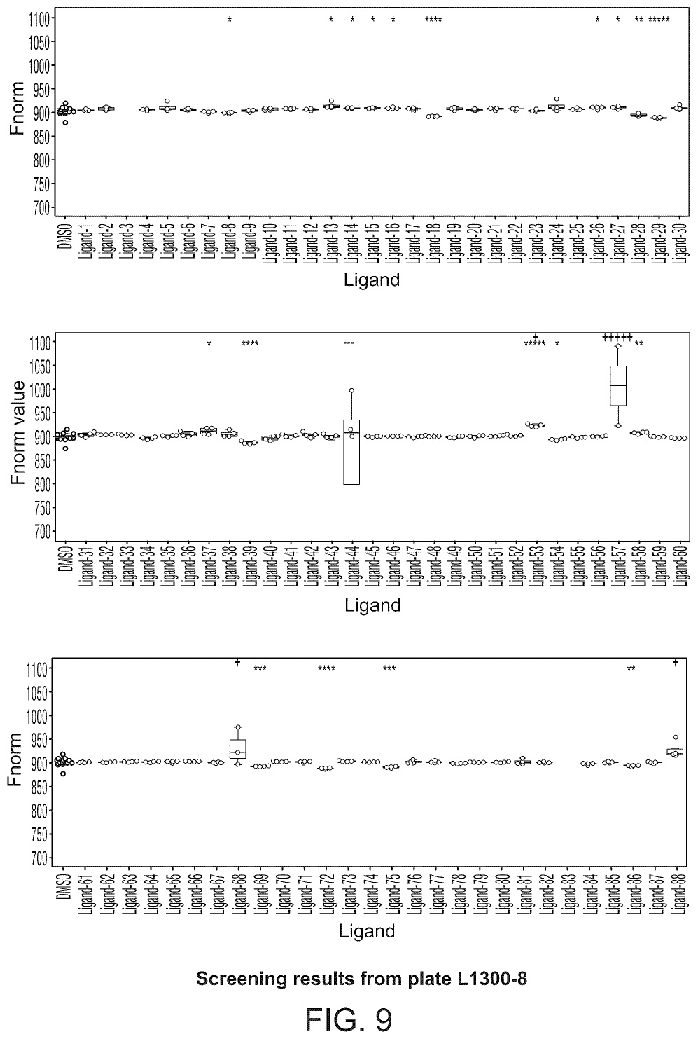

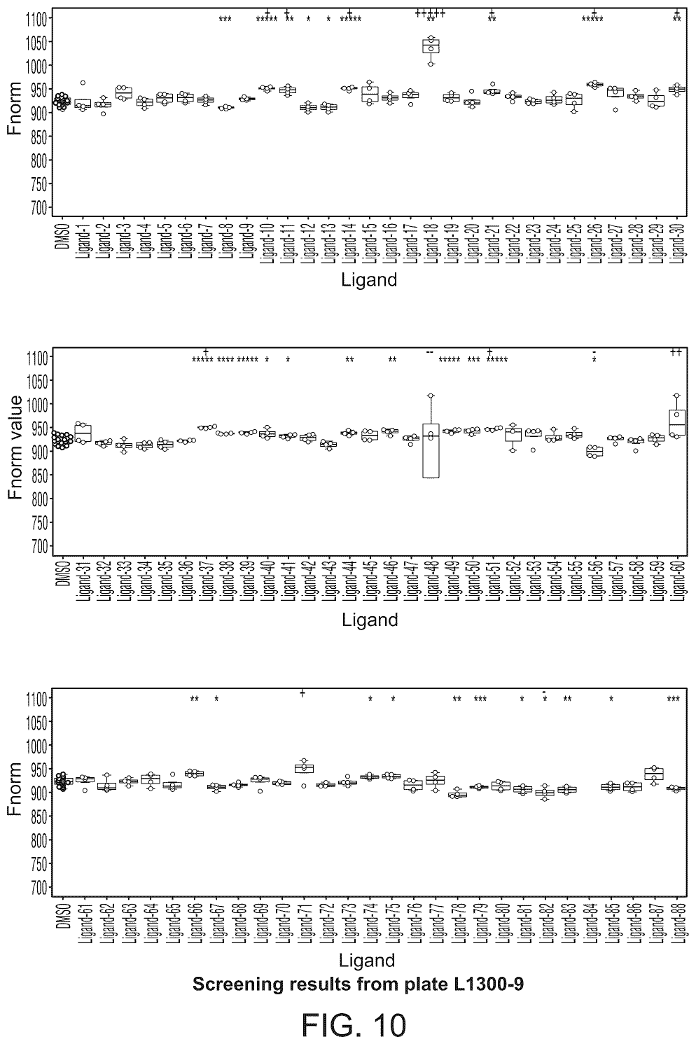

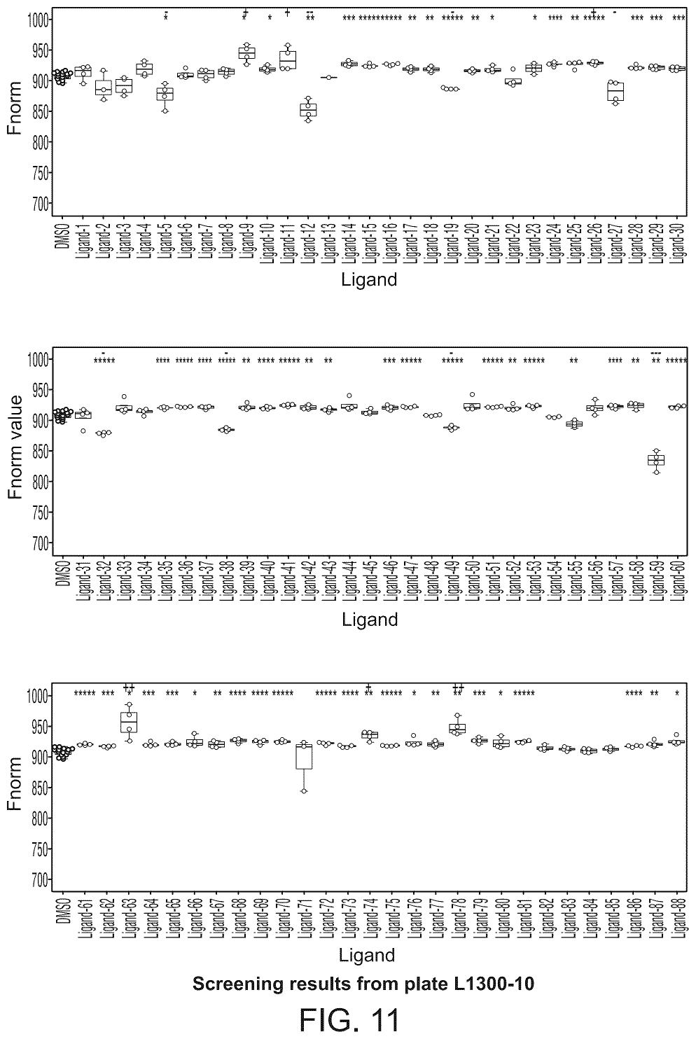

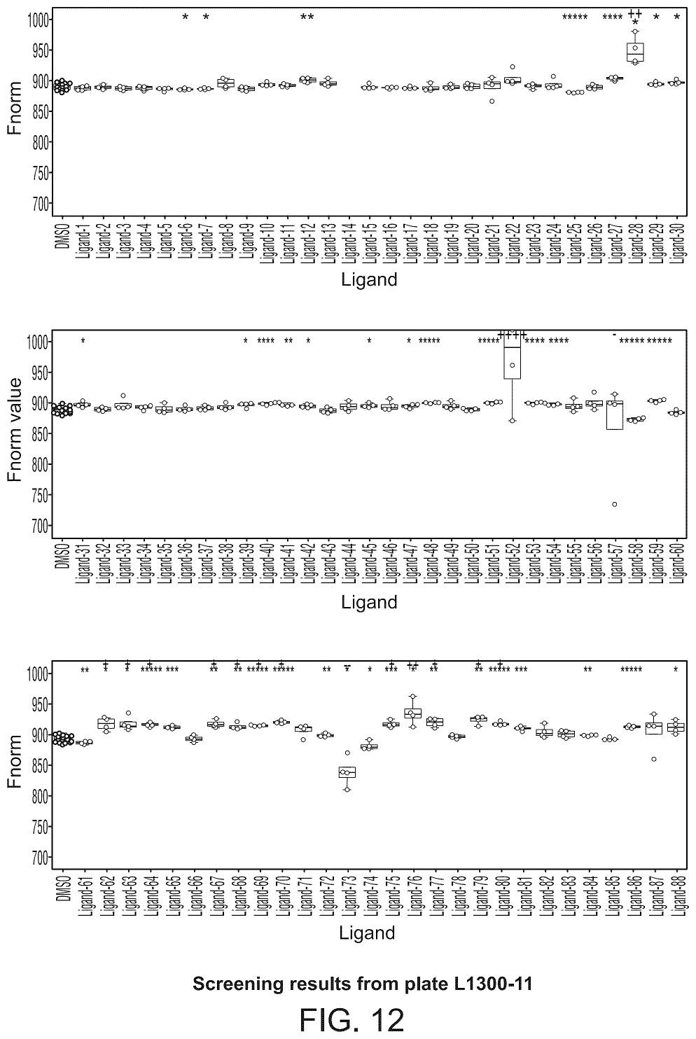

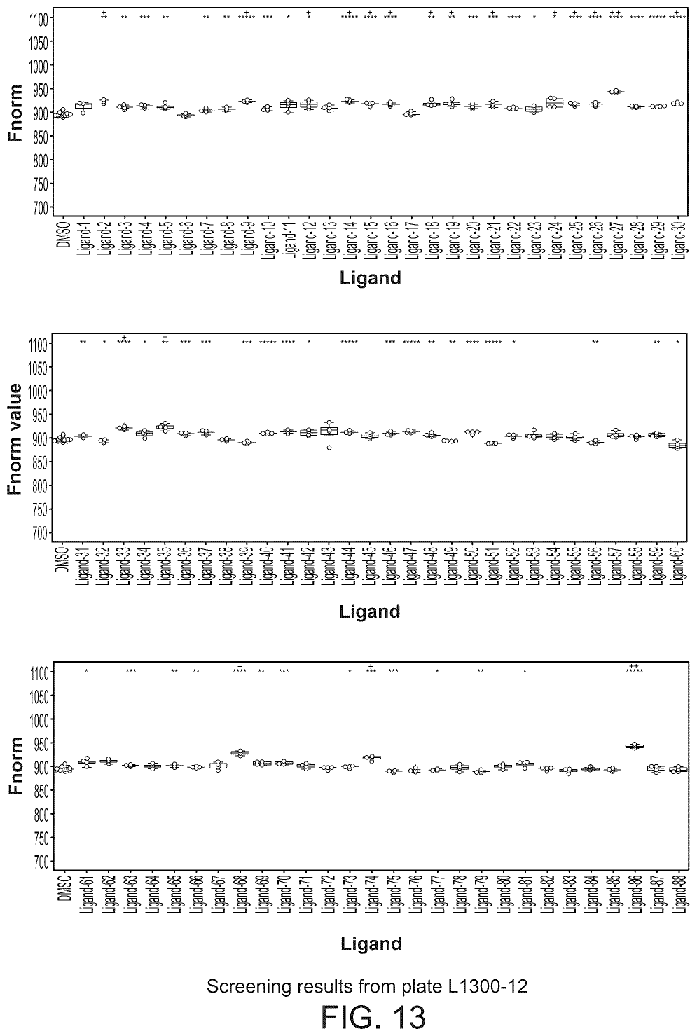

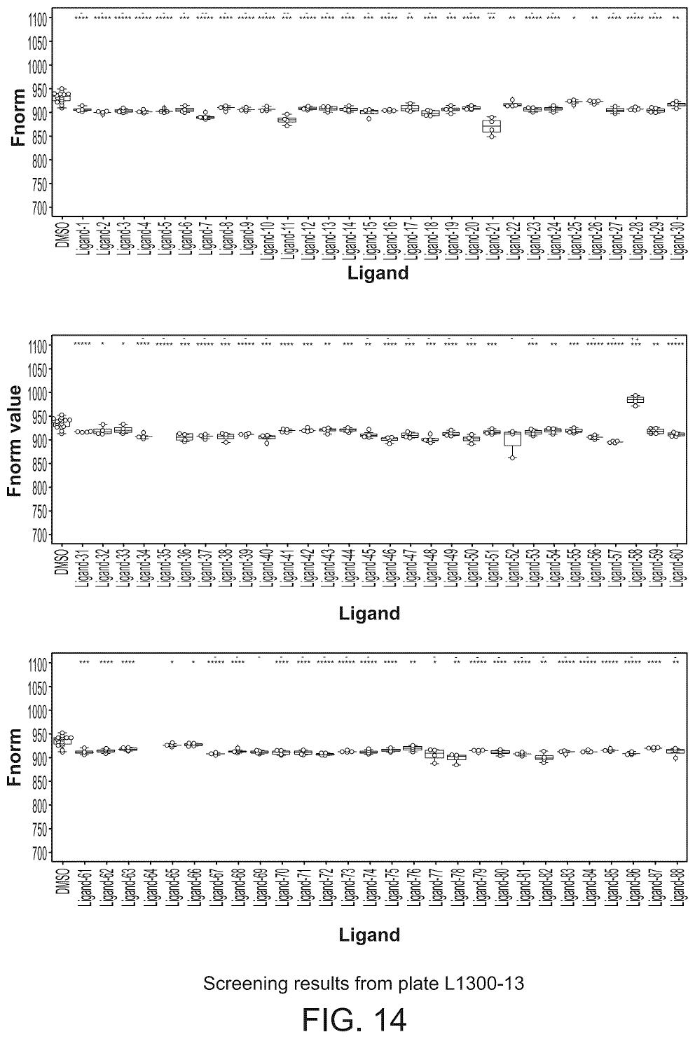

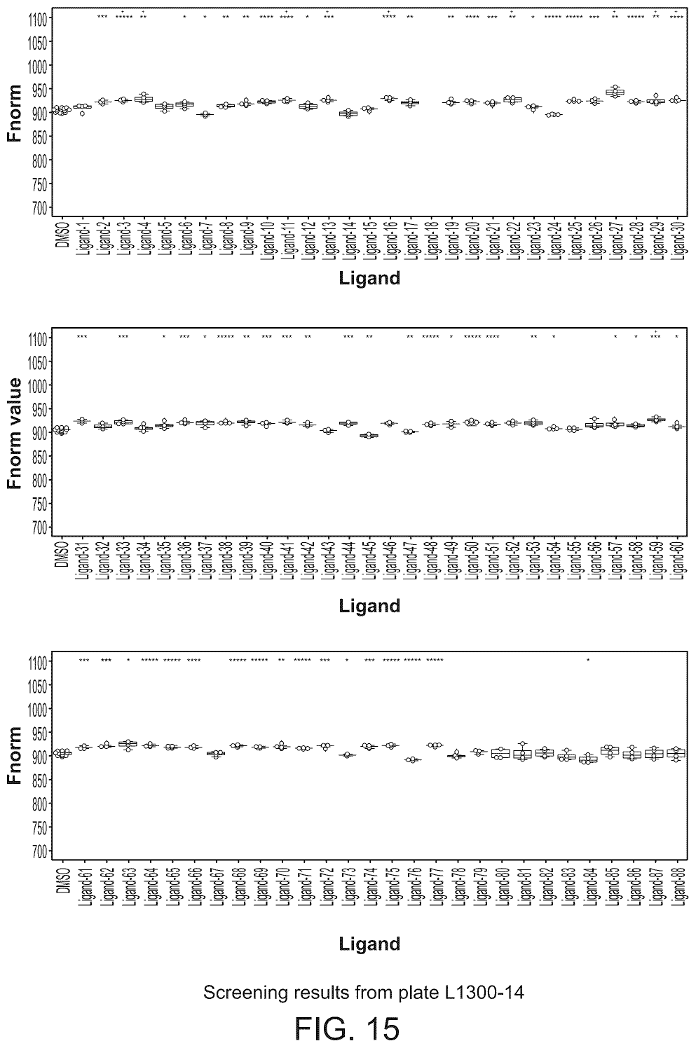

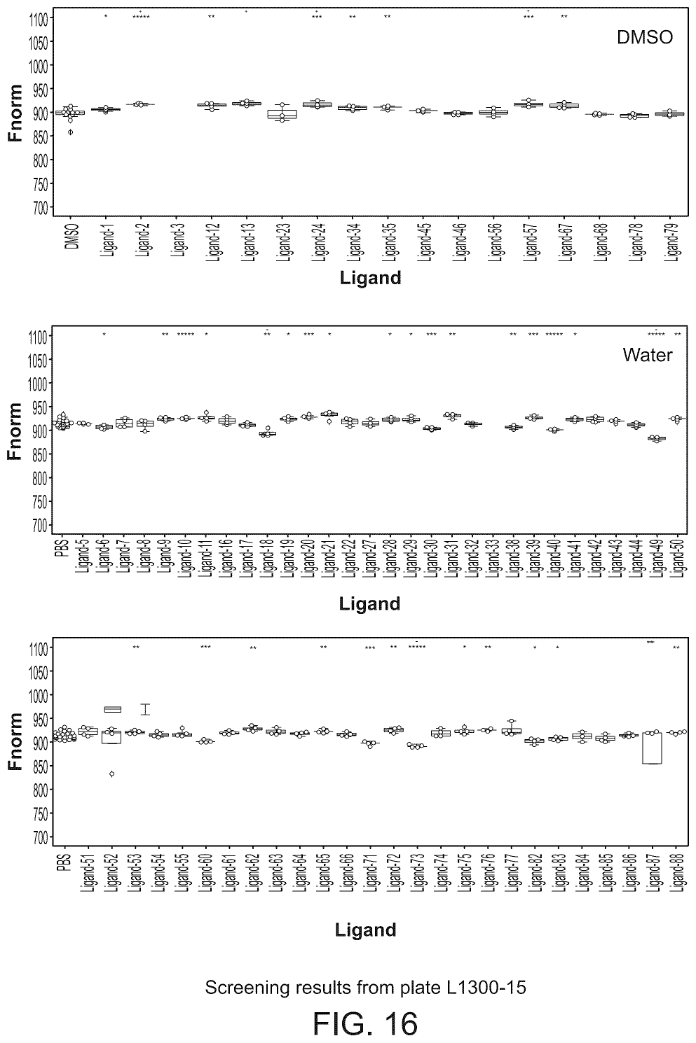

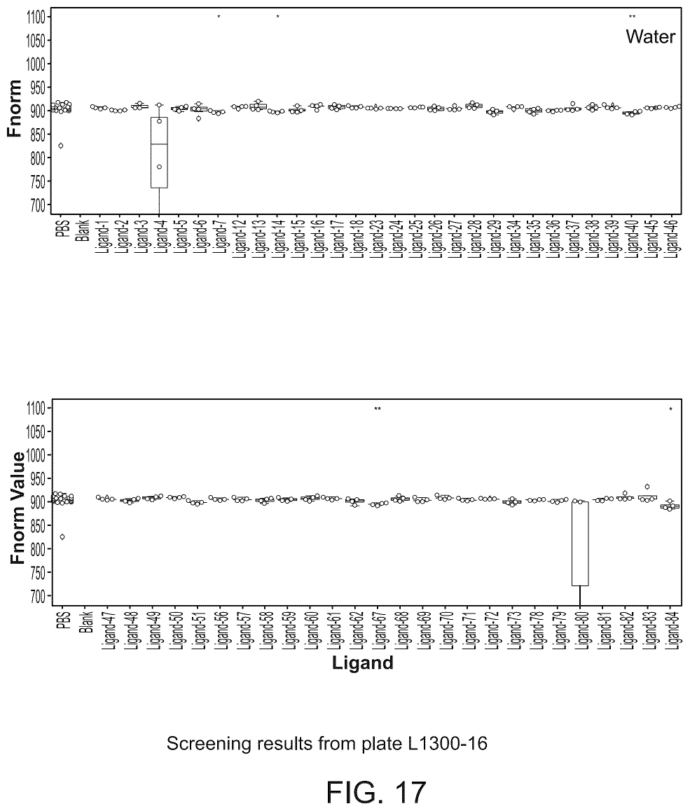

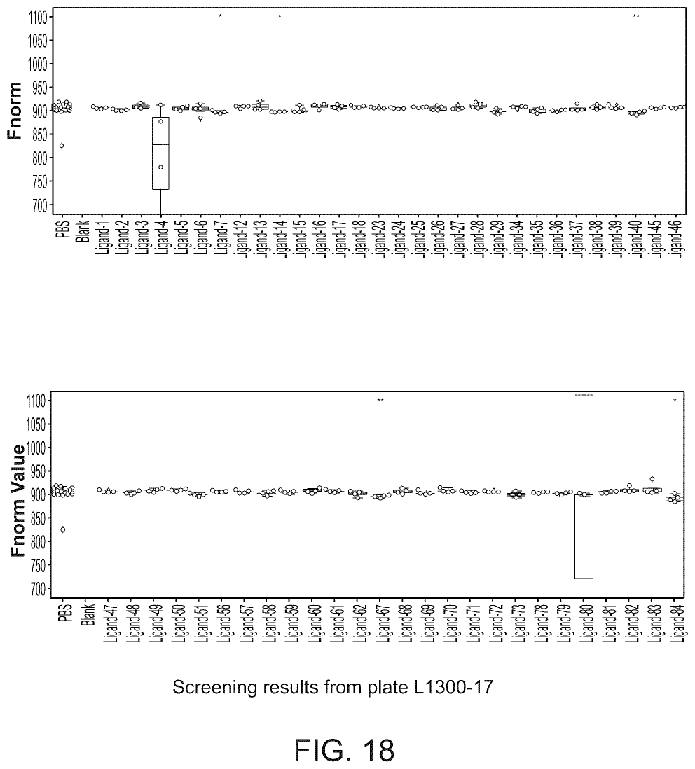

FIG. 2-18 are graphs of plate-by-plate screening results.

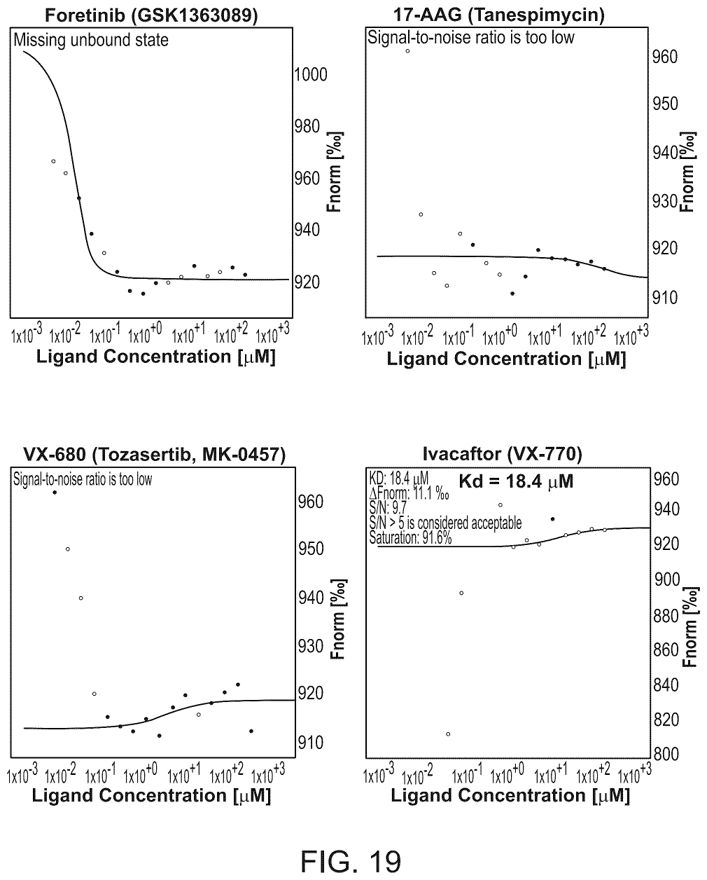

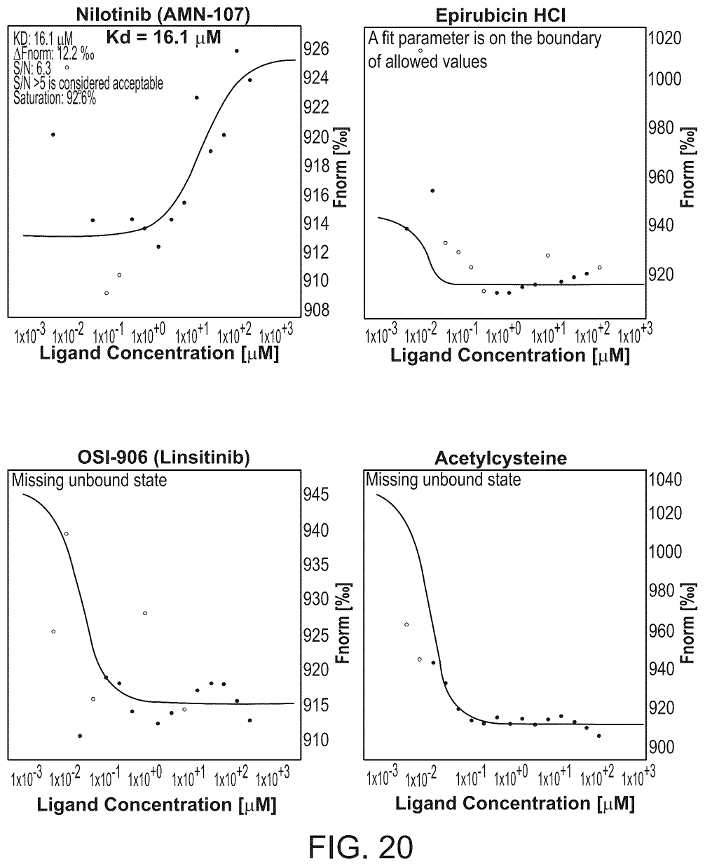

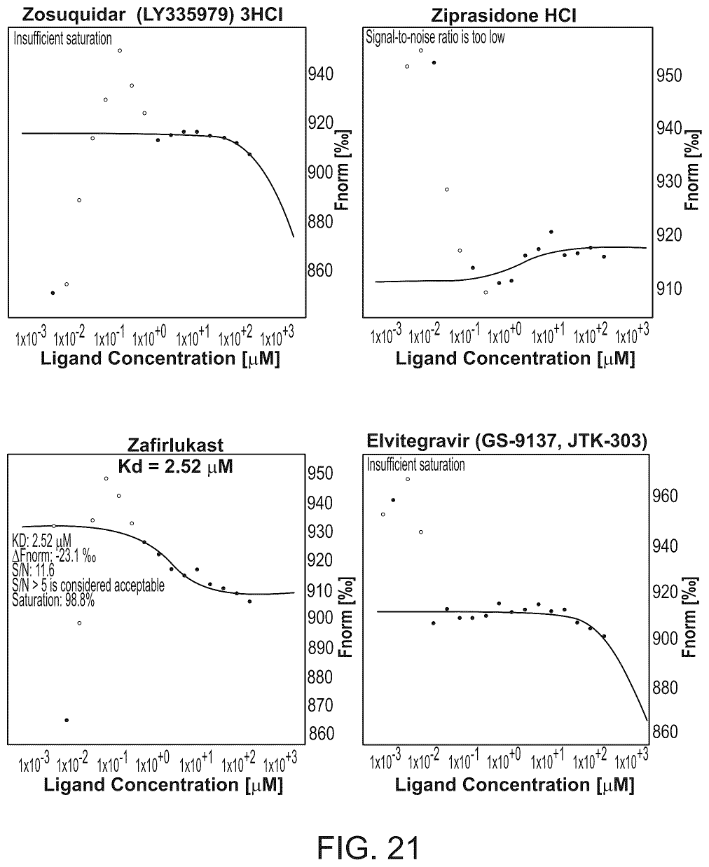

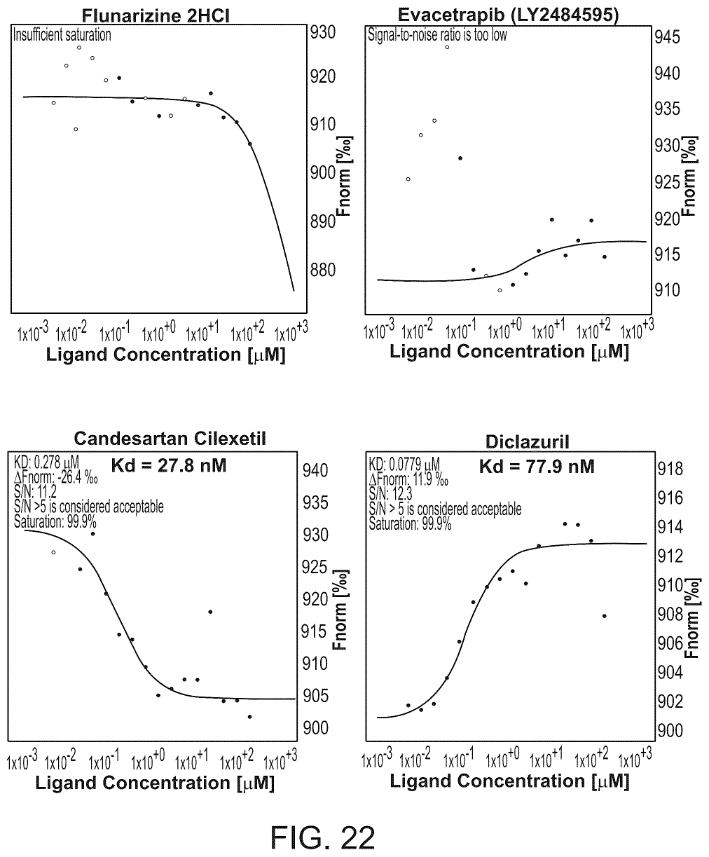

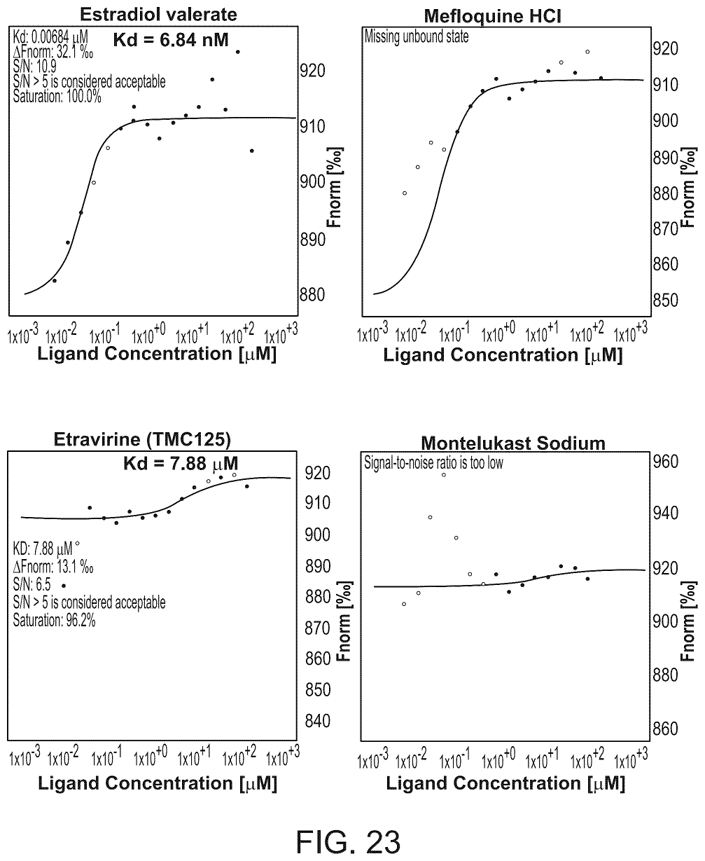

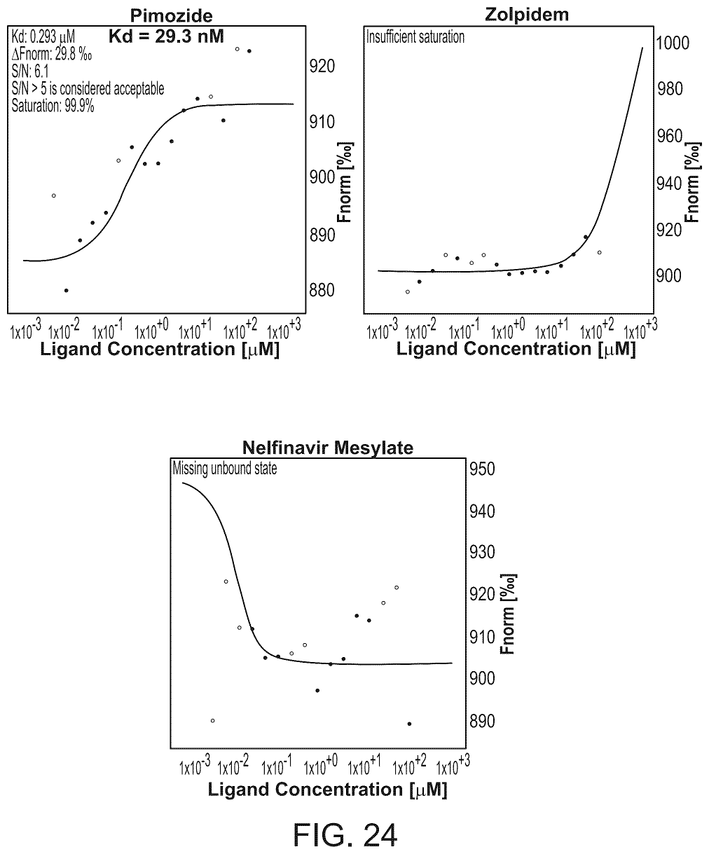

FIG. 19-24 are graphs of representative dose-response curves for select hits. Dissociation constants (Kd) calculated from curve fitting are displayed on the curve plots. Ivacaftor, nilotinib, zafirlukast, candesartan cilexetil, diclazuril, estradiol valerate, etravirine, and pimozide showed a response curve with signal to noise ratio greater than 5.

### DISCUSSION

The screen of FDA-approved small molecule drugs showed the discovery and validation of small molecule binders capable of binding to CA-IV for the purpose of delivering therapeutic agents across the BBB. These binders provide an avenue for the treatment of neurological diseases where drug delivery across the BBB remains a significant challenge.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A conjugate comprising:
a carbonic anhydrase IV (CA-IV) binder; and
a therapeutic cargo conjugated to the binder;
wherein the binder is a small molecule selected from the group consisting of foretinib, tanespimycin, nilotinib, tozasertib, ivacaftor, linsitinib, epirubicin HCl, zosuquidar 3HCl, ziprasidone HCl, acetylcysteine, zafirlukast, elvitegravir, meclizine 2HCl, candesartan cilexetil, diclazuril, flunarizine 2HCl, evacetrapib, estradiol valerate, etravirine, montelukast sodium, nelfinavir mesylate, mefloquine HCl, pimozide, zolpidem, and hydroxyprogesterone caproate, or a derivative thereof.

2. The conjugate of claim 1, wherein the therapeutic cargo is conjugated to the binder via a linker.

3. The conjugate of claim 1, wherein the therapeutic cargo is covalently conjugated to the binder.

4. The conjugate of claim 1, wherein the therapeutic cargo is a biological molecule.

5. The conjugate of claim 4, wherein the biological molecule is selected from the group consisting of a nucleic acid, a protein, a peptide, an antibody, a nanobody, a lipid, a polysaccharide, and a combination thereof.

6. The conjugate of claim 1, wherein the therapeutic cargo is a non-biological molecule.

7. The conjugate of claim 1, wherein the conjugate is characterized by delivery of the therapeutic cargo across the blood brain barrier (BBB).

8. The conjugate claim 7, wherein the therapeutic cargo is a therapeutic cargo for the treatment of a disorder affecting the central nervous system.

9. The conjugate claim 8, wherein the CA-IV binder binds to the human CA-IV (CA4).

10. The conjugate of claim 9, wherein when provided to a cell expressing CA-IV as a surface protein, binding of the binder to the CA-IV protein mediates transcytosis of the therapeutic cargo across the BBB.

11. A method of delivering a therapeutic cargo across the BBB of a subject, the method comprising:
providing to a subject a conjugate comprising:
a carbonic anhydrase IV (CA-IV) binder; and
a therapeutic cargo conjugated to the binder;
wherein the binder is discovered by a screening method comprising the steps of:
adding to a buffer CA-IV and a plurality of small molecules; and
measuring binding of the plurality of small molecules to CA-IV.

12. The method of claim 11, wherein the adding step comprises adding the CA-IV and plurality of small molecules to a plurality of wells.

13. The method of claim 11, wherein the conjugate further comprises a linker.

14. The method of claim 11, wherein the plurality of small molecules are small molecule drugs that have not previously shown binding to CA-IV.

15. The method of claim 11, wherein the binder is selected from the group consisting of foretinib, tanespimycin, nilotinib, tozasertib, ivacaftor, linsitinib, epirubicin HCl, zosuquidar 3HCl, ziprasidone HCl, acetylcysteine, zafirlukast, elvitegravir, meclizine 2HCl, candesartan cilexetil, diclazuril, flunarizine 2HCl, evacetrapib, estradiol valerate, etravirine, montelukast sodium, nelfinavir mesylate, mefloquine HCl, pimozide, zolpidem, and hydroxyprogesterone caproate, or a derivative thereof.

16. The conjugate of claim 11, wherein the therapeutic cargo is conjugated to the binder via a linker.

17. The conjugate of claim 11, wherein the therapeutic cargo is covalently conjugated to the binder.

18. The conjugate of claim 11, wherein the therapeutic cargo is a biological molecule.

19. The conjugate of claim 18, wherein the biological molecule is selected from the group consisting of a nucleic acid, a protein, a peptide, an antibody, a nanobody, a lipid, a polysaccharide, and a combination thereof.

20. The conjugate of claim 11, wherein the therapeutic cargo is a non-biological molecule.

21. (canceled)

22. (canceled)

23. (canceled)

24. (canceled)
